(19) United States
(12) Patent Application Publication
(10) Pub. No.: US 2021/0308001 A1
(43) Pub. Date: Oct. 7, 2021

(54) Shockwave and Pressure Waves for Treatment of Virus or Bacteria-Induced Effects in Human or Animal Lungs

(71) Applicant: SANUWAVE, INC., Suwanee, GA (US)

(72) Inventor: Iulian Cioanta, Milton, GA (US)

(73) Assignee: SANUWAVE, INC., Suwanee, GA (US)

(21) Appl. No.: 17/221,562

(22) Filed: Apr. 2, 2021

Related U.S. Application Data
(60) Provisional application No. 63/004,428, filed on Apr. 2, 2020.

(51) Int. Cl. A61H 23/00; A61B 17/22

(52) U.S. Cl. CPC A61H 23/008; A61B 2017/00809; A61B 17/22012

(57) ABSTRACT

Shockwaves or modulated pressure waves are applied to targeted lung tissue of humans or animals, including lung tissue having a viral or bacterial infection or a chronic lung condition, to destroy or render innocuous different pathogens and treat the underlying chronic or acute medical conditions.

### CROSS-REFERENCE TO RELATED APPLICATIONS

This application claims the benefit of priority of U.S. Provisional Application No. 63/004,428, filed Apr. 2, 2020, which is incorporated herein by reference in its entirety.

### BACKGROUND OF THE INVENTION

The present invention generally shows methods and devices or medical systems that produce and use focused, unfocused, planar, pseudo-planar or radial extracorporeal or intracorporeal specially modulated acoustic pressure focused shockwaves or specifically modulated pressure waves (that can be planar, pseudo-planar, radial, or unfocused waves) for treating acute or chronic lung conditions (for both humans and animals) by using a personalized approach that could positively influence the successful outcome of the treatment. Practically, the dosage of acoustic pressure shockwaves or pressure waves used for treatment is adjusted accordingly via algorithms that employs specific factors that take into account patient's condition and other parameters related to the state of damaged tissue, comorbidities and life style.

According to the published scientific literature for more than four decades, the extracorporeal acoustic pressure shockwaves and some specific pressure waves demonstrated positive results in treating different humans and animals soft or hard tissues, for healing diverse chronic wounds (diabetic foot ulcers—DFUs, arterial ulcers, venous ulcers, pressure sores, to name a few), and acute wounds, in inflammation elimination for different tissues as fascia or articular capsules and ligaments/tendons associated with the major synovial joints, in reduction and elimination of pain, in re-vascularization and prevention of ischemia, and in accelerated healing of bone fractures/non-unions. Intracorporeal shockwaves or specific pressure waves are used to treat arterial plaques and in general cardiovascular and endovascular problems. Furthermore, evidence was also produced about positive effects of shockwaves and pressure waves on killing planktonic bacteria and destroying bacterial, fungal, or other micro-organisms biofilms, with significant implication on treating infections for both humans and animals. As viruses can generate human or animal infections and, in many ways, they act the same as bacteria, although their dimensional scale and structure is different, most probably the bactericide effects of the acoustic pressure shockwaves or pressure waves can be translated to viruses, which can result in their eradication or ameliorated symptoms. Even more, based on the scientific literature, the shockwaves or pressure waves can modulate the immune response, intermediated by leukocytes, cytokines and matrix metalloproteinases (MMPs) activity, to not go in overdrive, which has significant consequences on treating any kind of external or internal infections of the human or animal body.

For any of the above-mentioned afflictions, in general the treatment regimens that employ acoustic pressure shockwaves and some specifically modulated pressure waves are fixed/predetermined (number of pulses, frequency, input energy setting, treatment duration, and total number of treatments) for each specific condition. The penetration and the power of shockwaves/pressure waves are dictated by the shape of the reflector used to reflect the shockwaves/pressure waves from the point of origin and towards the targeted treatment region/area.

In general, the acoustic pressure shockwaves and some specifically modulated pressure waves produced by the proposed embodiments will have a compressive phase (produces compressive pressures) and a tensile phase (negative pressures that produce cavitation bubbles, which collapse with high-speed jets) during one cycle of the acoustic pressure shockwaves or pressure waves. These two synergetic effects, work in tandem, by acting at macro (compressive phase) and micro level (cavitation jets of the tensile phase), which is enhancing the effects of the acoustic pressure shockwaves/pressure waves in the living tissue.

The high mechanical tension and pressures found at the front of the acoustic pressure shockwave distinguishes the acoustic pressure shockwaves from other kinds of sound waves, such as ultrasonic waves or other types of pressure waves. The acoustic pressure shockwaves generated for medical purposes consist of a dominant compressive pressure pulse, which climbs steeply up to maximum one hundred Mega-Pascals (MPa; 1 MPa=10 bar) within tens or hundreds of nanoseconds and then falls back to zero within a few microseconds. The final portion of the acoustic pressure shockwave pressure profile is characterized by low negative pressures (tensile region of the acoustic pressure shockwave), with potential to generate cavitation in body fluids. The bubble diameter grows as the energy is delivered to the bubble. This energy is released from the bubble during its collapse (implosion) in the form of high-speed pressure micro jets and localized/transient high temperature. The micro jets and elevated temperature are present within focal microscopic tissue volumes and are transient in nature.

Acoustic pressure shockwaves behave similarly to other sound waves, with the main difference that the acoustic pressure shockwaves possess more energy and allows the full development of the cavitational effect. An acoustic pressure shockwave can travel large distances easily (based on the amount of energy put in them at the point of origination) in a uni-directional fashion, as long as the acoustic impedance of the medium remains the same. The same acoustic impedance principle is valid for pressure waves, with the caveat that their energy is smaller when compared with shockwaves with consequences on their relatively limited traveling distance/penetration inside the tissue. Also, some pressure waves are not capable to produce viable cavitation bubbles that collapse with micro-jets, but rather oscillating small cavitational bubbles. At the point where the acoustic impedance changes, energy is released and the acoustic pressure shockwave or pressure waves are reflected or transmitted with attenuation. Thus, the greater the change in acoustic impedance in between different substances, the greater the release of energy is generated. Based on this principle of energy deposition inside the leaving tissue, when the acoustic impedance changes, the acoustic pressure shockwaves are used in medical field to break kidney or gallbladder stones, to stimulate tissue healing, and regenerate bones, muscle, skin, tendons, or any semi-soft and soft tissue from human or animal body.

In conclusion, the difference in between shockwaves and pressure waves is the amount of energy they deposit inside the treatment area and sometimes penetration inside the human body. Shockwaves are more powerful in general and deposit more energy in the targeted tissue due to their higher compressive pressures produced in the compressive phase and larger negative pressure from the tensile phase, which can produce more powerful cavitation in any bodily fluid. On their turn, the pressure waves are having a pressure signal flatter and more sinusoidal in shape, and due to their lower positive pressures and smaller values for negative pressures, they will put less energy inside the treatment zone. Sometimes this lower energy can be beneficial for specific applications where the targeted tissue is more delicate in nature, as for example in lungs or brain.

For acoustic pressure shockwaves (both extracorporeal or intracorporeal) to be effective in the clinical applications, they are sent in a predetermined direction (uni-directional) towards the point or region at which treatment is to be provided and for that they must be focused or concentrated (semi-focused) or unfocused or radially oriented. In general, in the treated region there are two basic effects, with the first being characterized as direct generation of mechanical forces (primary effect from the positive, compressive high pressure rise), and the second being the indirect generation of mechanical forces (high velocity pressure micro jets) produced by cavitation from the negative, tensile pressure region.

In lithotripsy, cavitation is believed to be the primary cause of stone disintegration. In orthopedics, the acoustic pressure shockwaves or specific pressure waves (planar, radial, or unfocused waves) have been shown to positively affect bone and soft tissue, beginning a regeneration process, due to synergy between the high compressive pressures applied to the tissue (macro effect) and the collapse of the cavitation bubbles with high-speed micro jets from the tensile phase (micro effect). For soft tissue treatment (as in pressure sores, chronic arterial and venous ulcers, diabetic foot ulcers, burns, all sorts of skin conditions, etc.), the acoustic pressure shockwaves or specifically modulated pressure waves (planar, pseudo-planar, radial, or unfocused waves) are highly controlled to generate an energy output that will not produce any tissue injury. This is accomplished based on reflector geometry and material, input energy setting (input energy level of the acoustic pressure shockwaves or pressure waves), number of acoustic pressure shockwaves or pressure waves (planar, pseudo-planar, radial, or unfocused waves), and their frequency per second that dictates the total acoustic energy delivered in one treatment session. Reflector geometry directly controls the delivery of the shockwaves or pressure waves (focused or unfocused) into the targeted treatment region and shapes their spatial distribution in the targeted treatment region.

The input energy settings (energy input into acoustic pressure shockwaves or specifically modulated pressure waves (planar, pseudo-planar, radial, or unfocused waves)) directly affect the pressure output into the targeted treatment region/zone and together with the number of acoustic pressure shockwaves/pressure waves and their frequency per second, determine the total amount of energy deposited inside the tissue from the targeted treatment region. The peak positive compressive pressures of the extracorporeal or intracorporeal acoustic pressure shockwaves or specifically modulated pressure waves (planar, pseudo-planar, radial, or unfocused waves) are concentrated to a specifically localized region causing a macro tissue disruption, movement or stretching of the tissue in the treatment region at amplitudes sufficient to disturb the tissue/cells but not cause damage, which initiates the cellular signaling for modulating inflammation and immune system response, influx of growth factors and other proteins. At the energy levels and dosages used in tissue repair and regeneration, cavitation occurring in the negative pressure phase is also triggering cellular signaling without any damage. Pyrolysis produced by cavitation may be responsible for the observed Reactive Oxygen Species (ROS) expression as evidenced by up-regulation of endothelial constitutive nitric oxide synthase (eNOS). The negative pressure of the tensile phase may also release oxygen bound to plasma and hemoglobin and thus becoming a source of the immediate increased oxygenation, which attracts macrophages to the treatment site and begins the signaling for chemokines and cytokines of the immune system, as observed in clinical studies.

The tissue (macro level) and cellular (micro level) disruptions are hypothesized to be the source of cellular expression seen in the laboratory and clinical work, when tissue is treated with extracorporeal or intracorporeal acoustic pressure shockwaves or specifically modulated pressure waves (planar, pseudo-planar, radial, or unfocused waves) that provides the necessary triggering to start or re-start the body's natural healing process. The initial response to the acoustic pressure shockwaves application promotes microcirculatory improvement. Continued effects after treatment include modulation of inflammatory/immune system and in existing blood circulation perfusion responses, followed by an increase in capillary perfusion and vessel permeability, cellular signaling that initiates angiogenic growth factors and proteins upregulation, and calls in the proangiogenic growth factors (as vessel endothelial growth factor—VEGF), leading to new small blood vessels formation that translates in increased vascularization and ultimately tissue regeneration and healing. As a result, a certain medical conditions condition can be resolved by jumpstarting the normal body's own healing response.

The increase in blood perfusion is important, as it is by definition a decrease in the ischemia (lack of blood flow and oxygenation) that is often associated with impaired tissue conditions healing. As the cells of the microcirculatory system or respiratory system are disrupted by the acoustic pressure shockwaves or specifically modulated pressure waves (planar, pseudo-planar, radial, or unfocused waves), there is an immediate change in local blood flow to the treatment targeted area/region, due to relaxation of local arterioles and increase in their diameter. This effect, in combination with unaltered blood flow, results in better perfusion and oxygenation of the targeted tissue, with implication in oxygenation and healing. The vessel permeability index (VPI) simultaneously increases directly after acoustic pressure shockwave or specific pressure waves treatment. This is a measure of the plasma or fluid content of the blood “leaking” from the vessel walls, and more fluid exchange increases the exchange of nutrients and gases between blood vessels, alveoli (lungs), and tissue cells in the treatment area. Thus, the extracorporeal or intracorporeal acoustic pressure shockwaves or specifically modulated pressure waves (planar, pseudo-planar, radial, or unfocused waves) show that more leukocytes (white blood cells) beginning to roll and stick to the blood vessel walls, finally transmigrating through the vessel wall and into the treatment region. Increasing leukocyte activation assists in the inflammatory phase of healing and an inflammatory modulation response is apparent after acoustic pressure shockwave or specifically modulated pressure waves (planar, pseudo-planar, radial, or unfocused waves) treatment. Down-regulation of inducible nitric oxide synthase (iNOS), which is an inflammatory marker, has been demonstrated along with an initial up-regulation of pro-inflammatory chemokines that come back to normal rapidly afterwards, which shortens the inflammatory phase duration. The treatment is shown to blunt poly morphonuclear neutrophil and macrophage infiltration into the targeted tissue, which further reduces the excessive inflammatory state. In addition, there is a suppression of pro-inflammatory cytokines (IL-1β, IL-6, and TNFα) and downregulation of matrix metalloproteinases (MMPs) and TIMP-1 metallopeptidase inhibitor 1 in response to acoustic pressure shockwave or specifically modulated pressure waves (planar, pseudo-planar, radial, or unfocused waves) treatment, which is important in modulation of the inflammation and immune system response, since the significant increases of such factors is usually associated with overexpression of immune response that can produce impaired healing, sepsis, or even death.

In conclusion, these factors effectively allow a distressed tissue to move through the inflammatory phase quickly and switch to healing and the proliferation (cell growth) phase. The shortened inflammatory phase, noticed after extracorporeal shockwaves (ESWs) or intracorporeal shockwaves (ISWs) or specifically modulated pressure waves (planar, pseudo-planar, radial, or unfocused waves) treatment, may be beneficial to long-term healing and prevention of scar tissue formation in the infected/diseased tissues, natural conduits, glands or organs. The reduction of scarring has significant implication in preserving the functional integrity of the respective tissue, natural conduit, gland or organ after the healing period.

### SUMMARY OF THE INVENTION

Numerous published scientific papers demonstrate that the mechanisms of action of focused, unfocused, planar, pseudo-planar or radial extracorporeal shockwaves (ESWs) or intracorporeal shockwaves (ISWs) or specifically modulated pressure waves (planar, pseudo-planar, radial, or unfocused waves) involve the use of oxygen radicals, attenuation of leukocyte infiltration, global suppression of pro-inflammatory cytokines and matrix metalloproteinases (MMPs), which ultimately modulates inflammation in injured tissues. That is followed by the decrease in tissue apoptosis and recruitment of different growth factors, which can heal or regenerate faster acute and chronic afflictions of tissues, natural conduits, glands or organs from both humans and animals.

Also, the shockwaves (ESWs or ISWs) and specifically modulated pressure waves (planar, pseudo-planar, radial, or unfocused waves) produce a kinetic effect on tissues and organs due to the forces generated by the compressive positive pressures of their compressive phase and due to the forces associated with micro-jets produced during cavitation bubbles collapse from their tensile phase, as mentioned elsewhere in this disclosure. That can have a benefic influence is dislodging different pathogens as viruses, bacteria, fungus, micro-organisms, mucus, and any type of biofilms formed inside the tissues, natural conduits, glands and organs of a human or animal body.

In the last decades, there were new types of viruses that produced severe infections in humans or seasonal re-occurrence of certain diseases as flu or influenzae. In this realm, the mouth, eyes, nasal cavity, throat, lungs, stomach, intestines are susceptible areas and organs to such infections, since there is a conduit or conduits linked to them that gives a direct pathways access for the external pathogens to penetrate inside the human or animal body.

Within the lungs, the bronchial tubes branch many times into thousands of smaller, thinner tubes called bronchioles (see FIG. 9). These tubes end in bunches of tiny round air sacs called alveoli, as presented in FIG. 8. The alveoli are where oxygen and carbon dioxide are exchanged. The tissues of the respiratory tract are thin and delicate, and become thinnest at the surfaces of the alveoli, where gaseous exchange occurs. Small blood vessels called capillaries run along the walls of the air sacs. When air reaches the air sacs, oxygen passes through the air sac walls into the blood in the capillaries. At the same time, a waste product, called carbon dioxide (CO₂) gas, moves from the capillaries into the air sacs. This process, called gas exchange, brings in oxygen for the body to use for vital functions and removes the CO₂. The airways and air sacs are elastic or stretchy. When a living organism breathe in, each air sac fills up with air, like a small balloon and when breathe out, the air sacs deflate and the air goes out. The body has a number of mechanisms, which protect these tissues and ensure that debris and bacteria do not reach them. Tiny hairs called cilia trap large pieces of debris and waft them out of the airways. For that the reflexes of sneezing and coughing help to expel particles from the respiratory system. In the same time, the production of mucus keeps the tissues moist and helps to trap small particles of foreign matter such as dust particles, bacteria, viruses and other inhaled debris. Mucus production in the airways is normal and it contains glycoproteins (or mucins), natural antibiotics, which help to destroy bacteria, as well as proteins derived from plasma, and products of cell death such as DNA. Without it, airways become dry and malfunction. In the cases of pathogens infections and some chronic diseases of the lung, the mucus is produced in excess and changes in nature. This results in the urge to cough and expectorate this mucus as sputum. Sputum production is associated with many lung diseases processes and it may become infected, stained with blood or contain abnormal cells.

For the respiratory system, the humanity witnesses severe viral infections (originated from viruses) as the Severe Acute Respiratory Syndrome (SARS-CoV) in 2003, porcine flu in 2009, Middle East Respiratory Syndrome (MERS) in 2015, and lately the Corona Virus Disease (COVID-19) in 2019/2020/2021, which points out the human vulnerability against virulent respiratory viruses. The existing evidence on coronaviruses suggests that they may follow the pattern seen in influenza. These viruses have spike proteins or grabbers that hook onto host cells cleavage site that allows the virus to open and enter those cells. When harmful pathogens invade and start to reproduce, the host immune system recognizes them by their shapes. The pathogens have antigens, which are special proteins that trigger an attack from the body's immune system. These antibodies attach to antigens on the pathogens and prevent pathogens from invading other cells. At their turn, the antibodies signal white blood cells, which kill and remove the pathogens. In general, a specific shape of antibody is needed. The human body has billions of white blood cells, each making its own special-shaped antibody. Only a few antibody shapes will be effective against on specific pathogen. It can take several days for the immune system to produce enough properly shaped antibodies to kill the invading pathogens. During that time, a fast-acting pathogen, which can replicate billions of copies of itself, is a critical health threat. In severe cases of COVID-19, patients experience pneumonia, which means their lungs begin to fill with pockets of pus or fluid. This leads to intense shortness of breath and painful coughing. In general, after such severe viral infections, the lung is damaged and not enough oxygen is supplied to the rest of the body, which can trigger the respiratory failure that could lead to organ failure and death.

These viral infections can produce fever, respiratory symptoms as dry cough and shortness of breath, myalgia or fatigue, weight loss, cell debris-filling bronchiolar lumen, alveolar collapse with hemorrhage, and radiological “ground-glass” lung opacities. It was found that the higher initial load of virus in the nasopharynx, the cavity in the deep part of the throat, was correlated with a more severe respiratory illness. Nearly all the SARS patients who came in initially with a low or undetectable level of virus in the nasopharynx were found up to be still alive at two-month follow-up, which is in sharp contrast compared to those with the highest level who had a twenty- to forty-per-cent mortality rate. In a paper published online by The Lancet Infectious Diseases in March 2020, researchers at the University of Hong Kong and Nanchang University reported that viral loads in nasopharyngeal swabs from a group of patients with severe COVID-19 were sixty times higher, on average, than the loads among patients with a mild form of the disease. Logically, the larger amount of virus should trigger more severe disease by prompting a brisker inflammatory response, which in some cases can generate a “viral cytokine storm” with detrimental effects. Cytokines are a group of proteins secreted by cells of the immune system that act as chemical messengers. Cytokines released from one cell affect the actions of other cells by binding to receptors on their surface. A cytokine storm is an overproduction of immune cells and their activating compounds, which in a flu infection is often associated with a surge of activated immune cells into the lungs. Cytokine storms are also suspected to be the main cause of death in the 1918 “Spanish Flu” pandemic. Deaths were weighted more heavily towards people with healthy immune systems, due to their ability to produce stronger immune responses, with dramatic increases in cytokine levels. Despite the use of vaccines and antiviral therapies such as oseltamivir, severe influenza kills many people each year. Flu infection often leads to a vigorous immune response and body-wide inflammation, leading to the hallmark symptoms of high fever, cough, headache, muscle and joint pain, and severe fatigue. However, when the inflammation becomes excessive, driven by the overproduction of inflammatory mediators called cytokines, this “cytokine storm” can rapidly kill cells, causing severe tissue damage while precipitating organ dysfunction and failure, particularly of the lungs, kidneys, and circulatory system. Immunomodulatory therapy has been proposed as a possible way to improve the outcome, with or without antiviral agents. It seems that the same thing is happening for the COVID-19 patients who are severely affected by the disease and it is life-threatening for these patients.

Anti-inflammatory and immunosuppressive drugs have not been successful in treating cytokine storm and improving survival for the COVID-19 patients. Nonsteroidal anti-inflammatory drugs (NSAIDS), such as ibuprofen, are used commonly to treat mild to moderate inflammation, but have not demonstrated the ability to control cytokine storm. Similarly, corticosteroids have also had mixed results, and their use for treating severe influenza is not recommended due to an increased risk of hospital-acquired infections and death. Other experimental approaches, such as the use of statins, zinc-based products, etc. have shown some success when used with other agents to alter the body's immune response. However, due to all benefic effects that extracorporeal shockwaves (ESWs) or intracorporeal shockwaves (ISWs) or specifically modulated pressure waves (planar, pseudo-planar, radial, or unfocused waves) showed in the treatment of bacterial infections, should easily translate also in the treatment of viral infections, as COVID-19, flu, and other viruses.

Bronchitis is an inflammation of the bronchial lining, as seen in FIG. 9. It is commonly related to cigarette smoking but is also triggered by environmental irritants such as chemical vapors, exhaust fumes, or pesticides. In response to the inflammation, excess mucus is produced. This can block the small airways and reduce respiratory efficiency, in chronic airways obstruction. Over-production of mucus leads to frequent coughing, which further irritates the tissues and causes even more mucus production. The mucus dislodging and movement/elimination from the airways be accomplished by treatments with extracorporeal shockwaves (ESWs) or intracorporeal shockwaves (ISWs) or specifically modulated pressure waves (planar, pseudo-planar, radial, or unfocused waves) or in combination with other therapies.

Pneumonia is another disease of the lungs where the thin layer of alveolar cells is damaged by a virus. The body reacts by sending immune cells to the lung to fight it off and that results in the alveolar linings becoming thicker than normal, as seen in FIG. 8. As they thicken more and more, they essentially choke off the little air pocket of the alveoli, which has big influence on oxygenation in general. Restricting oxygen to the bloodstream deprives other major organs of oxygen including the liver, kidney, and brain. Pneumonia is characterized by shortness of breath combined with a cough and affects tiny air sacs in the lungs. The hyperactive immune response and elimination of the viral pathogen can be accomplished by treatments with extracorporeal shockwaves (ESWs) or intracorporeal shockwaves (ISWs) or specifically modulated pressure waves (planar, pseudo-planar, radial, or unfocused waves) or in combination with other therapies.

Chronic obstructive pulmonary disease (COPD) is a lung disease characterized by chronic obstruction of lung airflow that interferes with normal breathing, as presented in FIG. 9. The more familiar terms ‘chronic bronchitis’ and ‘emphysema’ are no longer used, but are now included within the COPD diagnosis. Chronic bronchitis is inflammation of the lining of the bronchial tubes, which carry air to and from the air sacs (alveoli) of the lungs. It is characterized by daily cough and mucus (sputum) production. Emphysema is a condition in which the alveoli at the end of the smallest air passages (bronchioles) of the lungs are destroyed as a result of damaging exposure to cigarette smoke and other irritating gases and particulate matter. The delirious effects of COPD can be ameliorated or eliminated by treatments with extracorporeal shockwaves (ESWs) or intracorporeal shockwaves (ISWs) or specifically modulated pressure waves (planar, pseudo-planar, radial, or unfocused waves) or in combination with other therapies.

Another pulmonary disease that can be treated with shockwaves (both ESWs or ISWs) and specifically modulated pressure waves (planar, pseudo-planar, radial, or unfocused waves) is the idiopathic pulmonary fibrosis (IPF). This disease is a progressive interstitial lung disease that develops as a result of overexuberant remodeling following pulmonary epithelial damage, and which is characterized by chronic inflammation, alveolar epithelial hyperplasia, and deposition of extracellular matrix leading to development of a permanent “scar”. It was demonstrated that in this disease, also the macrophages have a role in generating the pathological process. Lung fibrosis mediated by the recruitment of macrophages to the lung, which in the end can damaged the lungs. Based on the known reduction in macrophages activity, modulation of inflammation and hyperplasia, reduction or elimination of fibrotic tissue and scars, which are usually generated in the wound healing by shockwaves or specifically modulated pressure waves (planar, pseudo-planar, radial, or unfocused waves), it is clear that benefic effects preferably are seen into idiopathic pulmonary fibrosis (IPF), when such technology is used for treatment.

Interesting to note that extracorporeal shockwaves (ESWs) or intracorporeal shockwaves (ISWs) or specifically modulated pressure waves (planar, pseudo-planar, radial, or unfocused waves) besides modulating the inflammation and thus producing a reduction in the scar tissue in general, at certain dosages can also remove old scar tissue and replace it with normal tissue, which has implication post-infection. By eliminating the scar tissue present in the lungs due to previous infections, allows a better capacity for the lungs and overall an increase in lung oxygen output to the body and organs, which is essential for their good health and functionality.

In another severe pulmonary known as cystic fibrosis, the inflammatory damage is a severe problem, with 85% of deaths as a result of persistent inflammation triggered by recurrent rounds of bacterial infection, clearance, inflammation, and remodeling. Cystic fibrosis is a lethal disease that is inherited and affects Caucasians of North European descent. A defective gene located on chromosome 7 means a protein called a cystic fibrosis transmembrane regulator, responsible for the active transport of chloride ions within cells, does not function normally. This protein is abundant in cells that produce watery secretions such as mucus. The abnormality means that secretory cells cannot transport salts and water efficiently, and secretions become thick. The sticky mucus adheres to the airways and cannot be transported properly and mucus plugs block the smaller airways. Breathing becomes difficult and problems with transporting mucus may lead to bacterial colonization. Cystic fibrosis patients that initiate infections, usually result in overwhelming inflammation, cell death, and sepsis. This hyperreaction by pulmonary macrophages, which triggers overwhelming cytokine production and the inflammatory cytokine storm that can induce pathology and severe lung damage. The delirious effects of cystic fibrosis can be ameliorated or eliminated by treatments with extracorporeal shockwaves (ESWs) or intracorporeal shockwaves (ISWs) or specifically modulated pressure waves (planar, pseudo-planar, radial, or unfocused waves) or in combination with other therapies.

In general, the clearance of the infecting pathogens comes at a heavy price as the inflammatory mediators can themselves lead to bystander damage of lung tissue. Attenuating the post-pathogen cytokine storm observed in severe cases of COVID-19, cystic fibrosis, and chronic pulmonary obstructive disease, by using a multitude of immunomodulatory mechanisms to treat active infection and post-viral sequelae, it is an important factor in the treatment of such infections. Based on the mechanism of action for extracorporeal shockwaves (ESWs) or intracorporeal shockwaves (ISWs) or specifically modulated pressure waves (planar, pseudo-planar, radial, or unfocused waves) it seems that they can play a role in modulating inflammation, immune system response to tone down the cytokine storm surge.

In treating such afflictions produced by new pathogens as COVID-19, the immediate treatment approach is the use of local or systemic medication, or drugs, or antibiotics, or homeopathic agents, which are targeting the specific invading organism. This medication approach to the treatment is hindered by the location in the tissue (fibrous or scar tissue) where bacteria, viruses, funguses and other harmful micro-organisms are active, making the medication, or drugs, or antibiotics, or homeopathic agents ineffective due to inflammation and poor oxygenation of the respective tissue, which prevents medication, or drugs, or antibiotics, or homeopathic agents to reach the infected tissue. The medication approach can also be hindered by the resistance for a specific medication, or drugs, or antibiotics, or homeopathic agents and by the tolerance of the patient to the respective treatment regimen. Also, in the case of medication, although in general potent and with immediate impact, the biochemical resistance of bacteria and viruses to antimicrobial agents may occur by mutation, natural selection, transformation, transduction or conjugation, which produces in some cases antibiotic resistance. Bacteria initially sensitive to an antimicrobial agent may become resistant, and another antimicrobial agent must then be used. The global concerns for developing antimicrobial drug resistance and the need to develop more prudent and judicious use of drugs have caused the necessity of finding new approaches to treat infections that do not display these disadvantages. This is a big advance of the extracorporeal shockwaves (ESWs) or intracorporeal shockwaves (ISWs) or specifically modulated pressure waves (planar, pseudo-planar, radial, or unfocused waves), since their pure mechanical action prevents the development of any possible adaptation or resistance by a certain type of pathogen.

It is clear that the humanity needs to increase the options available to treat such infections, besides specific medication, vaccination for prevention, and mechanical devices to take care of different severe symptoms related to such infections (ventilators, intubation, catheterization, perfusion, etc.).

### BRIEF DESCRIPTION OF THE DRAWINGS

FIG. 1 is a schematic representation of features and characteristics for focused pressure shockwaves that are used in embodiments of the present invention.

FIG. 2 is a schematic representation of planar/pseudo-planar pressure waves propagation and pressure signal in a treatment zone, as used in embodiments of the present invention.

FIG. 3 is a schematic representation of a comparison between focused pressure shockwaves and radial pressure waves pressure profile in a treatment zone, as used in embodiments of the present invention.

FIG. 4 is a schematic representation of a comparison of power and penetration for focused pressure shockwaves, planar pressure waves, and radial pressure waves, as used in embodiments of the present invention.

FIG. 5 is a schematic representation of pressure profiles comparing focused and unfocused shockwaves, as used in embodiments of the present invention.

FIG. 6 is a schematic representation comparing pressure energy output based on size of reflector aperture for producing focused pressure shockwaves, as used in embodiments of the present invention.

FIG. 7 is a prior art illustration of the destruction of bacterium integrity by focused pressure shockwaves and pressure waves created by planar, unfocused, and radial waves, as relates to embodiments of the present invention.

FIG. 8 is a prior art illustration of the normal and disease affected tissues and airways in the lungs, as relates to embodiments of the present invention.

FIG. 9 is a prior art illustration of chronic obstructive pulmonary disease (COPD) manifestation, as relates to embodiments of the present invention.

FIG. 10 is a schematic representation of shockwaves or pressure waves applied at an acute angle relative to the target tissue to create both normal and tangential forces at the intersection with the target, as used in embodiments of the present invention.

FIG. 11 is a schematic representation of an inclined applicator configured to deliver focused acoustic shockwaves for treating lung afflictions, according to one embodiment of the present invention.

FIG. 12 is a schematic lateral view representation of an adjustable handle that allows angular pivoting for the inter-rib spaces and vertical adjustment for variable penetration used for treating lung afflictions, according to one embodiment of the present invention.

FIG. 13 is a schematic top view representation of an adjustable handle of FIG. 12, according to one embodiment of the present invention.

FIG. 14 is a schematic representation of a spark-gap electrohydraulic applicator producing focused shockwaves for treating lung afflictions, according to one embodiment of the present invention.

FIG. 15 is a schematic representation of a laser electrohydraulic applicator producing focused shockwaves for treating lung afflictions, according to one embodiment of the present invention.

FIG. 16 is a schematic representation of a piezoelectric-crystals/piezo ceramics applicator producing focused shockwaves for treating lung afflictions, according to one embodiment of the present invention.

FIG. 17 is a schematic representation of piezoelectric fibers applicator producing focused shockwaves for treating lung afflictions, according to one embodiment of the present invention.

FIG. 18 is a schematic representation of an electromagnetic lens applicator producing focused shockwaves for treating lung afflictions, according to one embodiment of the present invention.

FIG. 19 is a schematic representation of an electromagnetic coil applicator producing focused shockwaves for treating lung afflictions, according to one embodiment of the present invention.

FIG. 20 is a schematic representation of a hydraulic applicator producing focused shockwaves for treating lung afflictions, according to one embodiment of the present invention.

FIG. 21 is a schematic representation of a pneumatic applicator producing radial pressure waves for treating lung afflictions, according to one embodiment of the present invention.

FIG. 22 is a schematic representation of a spark-gap electrohydraulic applicator producing focused shockwaves that has increased reflective area and it is used for lung afflictions, according to one embodiment of the present invention.

FIG. 23 is a schematic representation of a spark-gap electrohydraulic applicator producing radial pressure waves for treating lung afflictions, according to one embodiment of the present invention.

FIG. 24 is a schematic representation of a spark-gap electrohydraulic applicator producing multiple radial pressure waves that has multiple spark-gaps and it is used for treating lung afflictions, according to one embodiment of the present invention.

FIG. 25 is a schematic representation of a laser electrohydraulic applicator producing radial pressure waves that has multiple spark-gaps and it is used for treating lung afflictions, according to one embodiment of the present invention.

FIG. 26 is a schematic representation of a spark-gap electrohydraulic applicator for producing pseudo-planar pressure waves used to treat lung afflictions, according to one embodiment of the present invention.

FIG. 27 is a schematic representation of a piezoelectrical crystals applicator for producing planar pressure waves used to treat lung afflictions, according to one embodiment of the present invention.

FIG. 28 is a schematic representation of an applicator with segmented reflector producing pseudo-planar pressure waves that is used for the treatment of lung afflictions, according to one embodiment of the present invention.

FIG. 29 is a schematic representation of a spark-gap electrohydraulic applicator with segmented reflector producing pseudo-planar pressure waves for the treatment of lung afflictions, according to one embodiment of the present invention.

FIG. 30 is a schematic representation of a laser electrohydraulic applicator with segmented reflector producing pseudo-planar pressure waves for the treatment of lung afflictions, according to one embodiment of the present invention.

FIG. 31 is a schematic representation of a piezoelectric-crystals/piezo ceramics applicator with segmented reflector producing pseudo-planar pressure waves for the treatment of lung afflictions, according to one embodiment of the present invention.

FIG. 32 is a schematic representation of an electromagnetic coil applicator with segmented reflector producing pseudo-planar pressure waves for the treatment of lung afflictions, according to one embodiment of the present invention.

FIG. 33 is prior art illustrating the classic semi-ellipsoidal (half of an ellipsoid) reflector geometry and characteristics, as relates to embodiments of the present invention.

FIG. 34 is prior art illustrating the reversed semi-ellipsoidal reflector geometry and characteristics, as relates to embodiments of the present invention.

FIG. 35 is a schematic representation of a lateral view of a spark-gap electrohydraulic applicator with reversed reflector producing both focused shockwaves and radial pressure waves for the treatment of lung afflictions, according to one embodiment of the present invention.

FIG. 36 is a schematic representation of the top view of a spark-gap electrohydraulic applicator with reversed reflector as presented in FIG. 35, according to one embodiment of the present invention.

FIG. 37 is prior art illustrating a reflector with movable spark-gap that produces focused and defocused shockwaves resulting in a combination of focal volumes, as relates to embodiments of the present invention.

FIG. 38 is a schematic representation of a lateral view of a spark-gap electrohydraulic applicator with reversed reflector and movable spark-gap discharge producing focused and defocused shockwaves and radial pressure waves for the treatment of lung afflictions, according to one embodiment of the present invention.

FIG. 39 is prior art illustrating intracorporeal catheters having different constructions.

FIG. 40 is prior art illustrating an intracorporeal catheter producing shockwaves at its distal end, as it relates to embodiments of the present invention.

FIG. 41 is a schematic representation of an intracorporeal shockwave or pressure wave catheter for treating the tracheal trunk and removing mucus or sputum, according to one embodiment of the present invention.

FIG. 42 is prior art illustrating a pipe reflector with semi-ellipse cross section, as relates to embodiments of the present invention.

FIG. 43 is a schematic representation of an intracorporeal shockwave or pressure wave catheter with a pipe reflector for treating the tracheal trunk and removing mucus or sputum, according to one embodiment of the present invention.

### DETAILED DESCRIPTION OF THE INVENTION

For infections of the lungs an alternative treatment, including in certain embodiments in combination with other therapies (such as drug, ultrasound, mechanical, and stem cell therapies), is the use of focused, unfocused, planar, pseudo-planar or radial extracorporeal or intracorporeal acoustic pressure shockwaves or specifically modulated pressure waves (planar, pseudo-planar, radial, or unfocused waves), which are known to have effects demonstrated in vitro and in vivo against a large gamut of pathogens under both static and dynamic growth conditions. Based on the demonstrated shockwave and pressure wave mechanism of action produced by their compressive and tensile phases on bacterium and bacterial biofilms, the extracorporeal and possible intracorporeal acoustic pressure shockwaves or specifically modulated pressure waves (planar, pseudo-planar, radial, or unfocused waves) have similar action on viruses, funguses and other harmful micro-organisms. Furthermore, localized/transient thermal effects created during collapse of the cavitation bubbles can also kill bacteria and viruses. The acoustic pressure shockwaves extracorporeal and possible intracorporeal acoustic pressure shockwaves or specifically modulated pressure waves (planar, pseudo-planar, radial, or unfocused waves) can generate free radicals that have a potential destructive effect on bacteria, viruses or biofilms. Furthermore, fairly new research showed that the shockwaves and pressure waves are capable to open vesicles in lipid membranes for enveloped viruses, which might be another mechanism to destroy such viruses. Once the lipid viral envelop is destroyed the enveloped viruses are rendered harmless, since they are no longer able to recognize sites to identify and attach the host cells. For the non-enveloped viruses, the external coat or envelop called capsid is made of proteins. The capsid of the non-enveloped viruses is vulnerable to the mechanical forces and stresses produced by the shockwaves and pressure waves, or to the action of free radicals, and also to the transient sonoluminescence and heat produced during collapse of the cavitation bubbles generated in the tensile phase by the negative pressures. In conclusion there are multiple mechanism of action that can destroy the integrity of a virus and also to render a virus harmless, which overall reduce the viral load with benefic effects in treating the viral infections.

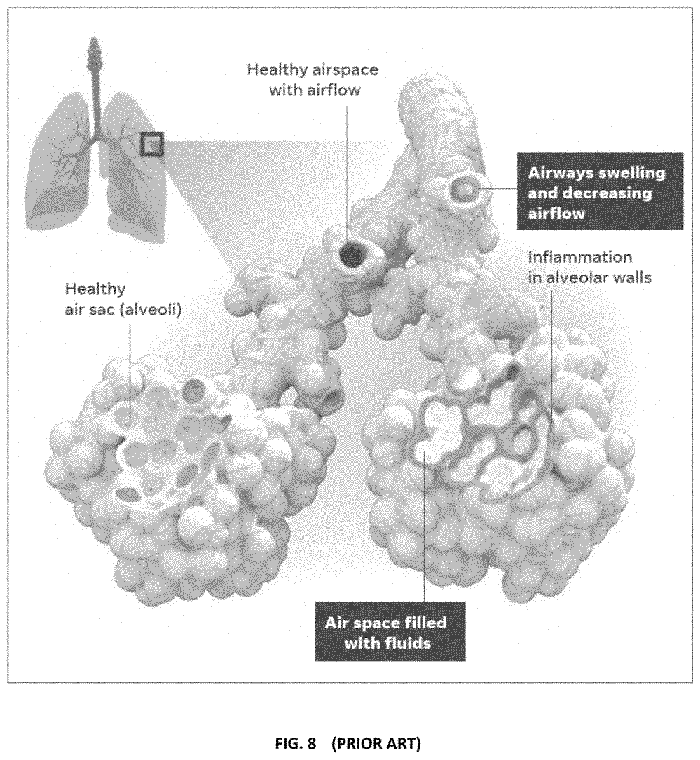

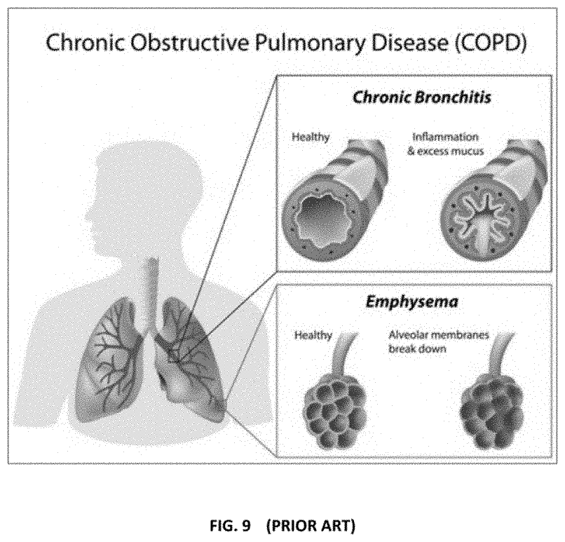

Also, the dislodging and destruction of possible biofilms and mucus (from the bronchial tubes branches and alveolar space, as seen in FIGS. 8 and 9) by the extracorporeal and possible intracorporeal acoustic pressure shockwaves or specifically modulated pressure waves (planar, pseudo-planar, radial, or unfocused waves) can also help with infection reduction and is allowing the natural immune system action. The fragmented pieces of biofilms, bacterium, viruses, or other micro-organisms can expose individual pathogens that were hiding from the immune system, which then can allow the immune system to recognize a pathogen and generate an appropriate natural reaction against it.

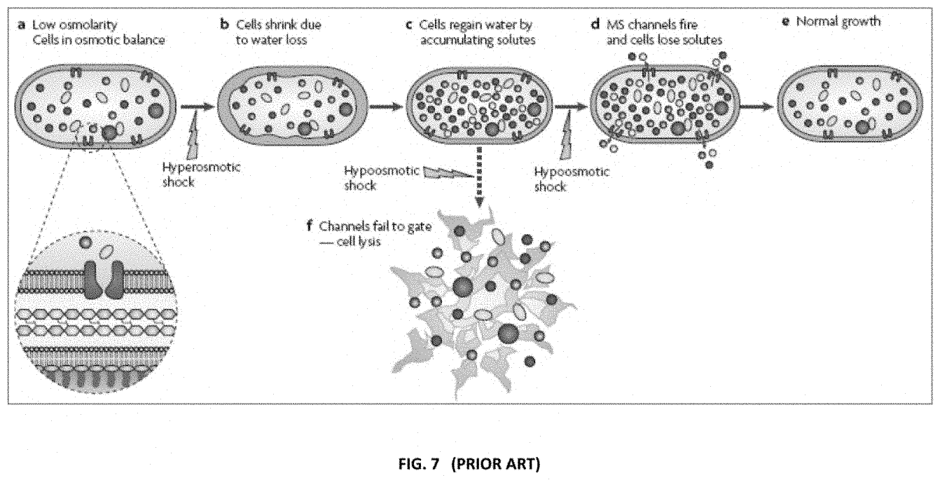

Thus, extracorporeal and intracorporeal acoustic pressure shockwaves or specifically modulated pressure waves (planar, pseudo-planar, radial, or unfocused waves) can destroy bacteria by affecting their membrane integrity. That is done via interference with bacterium mechano-transduction (transport of fluids across bacterium membrane), due to significant localized pressure variations produced by acoustic pressure shockwaves or specifically modulated pressure waves (planar, pseudo-planar, radial, or unfocused waves), as presented ion detail in FIG. 7. When the extracorporeal or intracorporeal acoustic pressure shockwaves or specifically modulated pressure waves (planar, pseudo-planar, radial, or unfocused waves) are transmitted in the region where the pathogen exists, it generates sudden variation in pressure from high positive pressures to significant negative pressures that open pores in the bacterium membrane to facilitate the transport of fluids across membrane to equilibrate the osmotic pressures. In the simplest terms, the bacterium membrane mechanosensitive channels (MS) or pores remain opened due to sudden and continuous pressure changes and the bacteria swells until the integrity of the membrane is compromised. According to the published scientific literature, the bacterial membrane mechanotransduction occurs at pressures of approximately 3 MPa or less, which is exactly in the realm of pressure generated by acoustic pressure shockwaves or specifically modulated pressure waves (planar, pseudo-planar, radial, or unfocused waves).

For viruses, their shell integrity can be compromised due to the penetrating forces of the micro jets produced by the collapse of cavitational bubbles formed in the tensile phase of the acoustic pressure shockwaves or specifically modulated pressure waves (planar, pseudo-planar, radial, or unfocused waves). Another factor that can act on viruses are the transient high temperatures produced during cavitational bubbles collapse, which can denature the virus and its DNA material.

The extracorporeal and possible intracorporeal extracorporeal or intracorporeal acoustic pressure shockwaves or specifically modulated pressure waves (planar, pseudo-planar, radial, or unfocused waves) produce a pure mechanical process that is involved in the destruction of individual bacterium or virus. This is why no biochemical process is involved, which can potentially produce viral mutations. Thus, there is no developed resistance of bacteria or viruses to acoustic pressure shockwaves or specifically modulated pressure waves (planar, pseudo-planar, radial, or unfocused waves), due to their mutation, natural selection, transformation, transduction or conjugation.

Furthermore, extracorporeal or intracorporeal acoustic pressure shockwaves or specifically modulated pressure waves (planar, pseudo-planar, radial, or unfocused waves) can be used in conjunction with antibiotics or other medications for treating infections, or existing medical devices for treating superficial, profound, local or systemic infections, without any detrimental interference. The fact that they can be applied extracorporeally or if needed intracorporeally and can penetrate at any depth inside the human and animal body, without creating sustained heat or other significant side effects, makes the extracorporeal or intracorporeal acoustic pressure shockwaves or specifically modulated pressure waves (planar, pseudo-planar, radial, or unfocused waves) very easy to administer, as a standalone treatment or as an additional/additive treatment to other existing modalities used to treat infections. Also, extracorporeal or intracorporeal acoustic pressure shockwaves or specifically modulated pressure waves (planar, pseudo-planar, radial, or unfocused waves) can be transmitted and penetrate soft and hard tissue, fibrotic/scar tissue, blood vessels and body's fluids, which makes them very versatile in treating any type of infections regardless of the location inside the body or type of tissue affected by infection.

Also, acoustic pressure shockwaves or specifically modulated pressure waves (planar, pseudo-planar, radial, or unfocused waves) showed the capability to multiply stem cells form bone marrow, cadmium layer of the bones and to recruit the circulating stem cells from blood for the repair of damaged tissues or organs. That combined with the growth of new small blood vessels and the call-in of different specific growth factors can contribute to tissue regeneration and repair of damaged tissue, which has significant implications in maintaining lung functional capacity and overall oxygenation.

Based on different components of the mechanism of action, as described above, the extracorporeal or intracorporeal acoustic pressure shockwaves and specifically modulated pressure waves (planar, pseudo-planar, radial, or unfocused waves) are capable to play a role in modulating inflammation, immune system response to tone down the cytokine storm surge, and in clearing the respiratory tract of viruses, bacteria, biofilms, micro-organisms, and mucus accumulation, with significant and benefic effects in improving respiration and overall healing. The shortened duration of inflammation known as inflammation modulation that is produced by extracorporeal or intracorporeal acoustic pressure shockwaves and specifically modulated pressure waves (planar, pseudo-planar, radial, or unfocused waves) is reducing the possibility of creating scar tissue inside the lungs.

Also, due to their mechanical action/forces developed by the positive pressures or by the collapse of the cavitational bubbles, the extracorporeal or intracorporeal acoustic pressure shockwaves and specifically modulated pressure waves (planar, pseudo-planar, radial, or unfocused waves) can dislodge the accumulation of mucus, debris, bacteria, viruses, biofilms, etc. inside the lungs and push them in preferred directions (since shockwaves and pressure wave action is uni-directional) towards the central airway, where they can be coughed up, to help with respiration and oxygenation during the period of infection. People who recover after being infected with the novel coronavirus can still be left with substantially weakened lung capacity. Some patients might have around a drop of 20 to 30% in lung function after full recovery. The coronavirus patients' CT scans show “ground glass,” a phenomenon in which fluid builds up in lungs and presents itself as white patches, which can become more pronounced as their illness progressed. This is where the mechanical forces created by the extracorporeal or intracorporeal acoustic pressure shockwaves and specifically modulated pressure waves (planar, pseudo-planar, radial, or unfocused waves) can play a role in eliminating the fluid buildup in lungs, as “ground glass”, by pushing the fluid in preferred directions, to be directed towards the tracheal trunk/central airway for elimination via cough, which in the end can improve the breathing capacity, oxygenation and significantly change in positive direction the long-term outcome for the infected patients.

Furthermore, the extracorporeal or intracorporeal acoustic pressure shockwaves and specifically modulated pressure waves (planar, pseudo-planar, radial, or unfocused waves) are capable of compromise the integrity of the bacterial or viral shell, and thus eradicate them in sufficient number to reduce the infection and allow the body immune system to take care of it easier, without significant complications for the patients. Also, the fragmented pieces of biofilms, bacterium, viruses, or other micro-organisms can expose individual pathogens that were hiding from the immune system, which then can allow the immune system to recognize a pathogen and generate an appropriate natural reaction against it.

Another important action of the extracorporeal or intracorporeal acoustic pressure shockwaves and specifically modulated pressure waves (planar, pseudo-planar, radial, or unfocused waves) is the stimulation of the growth of new small blood vessels for improved healing and call-in of circulatory stem cells and growth factors for the regeneration of the lung tissues. Also, the scar tissue from the lungs can be reduced in size via inflammation modulation and prevention of tissue hyperplasia. Afterwards through revascularization and tissue proliferation, the scar tissue can be revitalized and replaced with new viable cells and tissue, via the healing mechanism produced by the extracorporeal or intracorporeal acoustic pressure shockwaves and specifically modulated pressure waves (planar, pseudo-planar, radial, or unfocused waves).

Due to possibility of the extracorporeal or intracorporeal acoustic pressure shockwaves and specifically modulated pressure waves (planar, pseudo-planar, radial, or unfocused waves) to have variable penetration without any loss of energy through heat (“cold technology”), they are versatile in treating any type of infections regardless of the location inside the lung or type of tissue affected by infection.

Finally, the pathogens when subjected to extracorporeal or intracorporeal acoustic pressure shockwaves or specifically modulated pressure waves (planar, pseudo-planar, radial, or unfocused waves) cannot mutate, as they do in response to a biochemical attack that can generate even more virulence. Thus, in the case of using shockwaves and pressure waves due to their pure mechanical action will prevent any eventual mutation of the pathogen, which decreases the possibility of reinfections.

It is an objective to create devices and modalities to treat the lungs without creating any additional damage. For that, there is a need for carefully tailored shockwaves (focused, unfocused, extracorporeal or intracorporeal) and have multiple modalities to apply the treatment at different energy output personalized based on the specificity of each disease and disease stage. The purpose of the extracorporeal or intracorporeal acoustic pressure shockwaves or specifically modulated pressure waves (planar, pseudo-planar, radial, or unfocused waves) in treating the lungs is fivefold:

Disable infectious pathogens as bacteria, viruses, micro-organisms, etc. and their possible congregation in biofilms, which can reduce the pathogen load that is the cause of the lung infection. Also, by exposing their fragments, the immune system may produce the right components (antibodies, lymphocytes, neutrophils, monocytes, macrophages, complement proteins, cytokines, etc.).

Dislodge the abnormal accumulation of mucus or sputum or fluid produced by the lung afflictions as viral (COVID-19) or bacterial infections (pneumonia), bronchitis, chronic obstructive pulmonary disease (COPD) and its associated chronic bronchitis and emphysema, cystic fibrosis, idiopathic pulmonary fibrosis, etc., which can help with the elimination of the respiratory tract obstruction and improvement in oxygenation.

Modulation of immune system response to a pathogen invading the lungs, via reducing the cytokine storm and modulation/reduction of the inflammatory phase, which also results in reducing the formation of fibrotic/scar tissues inside the lungs with significant implications in improving oxygenation.

Heal the lung tissue via formation/growth of new small blood vessels, reduction of hyperplasia, and call-in of growth factors and circulating stem cells that can help with damaged tissue regeneration and scar elimination.

Prevent any eventual mutation of the pathogen, during the shockwaves or pressure waves treatments, which decreases the possibility of reinfections.

It is a further objective of the present inventions to provide extracorporeal or intracorporeal acoustic pressure shockwaves or specifically modulated pressure waves (planar, pseudo-planar, radial, or unfocused waves) generating devices that are modular, do not need high maintenance and can, if needed, be applied/used in conjunction and synergy with other devices, drugs, methods and existing treatments for treating lung afflictions and infections or for prevention of infections (prophylactic use).

It is another objective of the present inventions to provide different methods of generating focused, unfocused, planar, pseudo-planar or radial extracorporeal or intracorporeal acoustic pressure shockwaves or specifically modulated pressure waves (planar, pseudo-planar, radial, or unfocused waves) for treating lung tissue afflictions and infections using specific devices that contain either of the following acoustic pressure shockwave or pressure wave generators:
electrohydraulic generators using high voltage discharges
electrohydraulic generators using one or multiple laser sources
piezoelectric generators using piezo crystals
piezoelectric generators using piezo fibers
electromagnetic generators using a flat coil
electromagnetic generators using a cylindrical coil
hydraulic generators
pneumatic generators

The devices/systems and embodiments disclosed in this invention, are design in such way that they can direct the extracorporeal or intracorporeal acoustic pressure shockwaves or specifically modulated pressure waves (planar, pseudo-planar, radial, or unfocused waves) in between the intervertebral space to reach the designated area in the lungs.

In the same time some of the devices presented and embodiments disclosed in this invention can have enough energy that they can still penetrate inside the lung region through the vertebra and not only the inter vertebral space. These devices will need to generate more energy at the point of origination, to account for their reflection and thus loss of energy at the bone interface. These comments are valid only for the extracorporeal approach. When the intracorporeal systems are used, they go through tracheal trunk inside the lungs until their dimension is too large to go further at their distal end. These intracorporeal systems do not have any bones interfere with their usage. Also, their big advantage is that they give a more localized treatment at lower energies but with high efficiency.

The amount of energy deposited inside the lung tissue can be adjusted via the input energy settings of the control console, the geometry of the applicator, addition of different substances inside the cavity formed in between the reflector and the membrane of the applicator, frequency of the shockwaves/pressure pulses, or by producing shockwaves or pressure waves off-set from the focal point of the respective geometry (movable spark-gap). From many ways to produce pressure waves (planar, radial, or unfocused waves), the proposed embodiments contemplate the electrohydraulic way to generate them, which can give them a unique control and possible modulation of their output. The reflectors can have special geometry, can be constructed of different materials that produce a more or less efficiency during reflection, and the input energy can be varied/modulated accordingly to the needs via software. Also, some of the applicators are having multiple points of origination of pressure waves, which can produce multiple waves simultaneously or subsequent. Larger reflective surfaces will reflect more energy that smaller surfaces, at similar energy used to originate the shockwaves or the pressure waves.

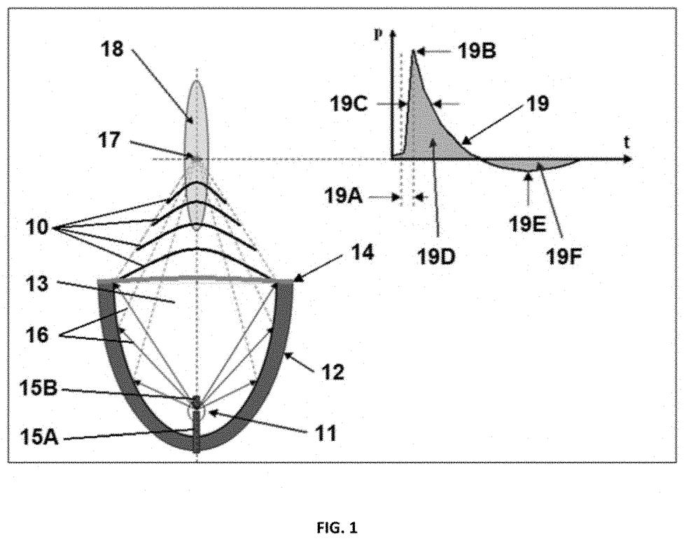

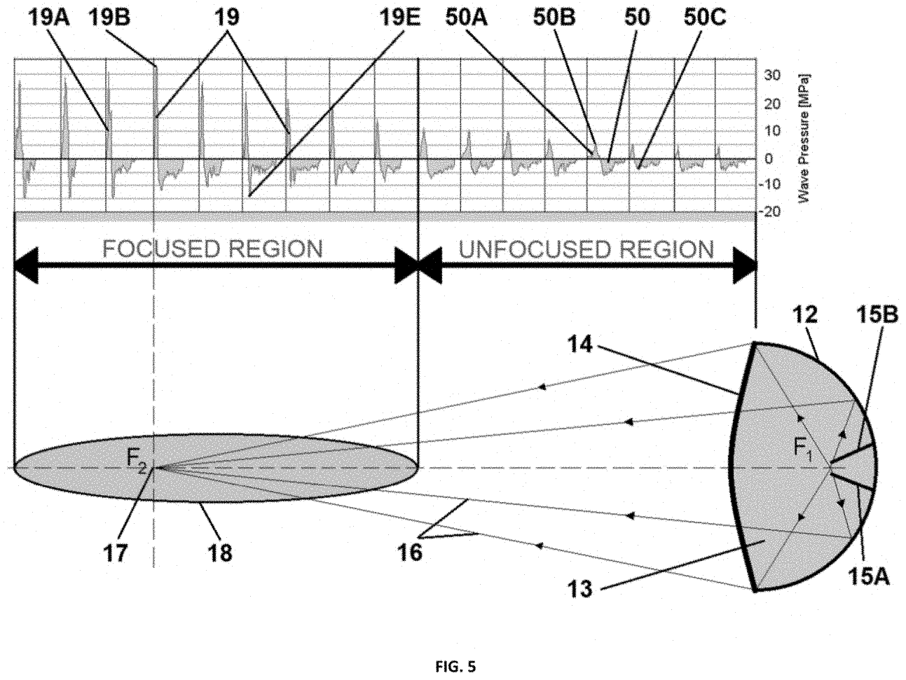

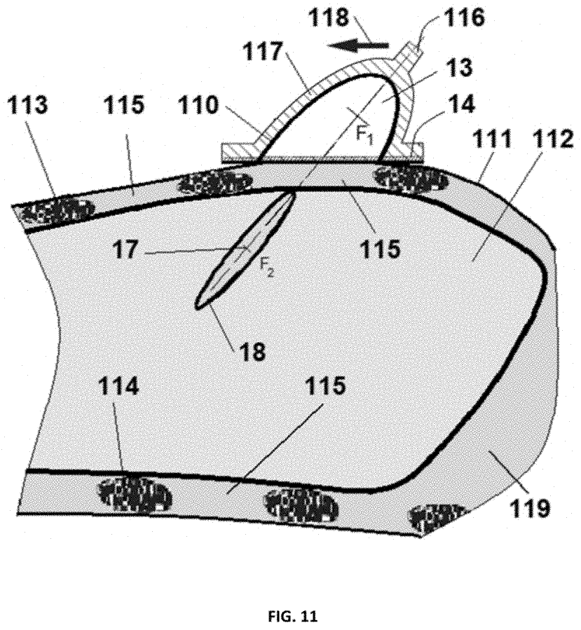

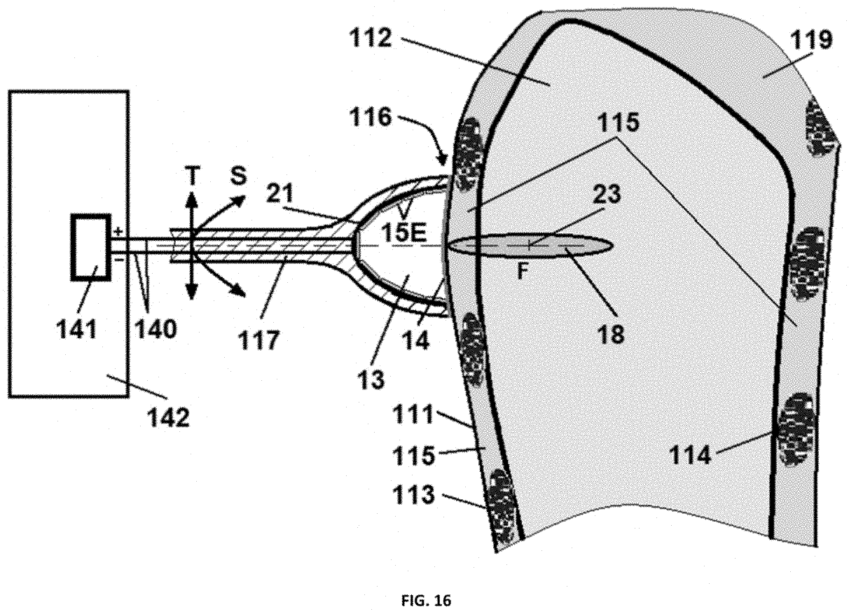

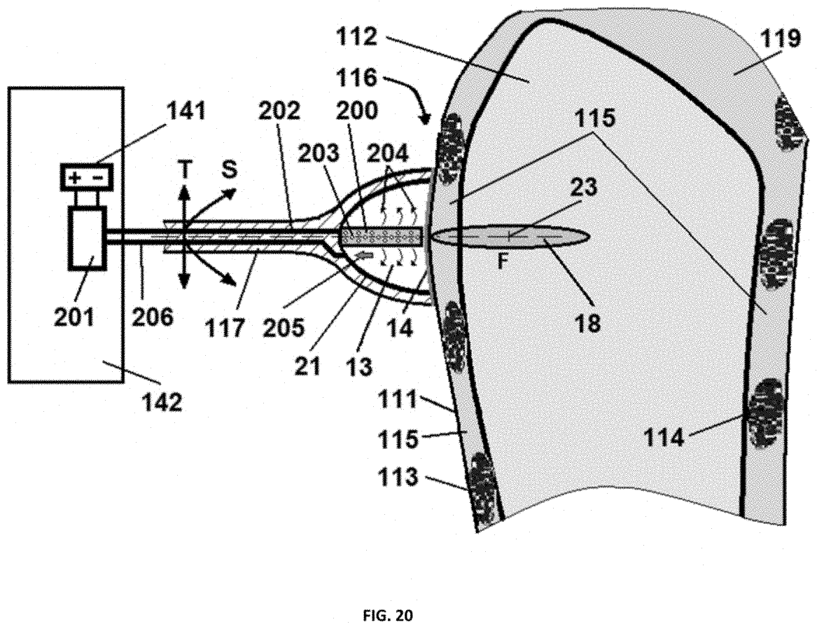

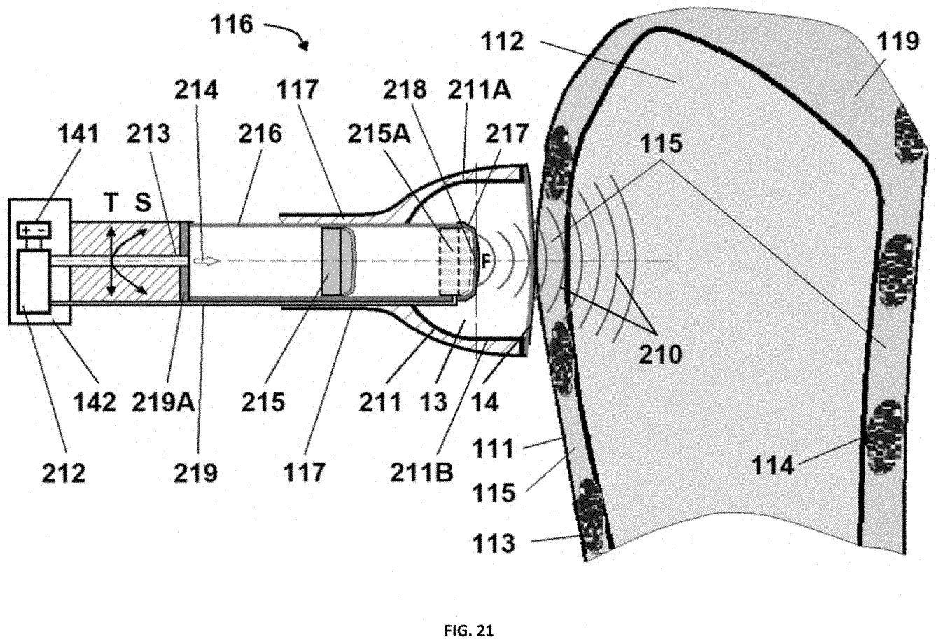

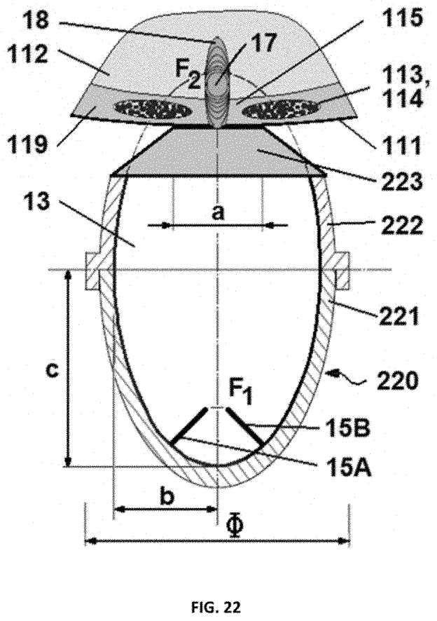

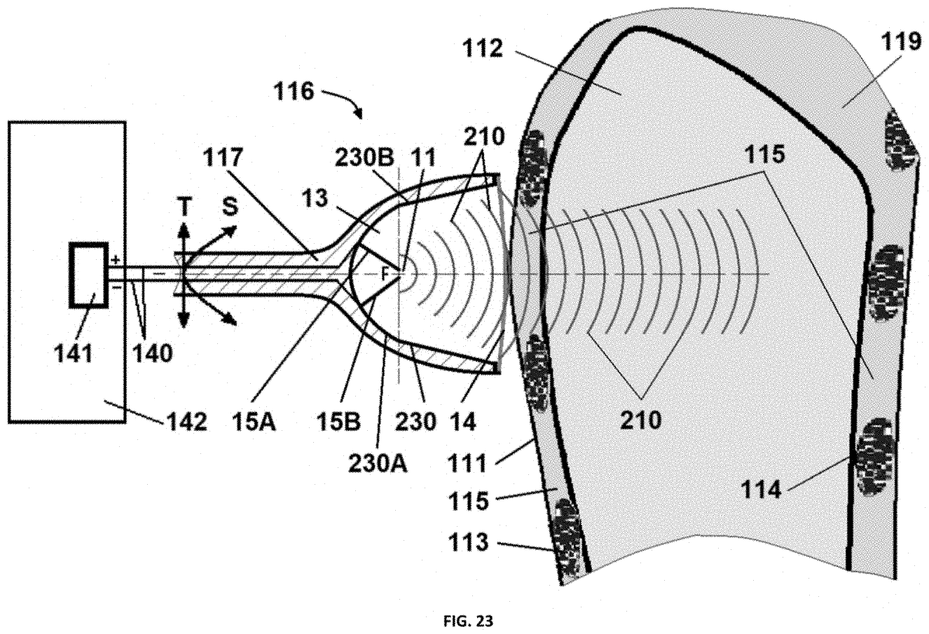

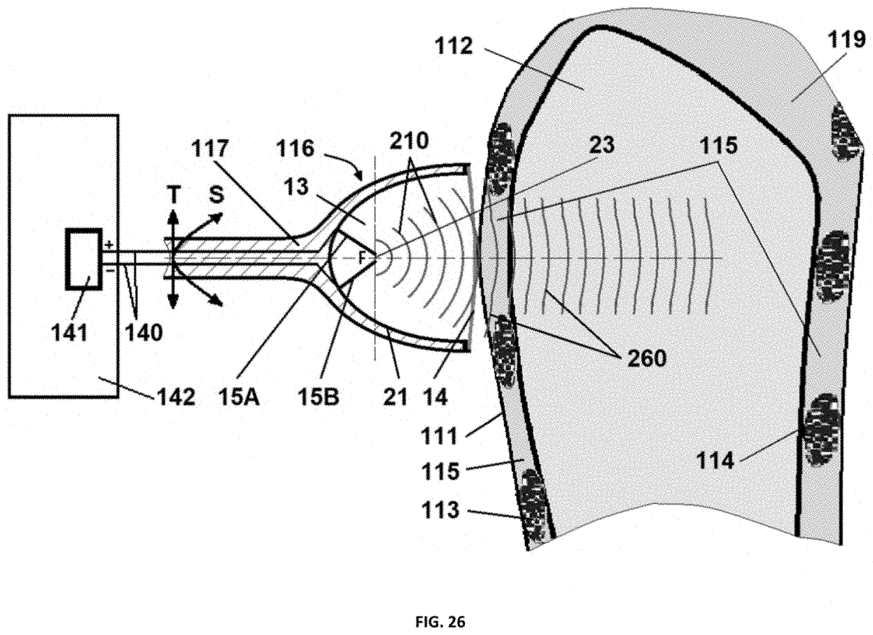

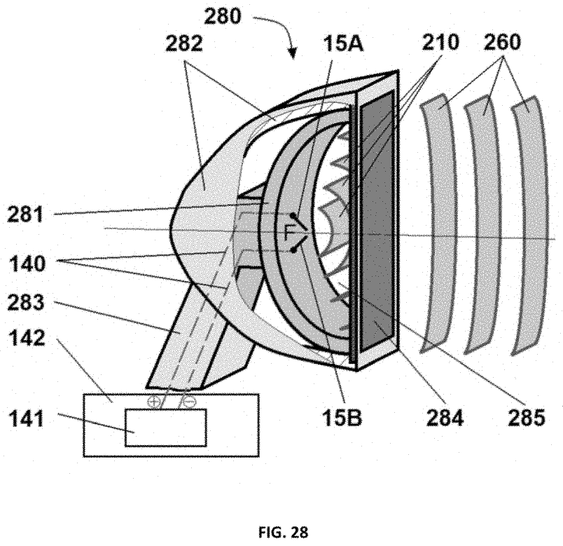

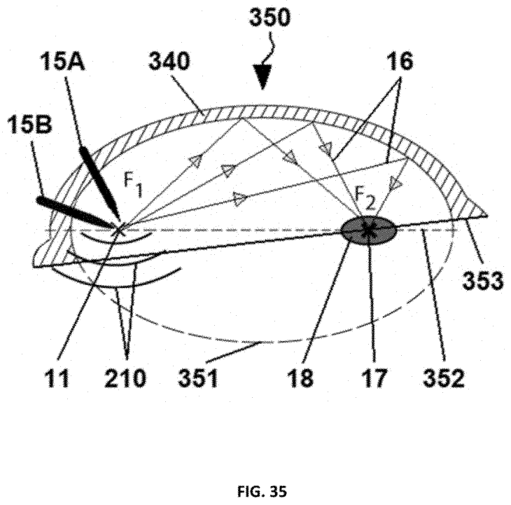

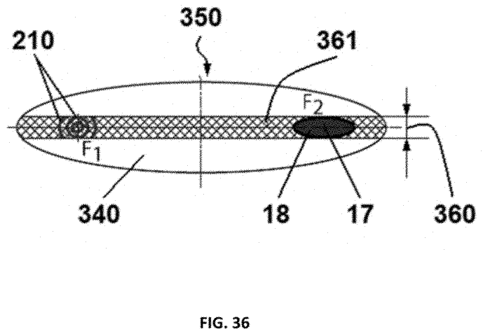

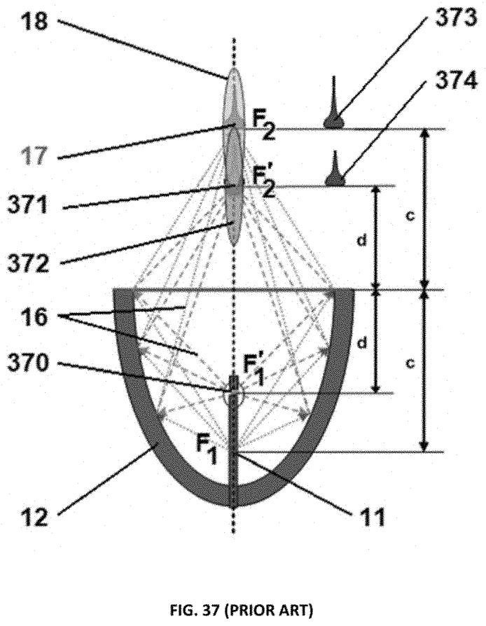

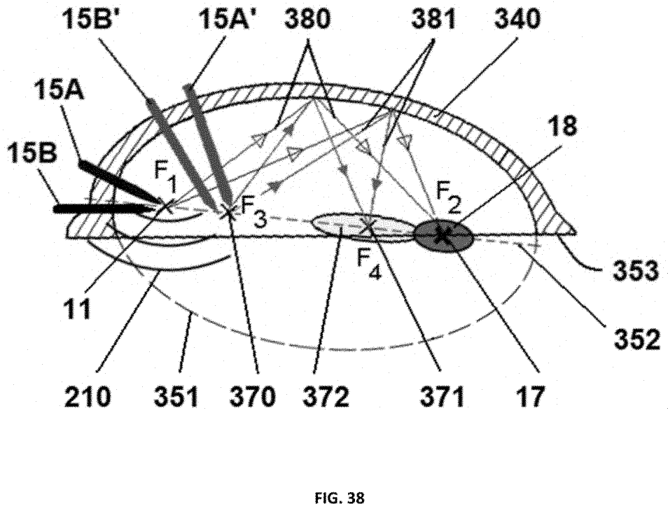

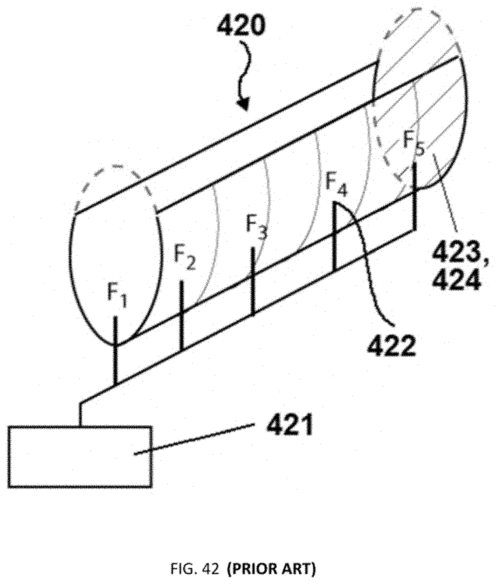

Regarding the reflectors used in the proposed embodiments to treat lung afflictions/diseases or infections, they can be semi-ellipsoidal (see FIGS. 1, 12, 14, 15, 16, 17, 18, and 33) or almost full-ellipsoidal (see FIG. 22) to produce focused shockwave. These reflectors can be shallow or deep to produce superficial or deep penetration and deposit less or more energy inside the tissue. In other situations, an inclined ellipsoidal reflector can be used to allow the penetration of the shockwaves below the ribs, as presented in FIG. 11. For piezoelectric and electromagnetic systems, they use the parabolic reflectors (FIGS. 16, 17, 18, and 19). Spherical reflectors can generate radial pressure waves (see FIGS. 21 and 23) or parabolic reflectors can be used to generate pseudo-planar pressure waves (FIG. 26) or focused shockwaves (FIGS. 16, 17, 18, 19, and 20). The radial and planar pressure waves can be categorized also as unfocused pressure waves. Another way to produce unfocused shockwaves or pressure waves for treating lungs is to place the treatment region before they reach their second focal point of a semi-ellipsoidal reflector, as seen in FIG. 5. Also, unfocused shockwaves or pressure waves can be generated in an offset point from the geometrical focal point (their origination point) of the ellipsoidal reflectors, which could produce a combination of focused and unfocused shockwaves (see FIGS. 37 and 38) or a combination of focused shockwaves and radial pressure waves (see FIGS. 35 and 36). Furthermore, to facilitate the penetration of extracorporeal or specifically modulated pressure waves (planar, pseudo-planar, radial, or unfocused waves) through the vertebrae spaces, segmented reflectors might be used, as presented in FIGS. 28, 29, 30, 31, and 32, or pipe reflectors as seen in FIG. 42. The applicators with segmental reflectors and pipe reflectors will generate less energy, but they can concentrate most of the energy in the intervertebral space, which is sometimes can be desired. In general, the amount of energy that is reflected on the reflector is proportional with its surface. The hydraulic applicators (see FIG. 20) produce pressure waves at smaller scale when the high-pressured hydraulic fluid is exiting through designated small holes out of a central cylinder. A macro pressure wave emanating from the central cylinder is created by the combination of multiple small-scale pressure waves generated by each small hole of the central cylinder. The macro pressure wave of the hydraulic applicator is then reflected towards the targeted region via a parabolic reflector, as seen in FIG. 20. The pneumatic applicator (see FIG. 21) has a cylinder where a bullet is accelerated via pneumatic pressure. When the bullet hits the end-plate create radial pressure waves in the center of a spherical reflector that propagates the radial pressure waves through a fluid towards the contact with the skin and then penetrate the lung. The use of the spherical reflector in combination with the pneumatic cylinder avoids the bruising that might be produce by the end-plate and thus a seamless and much gentler transmission of the radial pressure waves is obtained.

Sometimes combination geometries ca be used for the reflector. Two or more geometries can be used (portion of an ellipsoid, combined with a portion of a sphere and a portion of a paraboloid). That can have an effect on the way the shockwaves or pressure waves are reflected, how many Focal Volumes are created, that can overlap or can be totally separated.

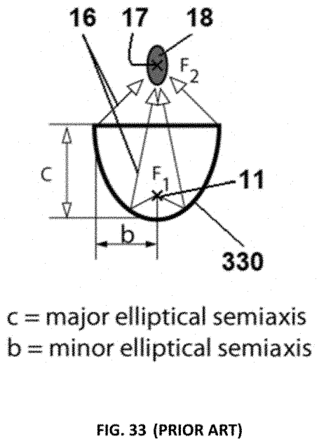

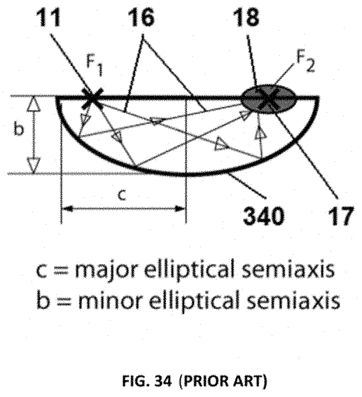

As presented in FIGS. 34, 35, 36, and 38, an interesting option is to use reversed semi-ellipsoidal reflectors (FIG. 34), which have their aperture along the long axis (an ellipsoidal aperture in comparison to the classical approach where the aperture is circular). These reflectors have the following advantages:
A radial pressure wave is generated from F₁ where the high voltage discharge is produced for the spark-gap electrohydraulic principle (see FIGS. 35 and 38), due to the fact that the radial pressure wave propagates below the reflector, since it does not have any surface to bounce back. In this way, the radial pressure waves propagate directly into the body in the treatment area without any reflection on the reversed semi-ellipsoidal reflector.
Focused pressure shockwaves are also present, which are produced by the radial wave generated in F₁ that is reflected by the upper portion of the reversed semi-ellipsoidal reflector and then focused towards second focal point F₂ of the ellipsoidal geometry or focal point 17.
Treatment area is slicing longitudinally the focal volume (FIG. 34) and not transversally as it happening with the classic semi-ellipsoidal reflector design (FIG. 33), which also translates in increased efficiency of the treatment in one fixed position.

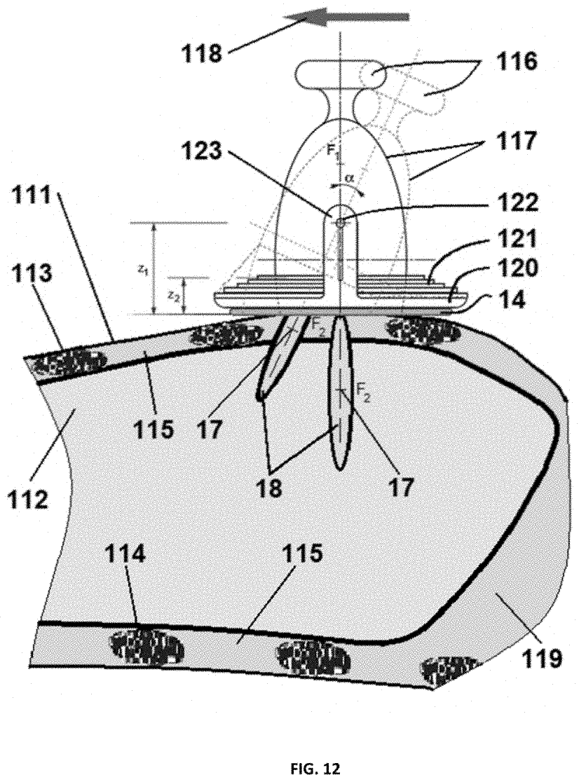

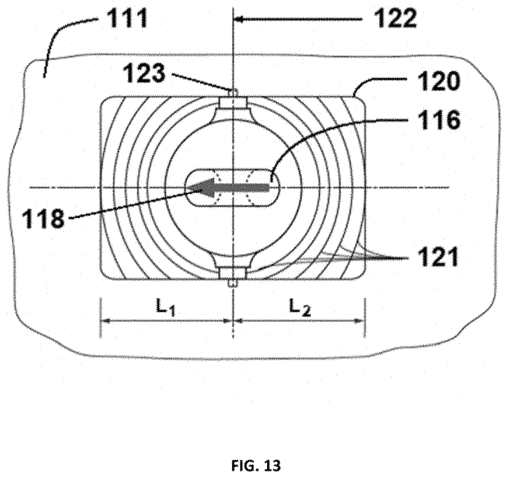

Non-rotational reflector geometries can be also used to reflect shockwaves or pressure waves. In this case, the reflector can have a pyramid geometry with triangle, square, hexagonal, or octagonal aperture. That will create a focusing that is generating a long and almost linear Focal Volumes, that can be a big advantage in penetrating right in between the intervertebral space. The dimensions of the Focal Volumes can be controlled, via reflector's geometry and dimensions, to achieve shallow or deep penetration, through the intervertebral space. Using a design for the handle as the one presented in FIGS. 12 and 13, the direction and penetration of the shockwaves or pressure waves can be adjusted for the lung treatment needs.

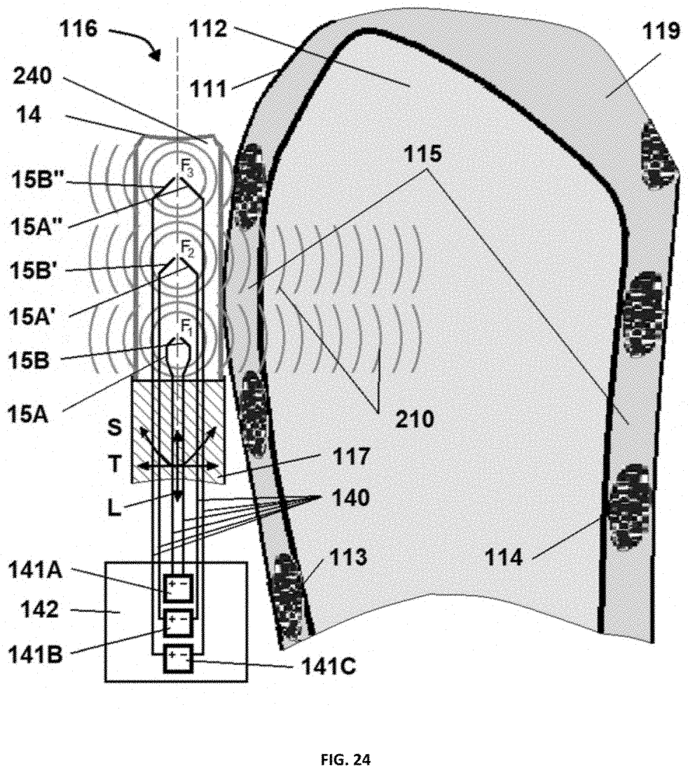

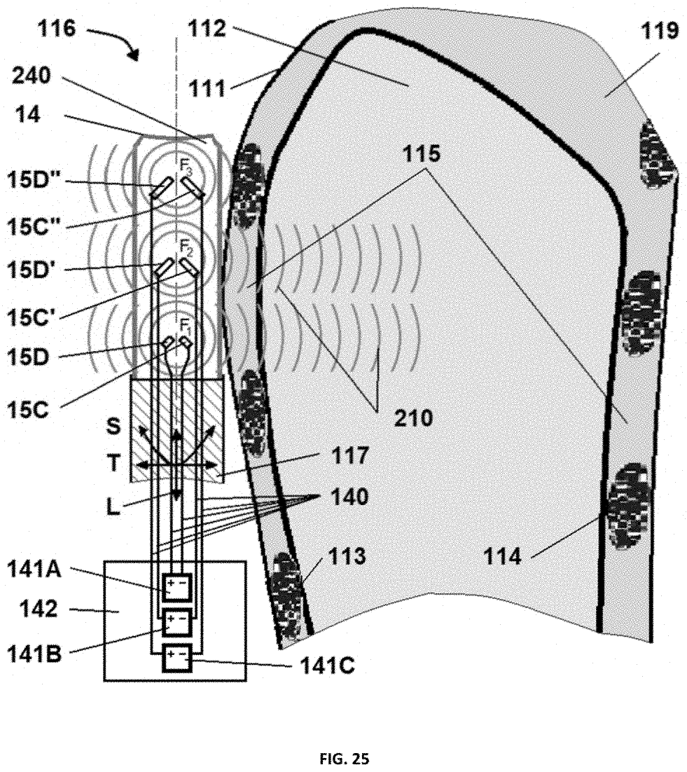

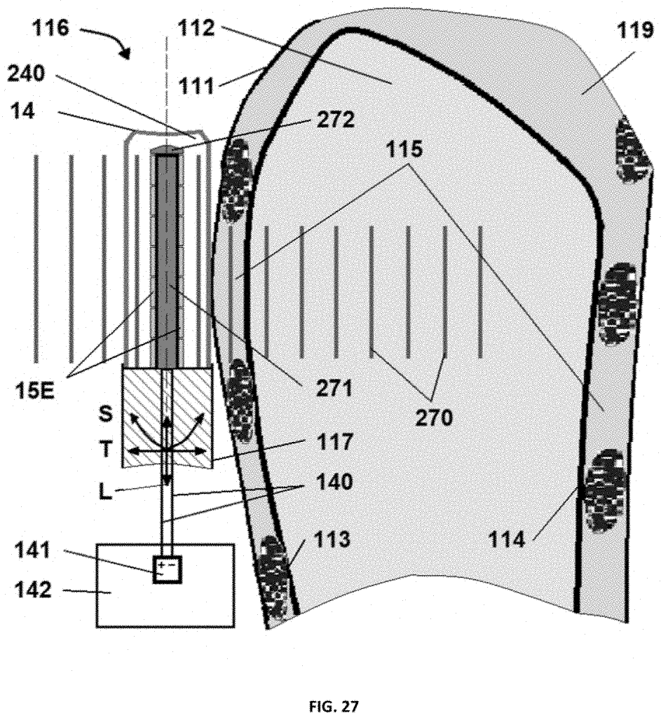

In other situations, no reflectors are used at all, where multiple spark-gap or laser electrohydraulic sources are used (see FIGS. 24 and 25). In this case a radial pressure wave is crated that moves in any direction. However, only the pressure waves that are generated towards the body will travel to the lungs. Another situation in which there is no reflector used is presented in FIG. 27 where piezo-crystals are used to create planar pressure waves via piezo-electric principle. In other embodiments (not specifically shown in FIG. 27) the piezo-crystals can be replaced with piezofibers, which can give more flexibility to accommodate to other substrate surfaces, besides the flat ones, which are characteristic for piezo-crystals.

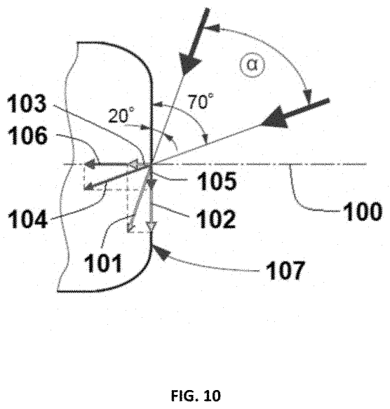

It is important also to generate multiple forces when the shockwaves or pressure waves reach the Focal Volumes or the targeted treatment region. This can be achieved with inclined reflectors (see FIG. 11) that reach on acute angle (20°-70°) relatively to a certain targeted surface (FIG. 10). In this situation, the positive pressures or micro-jets of the collapsing cavitational bubbles can produce force components tangential to the surface and also force components perpendicular to the surface. This can be beneficial in much more efficient cleaning of the airways and lung alveoli from mucus or sputum.

The embodiments of this patent that are used to produce the relieve of symptoms and the healing of the lung common diseases, in general for both human and animals, are further described in detail in the following paragraphs.

FIG. 1 presents the main and unique characteristics for the focused pressure shockwaves, which are the same regardless of the principle used to generate them. In the specific case described in FIG. 1, the shockwaves are produced via the spark-gap electrohydraulic principle. To focus shockwaves, it is necessary to produce them in one point and then focus the shockwaves towards another point where their action is needed. The only geometry that has two focal points is the ellipse and in three-dimensional realm is the ellipsoid (see FIG. 33), which is the geometry used for the reflector 12. However, for the medical field the second focal point F₂ of the ellipsoidal geometry must coincide with the tissue being treated, which means that only a semi-ellipsoidal reflector 12 and not full ellipsoid can be used, as clearly depicted in both FIGS. 1 and 33. Thus, the shockwaves are produced via discharging a high voltage in a fluid from the reflector cavity 13 and between the first electrode 15A and second electrode 15B of the spark-gap 11, which is placed in first focal point F₁ (FIG. 33) of the semi-ellipsoidal reflector 12. The high voltage discharge produces an oscillating plasma bubble that creates kinetic energy in the fluid present in the reflector cavity 13. This high energy kinetic energy is in fact the shockwave that is then reflected by the semi-ellipsoidal reflector 12 towards the second focal point 17 (F₂ from FIG. 33). The shockwave focusing 16 is done towards a focal volume 18 that is centered around the focal point 17 (F₂ of the ellipsoidal geometry). To keep the fluid inside the reflector cavity 13 a coupling membrane 14 is used that stays on top of the opening/aperture of the semi-ellipsoidal reflector 12. In the focal volume 18, produced by the acoustic pressure shockwave 10, there is a special pressure “P” profile function of time “t” that defines the shockwave pressure signal 19. Thus, there is a sharp increase in positive/compressive pressure to the maximum shockwave positive pressure 19B of the shockwave compressive phase 19D, which is defined by a rise time 19A in the range of tens of nano seconds to hundreds of nano seconds. After the positive pressure is reaching the maximum shockwave positive pressure 19B, then the pressure decreases exponentially towards zero, which completely defines the full shockwave compressive phase 19D of the shockwave pressure signal 19. The pulse width 19C is defined as the time interval beginning at the first time the positive pressure exceeds 50% of the maximum shockwave positive pressure 19B. The larger the pulse width 19C, the larger and more powerful is the shockwave compressive phase 19D and its influence on the lung tissue, to produce mucus dislodging, elimination of infection and overall healing. Once the pressures are in the negative values, they are in the shockwave tensile phase 19F. After reaching the maximum shockwave negative pressure 19E, the pressure is going back towards zero to completely outline the shockwave tensile phase 19F profile and also define the end of the shockwave pressure signal 19. The total time duration of a shockwave pressure signal 19 is in between five to eight microseconds, which defines a strong and rapid pressure variations, that produces significant, controlled, and efficient effects on the lung tissue, with the ultimate effect being elimination of infection and tissue healing.

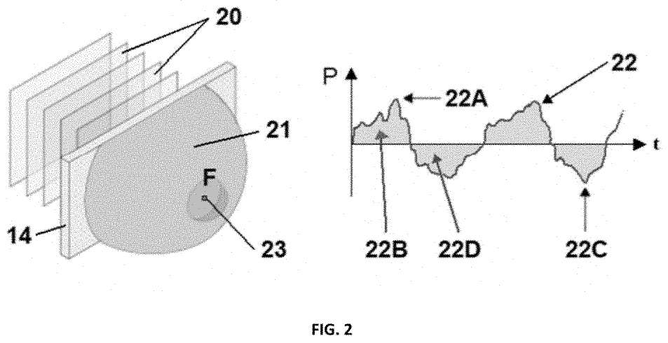

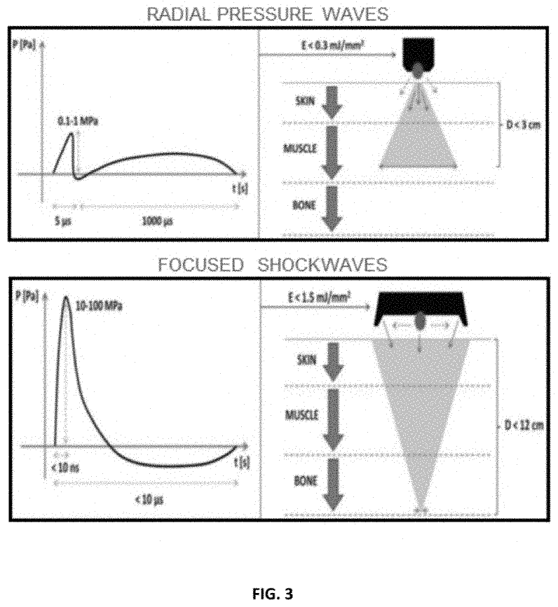

Shockwaves are more powerful in general and deposit more energy in the targeted tissue when compared to pressure waves, which are having a pressure signal flatter and more sinusoidal in shape for planar (FIG. 27) or pseudo-planar waves (slightly distorted planar waves), as presented in FIGS. 2, 26, 28, 29, 30, 31, and 32, and distorted tooth-shape towards the positive pressures for radial waves, as seen in FIG. 3. Due to lower positive pressures and smaller values for negative pressures for the planar waves/pseudo-planar waves or radial waves, such pressure waves will put less energy inside the treatment zone, when compared to shockwaves. However, sometimes the lower energy is desired to avoid any negative impact on the delicate tissues from the lung that is affected by a certain disease. The acoustic planar/pseudo-planar pressure waves 20 (FIG. 2) are characterized by almost equal maximum planar wave positive pressure 22A and maximum planar wave negative pressure 22C in absolute values, which makes the planar wave pressure signal 22 to have nearly a sinusoidal shape/variation for pressure “P” versus time “t”. This also means that the planar wave compressive phase 22B and the planar wave tensile phase 22D have similar energy incorporated in them (given by the area under the curve and time axis). The reflectors used to create acoustic planar/pseudo-planar pressure waves 20 are parabolic reflectors 21 characterized by only one focal point known as parabolic focal point 23 (F), as presented in FIGS. 2, 26, 28, 29, 30, 31, and 32. The pressure waves are generated by the high voltage discharge in between the first electrode 15A and second electrode 15B of the spark-gap 11 placed in the parabolic focal point 23 (F), as presented in FIGS. 2, 26, 28, 29, 30, 31, and 32. The pressure waves are moving radially from the parabolic focal point 23 (F) in the form of acoustic radial pressure wave 210 (see FIG. 26) towards the parabolic reflectors 21, which reflects all the waves parallel to its longitudinal axis (similar to a flash light) and thus creating outside the reflector the acoustic planar/pseudo-planar pressure waves 20 (FIG. 2). In some cases, clean acoustic planar pressure waves 270 are created using planar piezoelectrical crystals as presented in FIG. 27.

The comparison of wave forms, pressure values, the energy deposited in the treatment zone measured as energy flux density (mJ/mm²), in between acoustic radial pressure waves and focused acoustic pressure shockwaves is presented in FIG. 3. Besides the significant difference in the shape of the pressure wave forms, it can be noticed that the acoustic radial pressure waves have a duration (more than one thousand microseconds), which is more than 100 times longer when compared to shockwaves (less than ten microseconds, or more precise in between five to eight microseconds). The maximum positive pressures for shockwaves can be 10 times to maximum 1000 times higher when compared to radial pressure waves. The limitation for radial pressure waves is given by the fact that the highest pressure is generated at the patient's skin and to not produce hematomas or discomfort, the pressures cannot be higher than 1 MPa (10 bar). The energy deposited inside the tissue in an area through which the shockwaves or radial pressure waves have passed, is 5 times higher for shockwaves. Also, the penetration for shockwaves is 4 times deeper when compared to radial pressure waves. Finally, the shockwaves are capable of treating all kind of tissue from soft (skin and muscle), semisoft (tendons and ligaments), to hard (bones), which is more extensive than the radial pressure waves that can only treat soft tissue. Once inside the body, the radial pressure waves lose their energy pretty fast, and thus the limited penetration and power that allows them to mainly treat soft tissues. All these parameters show indeed that the acoustic pressure shockwaves are more powerful in general and deposit more energy in the targeted tissue when compared to acoustic radial pressure waves.

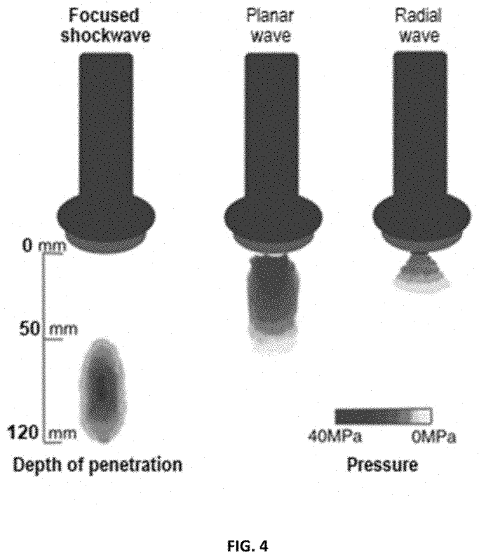

FIG. 4 clearly shows the differences in between focused shockwaves, the planar/pseudo-planar pressure waves, and radial pressure waves. The penetration of the shockwaves can be up to 120 mm, and they can be focused anywhere from superficial to deep inside the human or animal body. The planar/pseudo-planar pressure waves start at the skin level and can penetrate up to 50 mm in unfocused way. However, the planar/pseudo-planar pressure waves can develop pressures up to 20 MPa (200 bar), which are 5 times less than the maximum pressures produced by the focused pressure shockwaves for medical purposes. Although, for the lung treatment lower pressures might be used, which can be in the same ballpark of the ones generated by the planar/pseudo-planar pressure waves. The radial pressure waves have the lowest penetration of maximum 3 cm, and have the smallest maximum pressures (no higher than 1 MPa/10 bar) as discussed and seen in FIG. 3. These significant differences dictate the choice of shockwaves or pressure waves for lung treatment and the medical approach to deliver properly and efficiently different types of shockwaves/pressure waves for lung therapy.

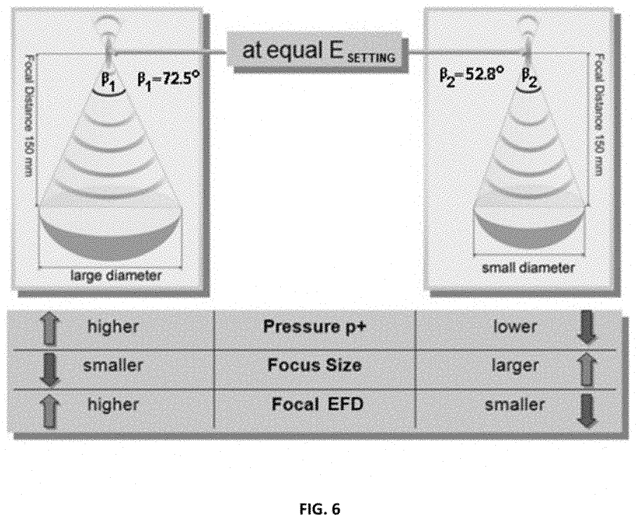

As is seen in FIG. 5, there is also a significant difference in between focused and unfocused acoustic pressure shockwaves that were produced inside a semi-ellipsoidal reflector 12, via the high voltage discharge in the reflector cavity 13 filled with a liquid and in between first electrode 15A and the second electrode 15B. Shockwaves are then reflected on the semi-ellipsoidal reflector 12 and the shockwave focusing 16 is produced that directs/focuses the shockwaves towards the focal point 17 (F₂ of the ellipsoidal geometry) and focal volume 18. The Unfocussed Region could be also named Focusing Region. The Unfocused Region runs from the semi-ellipsoidal reflector 12 edge up to the focal volume 18 that encompasses the geometrical focal point 17 (F₂). This is the region where the shockwaves focusing 16 towards focal point 17 (F₂ of the ellipsoidal geometry) is produced, which transforms the sinusoidal wave generated in F₁ (the first focal point of the ellipsoid where spark-gap 11 is centered—see FIG. 1) in strongly distorted and characteristic shockwave pressures signals 19 from the focal volume 18. In the Unfocussed Region the unfocused shockwave pressure signals 50 have different shape and characteristics when compared to the shockwave pressures signals 19 from focal volume 18 that defines the Focused Region. Thus, it is clear that there is significantly slower unfocused shockwave rise time 50A for the unfocused shockwave pressure signals 50, which shows that less powerful shockwaves are found in the Unfocused Region. The same is indicated by much lower unfocused shockwave maximum positive pressure 50B and significant smaller absolute value for unfocused shockwave maximum negative pressure 50C. The Focused Region is the region where the shockwaves are focused and where the largest pressures and energies are created. The extend of the Focused Region is defined by the length of the focal volume 18 and it is the region where the acoustic pressure shockwaves 10 (FIG. 1) have the characteristic shockwave pressure signal 19 with very fast rise time 19A, high maximum shockwave positive pressure 19B, and larger maximum shockwave negative pressure 19E, which translates in higher energy that is incorporated into these focused shockwaves. This allows deeper penetration and the capability to pass through significantly obstacles, as the ribs, where a part of the energy is lost due to reflection on the bone surface, but still have enough energy transmitted through the bone and into the lung tissue, to have the desired therapeutic effect. In conclusion, such focused shockwaves can be transmitted through the ribs, which simplifies the construction of the shockwave applicator and allows the use of a larger applicator that covers in one fixed position a greater area/region inside the lung. Overall, the larger and more powerful applicators generating focused shockwaves will cover both lungs in a faster time, making the overall treatment shorter. However, the energy of the focused shockwaves can be also adjusted based on the input energy settings (the value of energy used to create shockwaves at the point of their origin), which will give a wide range of energy choices, from high to medium, that can be tailored for each specific type of disease or infection of the lungs. Furthermore, the level of pressures and energy flux density produced in the focal zone can be also modified or adjusted or modulated through the reflector's geometry (dimensional characteristics), as presented in FIG. 6, the angle of the longitudinal axis of the reflector relatively to the surface or region to be treated (see FIGS. 10, 11, and 12), or by changing the area of reflective surface of the reflector (see FIGS. 22, 28, 29, 30, 31, and 32), or by slicing the reflector along the longitudinal axis instead of transversal axis (see FIGS. 34, 35, 36 and 38), or by having variable/movable origination point for the shockwaves, as presented in FIGS. 37 and 38. FIG. 6 shows for the focused shockwaves the influence of the diameter of the opening of the semi-ellipsoidal reflector 12, also known as aperture. For a certain energy input setting (E_{SETTING}), a larger reflector diameter at the aperture, will create higher positive pressures and corresponding energy flux density (EFD) values in the treatment region. However, due to focusing from a larger reflector surface and different angle of focusing (β) the focusing is done in a smaller focal volume (length and diameter). Conversely, smaller reflector diameter creates a larger focal volume, but lower positive pressures and corresponding energy flux density (EFD) values in the treatment region. All the other ways to vary the energy output of the focused shockwaves will be presented later in detail when these embodiments will be discussed in association with their representative figure or figures.

On sharp contrast to the focused shockwaves, by placing the lung into the Unfocused Region (as presented in FIG. 5), the unfocused shockwave energy can be modulated or reduced to the level of planar, pseudo-planar, or radial pressure waves. The use of lower energies delivered by the unfocused shockwaves can be the approach for milder treatments or for the case when the unfocused shockwaves are transmitted through the intervertebral space, without any attenuation from the ribs. Lower energies deposited by the shockwaves inside the lung tissue reduces the possibility of side effects, as damage to the of the airways/tracheal trunk and lung alveoli.

In FIG. 7 is described the mechanotransduction process that is generated by the variation of pressure outside bacterium outer wall, which can lead to changes in bacterial turgor hydrostatic pressure that can produce bacterial cell destruction. Turgor hydrostatic pressure is the force within a cell that pushes the plasma membrane against the cell wall. Generally, turgor pressure is caused by the osmotic flow of water in and out of the cell and occurs in plants, fungi, and bacteria. This system is not seen in animal cells. Osmotic flow of water through a semipermeable membrane is happening when the water travels from an area with a low-solute concentration, to one with a higher-solute concentration. The pressure exerted by the osmotic flow of water is called turgidity. The semipermeable bacterial membrane is given by the presence of mechanosensitive (MS) channels, as seen in the inset that shows a MS channel in a Gram-negative bacterium. In general, bacterial cells accumulate sufficient solutes inside them to achieve an outwardly directed turgor pressure of ˜4 atm, which is balanced by the resistance of the bacterium wall and outer membrane. Upon hyperosmotic shock (high osmotic pressure outside the bacterium outer wall), the bacterial cell reduces its internal pressure by losing water and shrinking. After the hyperosmotic shock they recover by the accumulation of more compatible solutes. Under these conditions, the MS channels remain closed. If a low osmolarity environment or hypoosmotic shock (low osmotic pressure outside the bacterium outer wall) is created subsequently to the hyper osmotic shock, it leads to the rapid entry of water accompanied by the immediate activation of the MS channels. When MS channels open, they create large pores that allow hydrated solutes to pass through freely, which results in the loss of low molecular mass solutes but the retention of large proteins and solutes. This enables a rapid decrease in the cytoplasmic concentrations of osmotically active solutes and lowers the osmotic driving force for water entry. The influx of water bloats the bacterial cell and generates high turgor pressure that pushes hard the cytoplasmic membrane against the cell wall, which on millisecond to second timescale, has limited capacity to stretch. The rise in turgor pressure results in deformation of the cytoplasmic membrane, and consequently the strain on the bacterial cell wall is increased. If this strain is not relieved it can lead the lysis of the bacterial cell when the pressure exceeds the mechanical strength of the bacterial cell wall (FIG. 7). According to the published scientific literature, the bacterial membrane mechanotransduction occurs at pressures of approximately 3 MPa or less, which is exactly in the realm of pressure generated by acoustic pressure shockwaves or specifically modulated pressure waves (planar, pseudo-planar, radial, or unfocused waves). Furthermore, the bacterium membrane mechanosensitive channels (MS) or pores remain opened due to sudden and continuous pressure changes and the bacteria swells until the integrity of the membrane is compromised. Also, fairly new research showed that the shockwaves and pressure waves are capable to open vesicles in lipid membranes for enveloped viruses. Once the lipid viral envelop is destroyed the enveloped viruses are rendered harmless, since they are no longer able to recognize sites to identify and attach the host cells. For the non-enveloped viruses, the external coat or envelop called capsid is made of proteins. The capsid of the non-enveloped viruses is vulnerable to the mechanical forces and stresses produced by the shockwaves and pressure waves, or to the action of free radicals, and also to the transient sonoluminescence and heat produced during collapse of the cavitation bubbles generated in the tensile phase by the negative pressures. The action on bacteria and viruses of the extracorporeal and intracorporeal acoustic pressure shockwaves or specifically modulated pressure waves (planar, pseudo-planar, radial, or unfocused waves) demonstrate their role in treating bacterial and viral infections of the lungs.

FIGS. 8 and 9 show the structure of the lungs and what it happens when the lung is affected by viral infections, bronchitis, pneumonia, chronic obstructive pulmonary disease (COPD), idiopathic pulmonary fibrosis (IPF), or cystic fibrosis, to name a few.

It is important and the objective of this invention to provide a variety of novel acoustic pressure shockwave applicator constructions for treating lung tissue, determined by the specific reflector shape, and their capability to guide or focus acoustic pressure shockwaves on a specific direction, as schematically is shown in FIG. 10. The acoustic pressure shockwaves direction relative to the surface of the lung anatomic feature 107 plays an important role in the way the action of the acoustic pressure shockwaves is applied during medical treatment of the lung. According to FIG. 10, the direction used for focusing the acoustic pressure shockwaves should be done at an optimal angle α in between 20°-70° relative to the surface of the lung anatomic feature 107. In this way the pressure/force produced by acoustic pressure shockwaves on the surface of the lung anatomic feature 107 splits in two force components. One pressure/force component will be tangential to the surface of the lung anatomic feature 107 and the other pressure/force component will be perpendicular to the surface of the lung anatomic feature 107, along the axis perpendicular to animal meat/carcass surface 100. Relatively to the surface of the lung anatomic feature 107, the 20°-angle shockwave pressure/force 101 splits in the 20°-angle shockwave pressure/force tangential component 102 and the 20°-angle shockwave pressure/force perpendicular component 103. Correspondingly, the 70°-angle shockwave pressure/force 104 splits in the 70°-angle shockwave pressure/force tangential component 105 and the 70°-angle shockwave pressure/force perpendicular component 106. When the pressure/force components' actions are analyzed for each direction, interesting conclusions can be drawn. The tangential pressure/force component along the surface of the lung anatomic feature 107 can help with displacement or scrapping of the mucus or possible bacterial biofilms from the respective lung anatomic feature 107. The 20°-direction relatively to the surface of lung anatomic feature 107 can create greater tangential force component when compared to the 70°-direction. The normal or perpendicular pressure/force components acting perpendicular to the surface of lung anatomic feature 107 can penetrate inside of it and treat the whole feature (for example an alveolus). In this case, the 20°-direction relatively to the surface of the lung anatomic feature 107 can create smaller perpendicular force component when compared to the 70°-direction. The cavitational micro-jets produced by the cavitational bubbles will also be directed towards the surface of the lung anatomic feature 107, which due to their sub-millimeter action will be able to also to displace or scrap the mucus or possible bacterial biofilms from the respective lung anatomic feature 107. The combined action of tangential force component, normal force component, and cavitational jets will ensure a thorough medical action on the lung anatomic feature 107. Furthermore, as seen in FIGS. 11, 12 and 13, the inclined direction of action of the focused or unfocused shockwaves can allow the treatment of the targeted lung tissue 112 under the ribs with an extracorporeal applicator 116 that directs the shockwaves in the intervertebral space 115 of the ribs (FIG. 11). Depending on each specific lung treatment situation, the direction of the acoustic pressure shockwaves can be set at different angles or in other situations can be continuously moving in between 20 and 70 degrees. The angle change can be accomplished by employing a manual or automatic swiveling motion “S” of the acoustic pressure shockwaves around a fixed point (see embodiment from FIGS. 12 and 13).

In FIG. 11 is presented an embodiment of this invention that uses an inclined semi-ellipsoidal reflector 110 to direct the shockwaves at an optimal angle α in between 20°-70° relatively to the surface of the lung anatomic feature 107 (see FIG. 10). This allows the treatment of the targeted lung tissue 112 from under the frontal portion of vertebra/rib 113 with an extracorporeal applicator 116 that directs the shockwaves in the intervertebral space 115 of the ribs. In other situations, the targeted lung tissue 112 from under the posterior portion of vertebra/rib 114 will be treated with the applicator 116 by directing the shockwaves in the intervertebral space 115 of the ribs with the applicator 116 positioned on the back of the patient body 119 (human/animal). For this embodiment, the inclined semi-ellipsoidal reflector 110 sits inside the applicator enclosure/housing 117 that is used by the medical professional to move the applicator 116 in different directions via the applicator translation movement 118, to completely cover the targeted lung tissue 112 that needs treatment with acoustic pressure focused shockwaves or specifically modulated pressure waves (that can be planar, pseudo-planar, radial, or unfocused waves). The same principle and similar extracorporeal applicator 116 construction can be applied for the specifically modulated pressure waves (that can be planar, pseudo-planar, radial, or unfocused waves), where a parabolic reflector 21 (FIGS. 2 and 26), or segmented parabolic reflector 281 (FIGS. 28, 29, 30, 31, and 32), or combination semi-spherical and cylindrical reflector 211 (FIG. 21), or combination semi-spherical and conical reflector 230 (FIG. 23), or reversed semi-ellipsoidal reflector 340 (FIGS. 34, 35, 36 and 38) are used in the construction of the extracorporeal applicator 116. The smooth contact of the applicator 116 with the skin 111 is done via applicator/coupling membrane 14 that can accommodate the normal curvature of the patient's body. To proper transmit the acoustic pressure focused shockwaves or specifically modulated pressure waves (that can be planar, pseudo-planar, radial, or unfocused waves) through the skin 111 and into the targeted lung tissue 112, an ultrasonic gel, or an aquagel, or a hydrogel, or a coupling viscous fluid is used in between the skin 111 and the applicator/coupling membrane 14 (not specifically showed in any of the figures of this invention). For this type of applicators 116 it is preferable to have their action in between intervertebral space 115, where the shockwaves or pressure waves are freely penetrating the targeted lung tissue 112 without any energy loss on obstacles in their way, as the ribs. However, when shockwaves or pressure waves hit the ribs they will bounce back on the bone and a smaller portion of the initial energy of the shockwaves or pressure waves will be transmitted inside the lung tissue. This means that it is possible to also deliver reduced energy through the frontal portion of vertebra/rib 113 or through the posterior portion of the vertebra/rib 114 (through the ribs in general), depending on the frontal or posterior location of the applicator 116 during the targeted lung tissue 112 treatment. Other option is to have these applicators 116 with overall smaller dimension to be able to fit the shockwaves or pressure waves only in the intervertebral space 115. To further help, for the smaller applicators 116, an applicator/coupling membrane 14 can be used that has a special shape, as conical or a narrow membrane, to match at the contact with the skin 111 the dimensions of the intervertebral space 115, as seen in FIGS. 22 and 36.

Conversely, if for FIG. 11 an inclined parabolic reflector (not specifically shown in FIG. 11) is used instead of an inclined semi-ellipsoidal reflector 110, unfocused pressure waves are generated, which create outside of the applicator/coupling membrane 14 of the applicator 116 a pressure field that overlaps with the targeted lung tissue 112 volume to produce the desired treatment of lung affliction/disease or infection. Based on the needs for the treatment of each specific lung a certain solution can be used to produce more or less energy in the targeted lung tissue 112, which offers a lot of flexibility for the medical personnel performing the treatment.

To direct the shockwaves or specifically modulated pressure waves (that can be planar, pseudo-planar, radial, or unfocused waves) at an optimal angle α in between 20°-70° relative to the skin 111, in certain embodiments, it is beneficial if an applicator 116 can be created with adjustable angle in relation to the patient body 119 (human/animal). This will allow the treatment of the targeted lung tissue 112 at different depths and directions for the shockwaves or specifically modulated pressure waves (that can be planar, pseudo-planar, radial, or unfocused waves). As shown in FIGS. 12 and 13, this approach can be realized by sitting the applicator 116 on a hinge 123 that is part of an external frame 120. The bellows 121 are deigned to connect the external frame 120 with the applicator enclosure/housing 117 to keep an enclosed volume of fluid inside the applicator 116 to help with propagation of shockwaves or specifically modulated pressure waves (that can be planar, pseudo-planar, radial, or unfocused waves) without energy loss inside the patient body 119 (human/animal). For the embodiment presented in FIGS. 12 and 13, since the bellows 121 are constructed to fit inside the rectangular external frame 120 and connect to the rotational geometry of the applicator 116, the bellows 121 for this embodiment are circular in shape. To match the rotational shape of the applicator 116 an optimal shape for the external frame 126 might be circular, which is different from the solution presented in FIGS. 12 and 13. However, the external frame 120 and associated bellows 121 can have any other possible shape, as round/circular, oval, rectangular, diamond, square, elliptical, etc. With continuing reference to FIG. 13, the external frame 120 has a rectangular shape that is longer on the direction of longitudinal movement 118, relatively to the position of the applicator 116 on the frame 120 (respectively L₁>L₂). This allows the physician to have a better visual assessment for the position of the applicator 116 on the targeted area of the skin 111, to properly transmit the shockwaves or specifically modulated pressure waves (that can be planar, pseudo-planar, radial, or unfocused waves) in the targeted lung tissue 112, based on intended treatment needs. Yet, the L₁ and L₂ can be qual in other embodiments.

By means of inclining the applicator 116 the focal volume 18 is positioned in different directions and at different depths inside the targeted lung tissue 112, as can be seen in FIG. 12. Furthermore, the pivotal axis 122 provided by the hinge 123 can be moved vertically in between Z₁ and Z₂. In this way penetration depth can additionally be adjusted and the position of the focal point 17 (F₂ of the ellipsoidal geometry) of the focal volume 18 can be adjusted inside the targeted lung tissue 112. As shown in FIG. 12, by rotating the applicator 116 around the pivotal axis 122 a different portion of the targeted lung tissue 112 can be treated, such as a superficial or deep. This rotational movement combined with adjustable position for the pivotal axis 122 in between Z₁ and Z₂, can give multiple treatment options to physicians, depending on the specific affliction of the lung. The same principle and similar extracorporeal applicator 116 construction can be applied for the specifically modulated pressure waves (that can be planar, pseudo-planar, radial, or unfocused waves), where a parabolic reflector 21 (FIGS. 2 and 26), or segmented parabolic reflector 281 (FIGS. 28, 29, 30, 31, and 32), or combination semi-spherical and cylindrical reflector 211 (FIG. 21), or combination semi-spherical and conical reflector 230 (FIG. 23), or reversed semi-ellipsoidal reflector 340 (FIGS. 34, 35, 36 and 38) are used in the construction of the extracorporeal applicator 116. As presented in FIG. 12, for this type of applicators 116 it is preferable to have their action in between intervertebral space 115, where the shockwaves or pressure waves are freely penetrating the targeted lung tissue 112 without any energy loss on obstacles in their way, as the ribs. However, when shockwaves or pressure waves hit the ribs they will bounce back on the bone and a smaller portion of the initial energy of the shockwaves or pressure waves will be transmitted inside the lung tissue. This means that it is possible to also deliver reduced energy through the frontal portion of vertebra/rib 113 or through the posterior portion of the vertebra/rib 114 (through the ribs in general), depending on the frontal or posterior location of the applicator 116 during the targeted lung tissue 112 treatment. Other option is to have these applicators 116 with overall smaller dimension to be able to fit the shockwaves or pressure waves only in the intervertebral space 115. To further help, for the smaller applicators 116, an applicator/coupling membrane 14 can be used that has a special shape, as conical or a narrow membrane, to match at the contact with the skin 111 the dimensions of the intervertebral space 115, as seen in FIGS. 22 and 36.

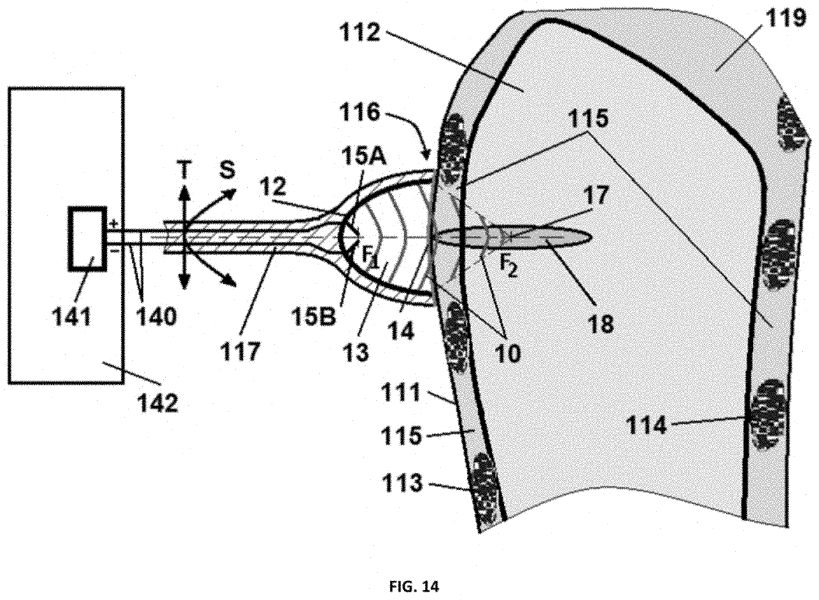

In FIG. 14 the focused acoustic pressure shockwaves 10 are generated via high voltage discharge produced in between first electrode 15A and the second electrode 15B (electrohydraulic principle using spark gap high voltage discharges) in a fluid present inside the reflector cavity 13. The high voltage for the first electrode 15A and the second electrode 15B is provided by the power supply 141 (included in control console/unit 142) via high voltage cable 140. The two electrodes 15A and 15B are positioned in the first focal point F₁ (forming the spark-gap 11, as presented in FIG. 1) of the semi-ellipsoidal reflector 12 and during their discharge they produce a plasma bubble that expands and collapses transforming the heat into kinetic energy in the form of acoustic pressure shockwaves that reflects on the semi-ellipsoidal reflector 12 and produces the focused acoustic pressure shockwaves 10, which are directed towards the focal point 17 (F₂ of the ellipsoidal geometry) and overall to the focal volume 18 that overlaps with the targeted lung tissue 112 to be able to perform properly the respective treatment. To get the focused acoustic pressure shockwaves 10 inside the targeted lung tissue 112, the intervertebral space 115 is used to prevent any attenuation and loss of energy of the shockwaves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To cover properly all the affected lung volume/area that requires medical treatment, the translation (T) and swivel (S) motions of the applicator 116 are performed by the medical operator.

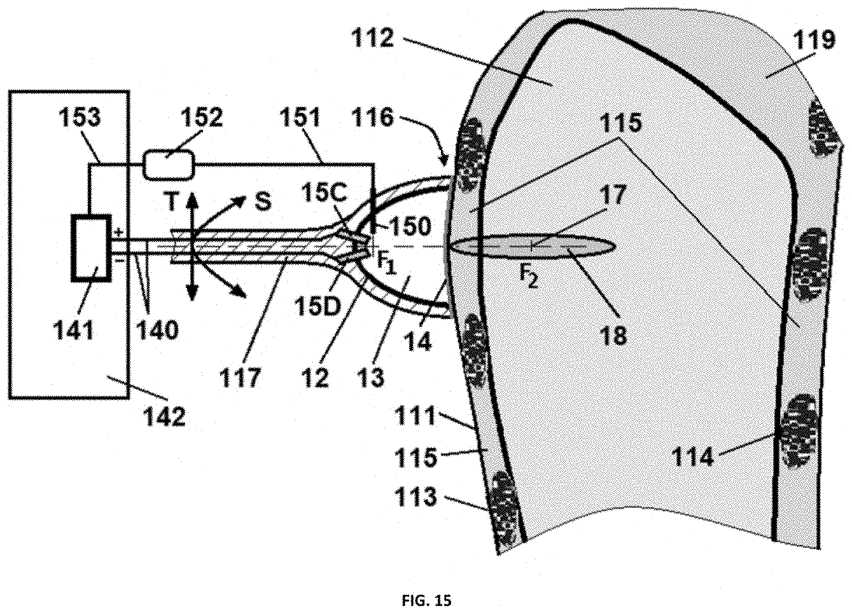

In FIG. 15 the focused acoustic pressure shockwaves 10 (shown in FIG. 14 and not shown in FIG. 15 for simplicity) are generated via one or multiple laser sources (electrohydraulic principle using one or multiple lasers sources). In this specific case the laser beams produced by first incased laser 15C and the second incased laser 15D in a fluid present inside the reflector cavity 13 generate the focused acoustic pressure shockwaves, which are then focused via semi-ellipsoidal reflector 12 towards the focal point 17 (F₂ of the ellipsoidal geometry) and overall, to the focal volume 18 that overlaps with the targeted lung tissue 112 to be able to perform properly the respective treatment. The high voltage for the first incased laser 15C and the second incased laser 15D is provided by the power source 141 (included in control/console unit 142) via high voltage cable 140. The two laser sources are positioned in such way to intersect their beams in the first focal point F₁ of the semi-ellipsoidal reflector 12 in order to produce a plasma bubble that expands and collapses transforming the heat into kinetic energy in the form of acoustic pressure shockwaves, which are then focused via semi-ellipsoidal reflector 12 towards the focal volume 18 that overlaps with the targeted lung tissue 112 to be able to perform properly the respective treatment. FIG. 15 includes a means of monitoring the system performance by measuring the reaction temperature of the plasma bubble collapse using a method of optical fiber thermometry. An optical fiber tube assembly 23 extends into the F₁ region of the semi-ellipsoidal reflector 12. The optical fiber tube assembly 150 transmits (via optical fiber 151) specific spectral frequencies created from the sonoluminescence of the plasma bubble reaction in the fluid present inside the reflector cavity 13 to the spectral analyzer 152. The loop is closed via feedback cable 153 that connects the spectral analyzer 152 with the power supply 141. Basically, the spectral analysis provided by the spectral analyzer 152 is used to adjust accordingly the power generated by the power supply 141, to ensure a proper laser discharge for the incased lasers 15C and 15D. To get the focused acoustic pressure shockwaves 10 inside the targeted lung tissue 112, the intervertebral space 115 is used to prevent any attenuation and loss of energy of the shockwaves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To cover properly all the affected lung volume/area that requires medical treatment, the translation (T) and swivel (S) motions of the applicator 116 are performed by the medical operator.

In FIGS. 14 and 15, when electrohydraulic principle is used to produce focused acoustic pressure shockwaves 10, if the semi-ellipsoidal reflector 12 is replaced with a parabolic reflector 21 that has its parabolic focal point 23 (F) in the same position as the first focal point (F₁) of the semi-ellipsoidal reflector 12, then the applicator 116 will produce in the targeted lung tissue 112 acoustic pseudo-planar pressure waves 260, similar to those from the embodiment presented in FIG. 26.

In FIG. 16 the acoustic pressure shockwaves are generated via piezo crystals/piezo ceramics 15E (piezoelectric principle using piezo crystals). In this case, the internal generation of a mechanical strain resulting from an applied electrical field to the piezo crystals/piezo ceramics 15E, which are uniformly placed on a parabolic reflector 21, generate in a fluid present inside the reflector cavity 13 the focused acoustic pressure shockwaves 40 (shown in FIG. 14 and not shown in FIG. 16 for simplicity). The focusing of the parabolic reflector 21 towards its focal point F (parabolic focal point 23) and overall, to the focal volume 18 is done with all parabolic reflector 21 surface that is covered by the piezo crystals/piezo ceramics 15E. Relatively similar effects can be accomplished when the piezo crystals/piezo ceramics 15E are used together with the semi-ellipsoidal reflector 12, but in this case since the pressure waves are originating from the surface of the semi-ellipsoidal reflector 12 and not from the focal point F₁ of the ellipsoidal geometry, the produced pressure waves fall more in the category of unfocused pressure waves and not shockwaves. This might be advantageous for lung treatments that require a lower energy, since in general the unfocused pressure waves have lower energy than the focused acoustic pressure shockwaves 10 (see comments for FIG. 5). When higher energy needs to be delivered to the targeted lung tissue 112, in those cases the use of piezo crystals/piezo ceramics 15E in combination with the parabolic reflector 21 is preferred. The electrical field for the piezo crystals/piezo ceramics 15E is provided by the power supply 141 (included in control console/unit 142) via high voltage cable 140. To get the focused shockwaves or unfocused pressure waves inside the targeted lung tissue 112, the intervertebral space 115 is used to prevent any attenuation and loss of energy of the shockwaves/pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the focal volume 18 or the pressure field produced outside the applicator/coupling membrane 14 by unfocused pressure waves needs to overlap with the targeted lung tissue 112. To cover properly all the affected lung volume/area that requires medical treatment, the translation (T) and swivel (S) motions of the applicator 116 are performed by the medical operator.

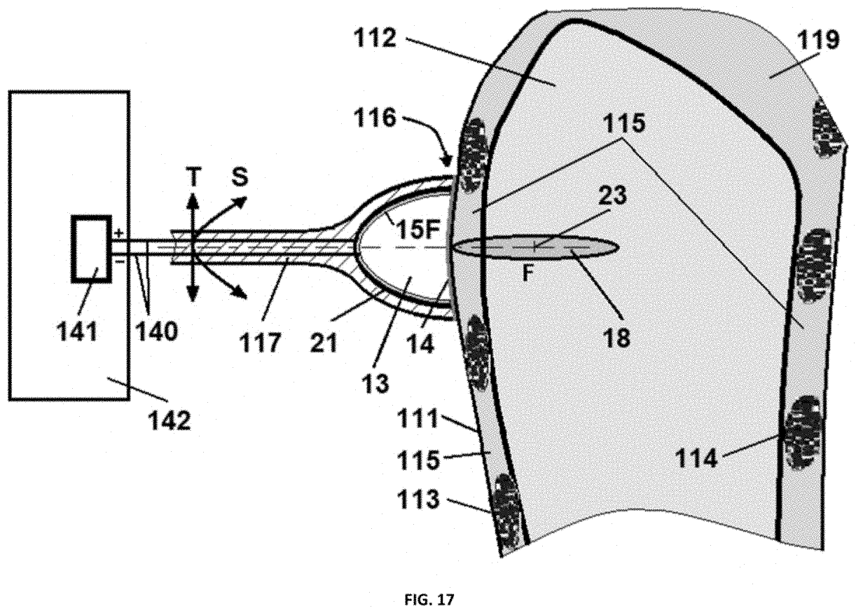

Due to the parallelepiped or cylindrical geometry of the piezo crystals/piezo ceramics 15E, they may not confirm very well to the parabolic reflector 21, which can create problems with focusing towards the parabolic focal point 23 (F), especially in situations where deep penetrations are needed, since these geometries will require a sharp vertex of the parabola with smaller radiuses that are difficult to cover with parallelepiped or cylindrical piezo crystals/piezo ceramics 15E. To overcome this issue, the piezo crystals/piezo ceramics 15E can be replaced by piezo fibers, as presented in FIG. 17. The piezo fibers can be integrated in a composite material with their longitudinal axis perpendicular to a solid surface as the parabolic reflector 21, thus forming a piezo fiber layer 15F capable of producing focused acoustic pressure shockwaves 10. The advantage of the piezo fiber layer 15F when compared to the piezo crystals/piezo ceramics 15E is that the smaller dimension and cylindrical geometry of the piezo fibers that allows them to confirm significantly better to the parabolic or ellipsoidal geometries. Furthermore, the electric contacting of the piezo fibers may be realized by a common electrically conductive layer according to the interconnection requirements. Hence, the complex interconnection of a multitude of piezo crystals/piezo ceramics 15E (as presented in FIG. 16) is no longer required. When an electrical field is provided by the power supply 141 (included in control console/unit 142) via high voltage cable 140 to the piezo fiber layer 15F, the piezo electric fibers will stretch in unison mainly in their lengthwise direction, which will create focused acoustic pressure shockwaves 10 (shown in FIG. 14 and not shown in FIG. 17 for simplicity) from the surface of the parabolic reflector 21 and towards the parabolic focal point 23 (F) and overall, to the focal volume 18. Relatively similar effects can be accomplished when the piezo fiber layer 15F is used together with a semi-ellipsoidal reflector 12, but in this case since the pressure waves are originating from the surface of the semi-ellipsoidal reflector 12 and not from the focal point F₁ of the ellipsoidal geometry, the produced pressure waves fall more in the category of unfocused waves and not shockwaves. To get the focused shockwaves or unfocused pressure waves inside the targeted lung tissue 112, the intervertebral space 115 is used to prevent any attenuation and loss of energy of the shockwaves/pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the focal volume 18 or the pressure field produced outside the applicator/coupling membrane 14 by unfocused pressure waves needs to overlap with the targeted lung tissue 112. To cover properly all the affected lung volume/area that requires medical treatment, the translation (T) and swivel (S) motions of the applicator 116 are performed by the medical operator.

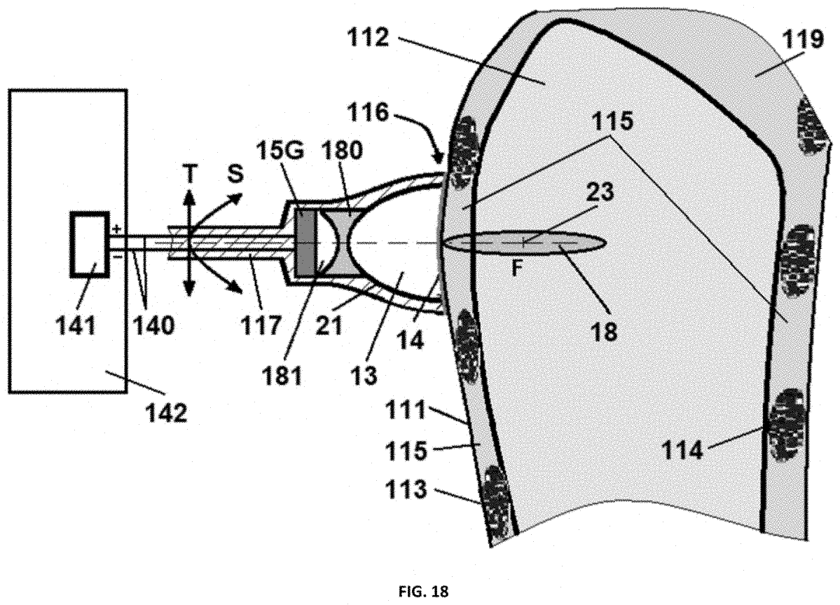

In FIG. 18 the acoustic pressure shockwaves are generated via electromagnetic flat coil and plate assembly 15G and an acoustic lens 25 (electromagnetic principle using a flat coil and an acoustic lens). In this case, an electromagnetic flat coil is placed in close proximity to a metal plate that acts as an acoustic source and thus the electromagnetic flat coil and plate assembly 15G presented in FIG. 18 is created. When the electromagnetic flat coil is excited by a short electrical pulse provided by the power supply 141 (included in control console/unit 142) via high voltage cable 140, the plate experiences a repulsive force and this is used to generate an acoustic pressure wave. Due to the fact that the metal plate is flat, the resulting acoustic pressure wave is a planar acoustic pressure wave (not shown in FIG. 18) moving in the fluid-filled cavity 181 towards the acoustic lens 180 that is focusing the planar wave and thus creating focused acoustic pressure shockwaves 10 (shown in FIG. 14 and not shown in FIG. 18 for simplicity) towards the targeted area. The focusing effect of the acoustic lens 180 is given by its shape, which as presented in FIG. 18 is a portion of a parabolic surface. This is why this is used in tandem with a parabolic reflector 21 that can help with the focusing of the produced acoustic pressure shockwaves 10 towards the parabolic focal point 23 (F) and overall, to the focal volume 18. Conversely, in another embodiment the acoustic lens 180 can be a portion of an ellipsoidal surface and in combination with a semi-ellipsoidal reflector 12 can create unfocused pressure waves that can generate a pressure field outside the applicator/coupling membrane 14 of the semi-ellipsoidal reflector 12 and inside the targeted lung tissue 112. To get the focused shockwaves or unfocused pressure waves inside the targeted lung tissue 112, the intervertebral space 115 is used to prevent any attenuation and loss of energy of the shockwaves/pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the focal volume 18 or the pressure field produced outside the applicator/coupling membrane 14 by unfocused pressure waves needs to overlap with the targeted lung tissue 112. To cover properly all the affected lung volume/area that requires medical treatment, the translation (T) and swivel (S) motions of the applicator 116 are performed by the medical operator.

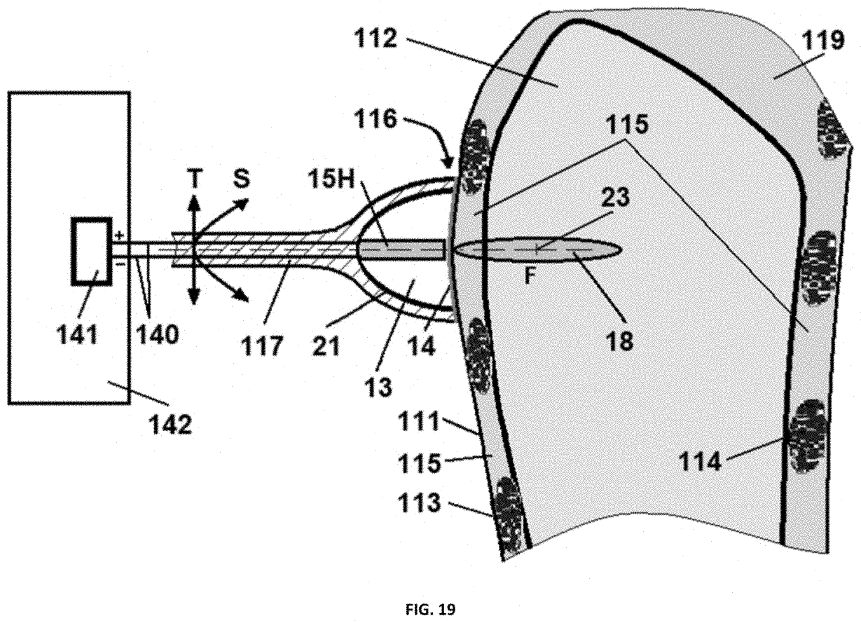

In FIG. 19 the acoustic pressure shockwaves are generated via electromagnetic cylindrical coil and tube plate assembly 1511 (electromagnetic principle using a cylindrical coil). In this case, an electromagnetic cylindrical coil is excited by a short electrical pulse provided by the power supply 141 (included in control console/unit 142) via high voltage cable 140, and the plate is in the shape of a tube (thus creating an electromagnetic cylindrical coil and tube plate assembly 1511), which will results in a cylindrical pressure wave (not shown in FIG. 19) that can be focused by a parabolic reflector 21 towards the parabolic focal point 23 (F) and overall, to the focal volume 18. When the electromagnetic cylindrical coil is excited by a short electrical pulse provided by the power supply 141 (included in control console/unit 142) via high voltage cable 140, the cylindrical coil experiences a repulsive force and this is used to generate a cylindrical acoustic pressure wave inside the fluid-filled reflector cavity 13 that is reflected on the parabolic reflector 21, thus creating focused acoustic pressure waves 10 (shown in FIG. 14 and not shown in FIG. 19 for simplicity). Conversely, in another embodiment the parabolic reflector 21 can be replaced by a semi-ellipsoidal reflector 12 to create unfocused pressure waves that generate a pressure field outside the applicator/coupling membrane 14 of the semi-ellipsoidal reflector 12 and inside the targeted lung tissue 112. To get the focused shockwaves or unfocused pressure waves inside the targeted lung tissue 112, the intervertebral space 115 is used to prevent any attenuation and loss of energy of the shockwaves/pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the focal volume 18 or the pressure field produced outside the applicator/coupling membrane 14 by unfocused pressure waves needs to overlap with the targeted lung tissue 112. To cover properly all the affected lung volume/area that requires medical treatment, the translation (T) and swivel (S) motions of the applicator 116 are performed by the medical operator.

FIG. 20 illustrates an applicator that uses hydraulic principle to generate focused shockwaves or pressure waves for the treatment of the targeted lung tissue 112 affected by a certain affliction/disease or infection. For that a parabolic reflector 21 is used to create a closed work space (reflector cavity 13), which is filled with a fluid. As symbolized by arrows 204, the pressure reflector cavity 13 is increased by pulses by fluid injection through the openings 203 disposed in a certain pattern around the cylindrical hydraulic injector 200 placed in the center and along the longitudinal axis of the parabolic reflector 21. The cylindrical hydraulic injector 200 is designed with a rigid wall that is interrupted by openings 203 uniformly distributed on a grid. Thus, using the injection pump 201 pulses of pressure are created in a liquid that is brought via hydraulic “in” tubing 202 to the cylindrical hydraulic injector 200, where is pressed under pressure through the small openings 203 into the reflector cavity 13. The fluid streams penetrating the individual openings 203 produce localized spherical pressure waves/radial pressure waves (similar to those presented in FIGS. 24 and 25) in the fluid enclosed in the reflector cavity 13. The localized spherical pressure waves/radial pressure waves combine due to the uniform distribution of the openings 203 on the cylindrical hydraulic injector 200 and thus producing an even cylindrically expanding pressure wave (similar in principle to the one mentioned for FIG. 19, and not showed in FIG. 20 for simplicity), which is focused by means of the parabolic reflector 21. To not over-pressurize the reflector space with fluid, once the focused acoustic pressure shockwave 10 (shown in FIG. 14 and not shown in FIG. 20 for simplicity) are produced, the excess fluid is evacuated back to the injection pump 201 via the hydraulic “return” tubing 206, as indicated by the return flow arrow 205. In another embodiment the parabolic reflector 21 can be replaced by a semi-ellipsoidal reflector 12 to create unfocused pressure waves that generate a pressure field outside the applicator/coupling membrane 14 of the semi-ellipsoidal reflector 12 and inside the targeted lung tissue 112. To get the focused shockwaves or unfocused pressure waves inside the targeted lung tissue 112, the intervertebral space 115 is used to prevent any attenuation and loss of energy of the shockwaves/pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the focal volume 18 or the pressure field produced outside the applicator/coupling membrane 14 by unfocused pressure waves needs to overlap with the targeted lung tissue 112. To cover properly all the affected lung volume/area that requires medical treatment, the translation (T) and swivel (S) motions of the applicator 116 are performed by the medical operator.

The embodiment presented in FIG. 21 aims at creating acoustic radial pressure waves 210, which can be produced via a small pressure radiating element called vibrating plate 217 that is made of a spring material and has the size smaller than the intervertebral space 115. Due to spherical dispersing of the acoustic radial pressure waves 210 from the vibrating plate 217, it allows the waves to be injected via intervertebral space 115 into a relatively large area of the targeted lung tissue 112, including even under the ribs. The applicator 116 comprise a reciprocating pneumatic-driven bullet 215 guided inside of a pneumatic cylinder 216 that has at its proximal end (near the pneumatic compressor 212) a magnet 219A and at the distal end the vibrating plate 217. The length of the pneumatic cylinder 216 may range between approximately 50 and 250 mm. The reciprocating pneumatic-driven bullet 215 is actuated by the pneumatic pushing pressure 214 produced by the pneumatic compressor 212 and transmitted towards the pneumatic cylinder 216 through the pneumatic “in” tubing 213. To not decelerate the reciprocating pneumatic-driven bullet 215 on its way to the vibrating plate 217, when actuated by the pneumatic pushing pressure 214, there is a pneumatic “return” tubing 219 that allows free flowing of air from in front of the reciprocating pneumatic-driven bullet 215 to the pneumatic compressor 212. The beating frequency of the pneumatic-driven bullet 215 amounts to approximately 1 to 30 Hz, preferably to approximately 2 to 20 Hz. When the reciprocating pneumatic-driven bullet 215 reaches the final pneumatic bullet position 215A, it hits with high velocity (10 to 30 m/sec, depending on the lower or higher energy needed for the lung treatment) the vibrating plate 217 and thus producing the acoustic radial pressure waves 210 in the center F of semi-spherical reflector portion 211A of the combination semi-spherical and cylindrical reflector 211 that has in its upper part the cylindrical reflector portion 211B. Due to this special construction only the direct acoustic radial pressure waves 210 emanating from the vibrating plate 217 will enter the targeted lung tissue 112, with all the other spheric waves/radial waves that are reaching the reflecting surface of the combination semi-spherical and cylindrical reflector 211 being reflected back towards the spherical center F or the longitudinal axis of the reflector. This avoids unnecessary reflected acoustic radial pressure waves 210 directed towards the patient's skin 111, which can only produce discomfort for the patient. After the reciprocating pneumatic-driven bullet 215 reaches the final pneumatic bullet position 215A, the spring material of the vibrating plate 217 springs back the reciprocating pneumatic-driven bullet 215, which allows the pneumatic “return” tubing 219 to be used to provide positive pressure on the frontal face of the reciprocating pneumatic-driven bullet 215 and thus pushing back the reciprocating pneumatic-driven bullet 215 towards the magnet 219A, where it stays in between active pneumatic cycles. The way the pneumatic pressure is directed via the pneumatic “in” tubing 213 or the pneumatic “return” tubing 219 is done by the pneumatic compressor 212 under the control of the control console/unit 142. The power supply 141 produces the energy to activate both the pneumatic compressor 212 and of the control console/unit 142 and its associated software (not specifically described into this patent from its structural and functional points of view).

As presented in the U.S. Pat. No. 6,413,230 patent, in general the applicators 116 that uses the pneumatic principle use a direct contact of the vibrating plate 217 in direct contact with the skin 111, which in order to not produce bruising limits their maximum pressure. To be able to apply higher treatment pressures, the embodiment from FIG. 21, uses the combination semi-spherical and cylindrical reflector 211 and the soft or semi-hard applicator/coupling membrane 14, which completely eliminates the direct contact of the vibrating plate 217 with the skin 111. Also, the fact that the embodiment from FIG. 21 uses a soft or semi-hard applicator/coupling membrane 14 (shaped as a flat circle for maximum lung penetration or as a cylinder to reduce penetration) against the skin 111 to send the acoustic radial pressure waves 210 towards the targeted lung tissue 112, it should be better tolerated by the patient when compared with the hard tip/coupler (similar to vibrating plate 217) of the existing pneumatic devices (see U.S. Pat. No. 6,413,230). To get the acoustic radial pressure waves 210 inside the targeted lung tissue 112, the intervertebral space 115 is used to prevent any attenuation and loss of energy of the pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the radial pressure waves 210 needs to overlap with the targeted lung tissue 112. To cover properly all the affected lung volume/area that requires medical treatment, the translation (T) and swivel (S) motions of the applicator 116 are performed by the medical operator.

As presented in FIG. 22, the effective treatment of the targeted lung tissue 112 can be accomplished by designing special full-ellipsoidal applicator 220 with small reflector's aperture “a” to match the intervertebral space 115 (opening in between frontal portion of vertebras/ribs 113 or posterior portion of the vertebras/ribs 114, depending on the direction of applying the treatment—from the front or from the back of the patient body 119). The special full-ellipsoidal applicator 220 is using deep ellipsoidal reflectors (have a larger major elliptical semiaxis “c”, as defined in FIG. 33 and showed in FIG. 22) to allow the specific construction of the special full-ellipsoidal applicator 220 and possible necessary penetration to reach deeper inside the targeted lung tissue 112. To define a deeper ellipsoidal geometry the ratio in between the major elliptical semiaxis “c” and the minor elliptical semiaxis “b” should be larger than 1.9 (c/b≥1.9). The dimension of the reflector's largest diameter Φ can be 10-100 mm, preferable 10-40 mm. When the reflector's diametral dimensions are in the range of 10-40 mm, the input high voltage discharge in F₁ (spark-gap 11 formed by the first electrode 15A and second electrode 15B) or the voltage applied for electromagnetic or piezoelectric approach is reduced to 1-14 kV or even lower, and for the larger dimensions (50-100 mm) the input voltage used to produce shockwaves or pressure waves can be in between 14-30 kV. The proposed construction special full-ellipsoidal applicator 220 is using 80-90% of the ellipsoid (increased reflective area surface), compared with classic approach (semi-ellipsoidal reflector 12 used in FIGS. 14 and 15) where only 50% of the ellipsoid surface is used to focus the pressure shockwaves. The use of 80-90% of the ellipsoidal surface is done by combining a lower shell 221 with a distinctive upper shell 222, which together form most of the internal surface of the ellipsoid, as is seen in FIG. 22. This design provides a much higher efficiency in shockwave transmission, focusing, and a larger reflector cavity 13 filled with a fluid that plays a role in producing, focusing, and transmitting the shockwaves towards the targeted lung tissue 112. The top portion of the special full-ellipsoidal applicator 220 has a conical narrow coupling membrane 223 that sits on top of a small aperture “a” of 5-10 mm that fits the intervertebral space 115 and allows only the concentration of the reflected shockwaves towards the focal point 17 and focal volume 18 without interference. Depending on the height of the conical narrow coupling membrane 223, the focal volume 18 can be placed superficial (taller membrane) or deeper (flat membrane or minimal height) inside the targeted lung tissue 112. To get the focused acoustic pressure shockwaves 10 inside the targeted lung tissue 112, the intervertebral space 115 is used to prevent any attenuation and loss of energy of the shockwaves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction/disease or infection using the special full-ellipsoidal applicator 220, the focal volume 18 produced outside the conical narrow coupling membrane 223 needs to overlap with the targeted lung tissue 112. To cover properly all the affected lung volume/area that requires medical treatment, the translation (T) and swivel (S) motions of the applicator 116 are performed by the medical operator (not specifically showed in FIG. 22, but seen in FIGS. 14-21).

In another embodiment shown in FIG. 23 the applicator 16 uses a combination semi-spherical and conical reflector 230 (combines the semi-spherical reflector portion 230A with the conical reflector portion 230B) that sends acoustic radial pressure waves 210 inside the targeted lung tissue 112 from the patient body 119 (human or animal). The combination semi-spherical and conical reflector 230 has only a central point F (the center of the semi-spherical reflector portion 230A) where the acoustic radial pressure waves 210 are generated (via the high voltage discharge between first electrode 15A and second electrode 15B inside a liquid medium from reflector cavity 13) and propagate directly through the applicator/coupling membrane 14 and intervertebral space 115 into the targeted lung tissue 112. Only the direct acoustic radial pressure shockwaves 210 are transmitted inside the targeted lung tissue 112, since the reflected waves on the bottom surface of the combination semi-spherical and conical reflector 230 will be sent back towards point F by the semi-spherical reflector portion 230A and the conical reflector portion 230B will reflect waves back towards the longitudinal axis of the combination semi-spherical and conical reflector 230. By their nature, the primary/direct acoustic radial pressure shockwaves 210 are unfocused and thus they move inside the targeted lung tissue 112 away from their point of origin F without being able to be concentrated in a certain focal region, as seen before for the acoustic pressure shockwaves 10 that are focused (schematically shown in FIG. 14). Along their way inside the targeted lung tissue 112, the acoustic radial pressure shockwaves 210 deposit their energy into the lung tissue, until all of their energy is consumed. In other words, the acoustic radial pressure shockwaves 210 have their maximum energy superficially near the skin 111 (at the entrance into the patient body 119 either human or animal) and become weaker as they travel further inside the targeted lung tissue 112. This means that is preferable to use this embodiment presented in FIG. 23 to treat the lung tissues that are more superficial and do not need deep penetration of the acoustic radial pressure shockwaves 210. In general, the acoustic radial pressure shockwaves 210 penetrations are controlled by the input energy delivered by the power supply 141 of the control console/unit 142, via the high voltage cable 140. For electrohydraulic systems/devices the input energy from control console/unit 142 is the high voltage discharge in between electrodes 15A and 15B or the voltage necessary to actuate the incased lasers 15C and 15D. For piezoelectric systems/devices the input energy from control console/unit 142 is the high voltage that excite the piezo crystals/piezo ceramics 15E or the piezo fiber layer 15F and for electromagnetic systems/devices is the current necessary to activate the electromagnetic flat coil and plate assembly 15G or electromagnetic cylindrical coil and tube plate assembly 1511. To get the acoustic radial pressure waves 210 inside the targeted lung tissue 112, the intervertebral space 115 is used to prevent any attenuation and loss of energy of the pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the acoustic radial pressure waves 210 needs to overlap with the targeted lung tissue 112. To cover properly all the affected lung volume/area that requires medical treatment, the translation (T) and swivel (S) motions of the applicator 116 are performed by the medical operator.

In FIG. 24 the acoustic pressure waves are generated via multiple high voltage discharges (electrohydraulic principle using high voltage discharges across the spark-gap 11 seen in FIG. 1) produced on the longitudinal axis of the applicator 116. As shown in this figure, there are three high voltage discharges produced in a fluid-filled volume 240 created in between the applicator/coupling membrane 14 (shaped as a cylinder) and applicator body 117. The first spark-gap discharge in F₁ is produced in between first electrode 15A and the second electrode 15B, the second spark-gap discharge in F₂ is produced in between third electrode 15A′ and the fourth electrode 15W, and the third spark-gap in F₃ is produced in between fifth electrode 15A″ and the sixth electrode 15W′. The high voltage for each pair of electrodes is provided from independent power supplies to allow a proper discharge without interference from the other pairs of electrodes. Thus, the discharge in F₁ produced in between first electrode 15A and the second electrode 15B is powered by the first power supply 141A, the discharge in F₂ produced in between third electrode 15A′ and the fourth electrode 15W is powered by the second power supply 141B, and the discharge in F₃ produced in between fifth electrode 15A″ and the sixth electrode 15W′ is powered by the third power supply 141C. The power supplies 141A, 141B, and 141C are all included in the control console/unit 142 and are connected with the applicator 116 via high voltage cable 140. In an alternative embodiment a single power supply 141 (as seen in FIGS. 14-21, and 23) can be used to power all three pairs of electrodes. For this embodiment, the first pair of electrodes 15A and 15B, the second pair of electrodes 15A′ and 15W, and the third pair of electrodes 15A″ and 15W′ can be activated concomitantly or sequentially, based on specific needs of the treatment. Furthermore, only two pairs of electrodes can be used or even only one of the pairs of electrodes can be activated, which can tailor the treatment on delivering the acoustic radial pressure waves 210 on specific locations and different tissue penetration.

There is no reflector present in this embodiment presented in FIG. 24 and this is why when the spark-gap discharges are produced, the associated plasma bubbles expand and collapse transforming the heat into kinetic energy in the form of spherical waves known as acoustic radial pressure waves 210. Due to the spherical nature of the acoustic pressure waves produced by the applicator 116 of this embodiment, the acoustic radial pressure waves 210 will propagate in all directions. The applicator 116 has only a portion of the cylindrical applicator/coupling membrane 14 in contact with the chest or patient body 119, and this is why only a portion of the spherical pressure waves/acoustic radial pressure waves 210 are transmitted through the skin 111 and the intervertebral space 115 into the targeted lung tissue 112. The rest of the spherical pressure waves/acoustic radial pressure waves 210 are transmitted away from the patient body 119 into the air. However, the symmetrical and rotational nature of the applicator 116, offer ease-of-use for the medical personnel, who does not have to choose a preferred/specific position for the applicator 116 during treatment. Being produced via electrohydraulic principle makes the acoustic radial pressure waves 210 more powerful when compared with radial pressure waves produced via pneumatic/ballistic means (see U.S. Pat. No. 6,413,230), which is most common method to produce medical acoustic radial pressure waves 210. Also, the fact that the embodiment from FIG. 24 uses a soft or semi-hard applicator/coupling membrane 14 (shaped as a cylinder) against the skin 111 to send the acoustic radial pressure waves 210 towards the targeted lung tissue 112, it should be better tolerated by the patient when compared with the hard tip/coupler of the existing pneumatic devices (see U.S. Pat. No. 6,413,230). To get the acoustic radial pressure waves 210 inside the targeted lung tissue 112, the intervertebral space 115 is used to prevent any attenuation and loss of energy of the pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the acoustic radial pressure waves 210 needs to overlap with the targeted lung tissue 112. To cover properly all the affected lung volume/area that requires medical treatment, the translation (T), swivel (S) and longitudinal (L) motions of the applicator 116 are performed by the medical operator.

In FIG. 25 the acoustic pressure waves are generated via multiple laser sources (electrohydraulic principle using multiple lasers sources) produced on the longitudinal axis of the applicator 116. There are three pairs of incased lasers that are producing laser beams and plasma bubbles in a fluid-filled volume 240 created in between the applicator/coupling membrane 14 (shaped as a cylinder) and applicator body 117. The first incased laser 15C and the second incased laser 15D represent the first pair of encased lasers that produces laser beams in F₁, the third incased laser 15C′ and the fourth incased laser 15D′ represent the second pair of encased lasers that produces laser beams in F₂, and the fifth incased laser 15C″ and sixth incased laser 15D″ represent the third pair of encased lasers that produces laser beams in F₃. In one embodiment, the high voltage for each pair of encased lasers is provided from independent power supplies. Thus, the first incased laser 15C and the second incased laser 15D are powered by the first power supply 141A, the third incased laser 15C′ and the fourth incased laser 15D′ are powered by the second power supply 141B, and the fifth incased laser 15C″ and sixth incased laser 15D″ are powered by the third power supply 141C. In another embodiment all the lasers are powered by only one power source/supply 141, as seen in FIGS. 14-21, and 23, that uses laser splitters (not shown) to split energy in between different encased lasers. Regardless of design, the power source/supply or sources are included in the control console/unit 142 and are connected with the applicator 116 via high voltage cable 140. For this embodiment, the first pair of encased lasers 15C and 15D, the second pair of encased lasers 15C′ and 15D′, and the third pair of encased lasers 15C″ and 15D″ can be activated concomitantly or sequentially. Furthermore, only two pairs of encased lasers can be used or even only one of the pairs of encased lasers can be activated, which can tailor the treatment on delivering the acoustic radial pressure waves 210 on specific locations and different tissue penetration.

To control the good functionality of the lasers there are means of monitoring the system performance by measuring the reaction temperature of the plasma bubble collapse using a method of optical fiber thermometry, which are not specifically shown in FIG. 25, but were shown in detail in FIG. 15. Also, in this embodiment there is no reflector present in this embodiment and this is why when the spark-gap discharges are produced, the associated plasma bubbles expand and collapse transforming the heat into kinetic energy in the form of spherical waves known as acoustic radial pressure waves 210. Due to the spherical nature of the acoustic pressure waves produced by the applicator 116 of this embodiment, the acoustic radial pressure waves 210 will propagate in all directions. The applicator 116 has only a portion of the cylindrical applicator/coupling membrane 14 in contact with the chest or patient body 119, and this is why only a portion of the spherical pressure waves/acoustic radial pressure waves 210 are transmitted through the skin 111 and the intervertebral space 115 into the targeted lung tissue 112. The rest of the spherical pressure waves/acoustic radial pressure waves 210 are transmitted away from the patient body 119 into the air. However, the symmetrical and rotational nature of the applicator 116, offer ease-of-use for the medical personnel, who does not have to choose a preferred/specific position for the applicator 116 during treatment. To get the acoustic radial pressure waves 210 inside the targeted lung tissue 112, the intervertebral space 115 is used to prevent any attenuation and loss of energy of the pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the acoustic radial pressure waves 210 needs to overlap with the targeted lung tissue 112. To cover properly all the affected lung volume/area that requires medical treatment, the translation (T), swivel (S) and longitudinal (L) motions of the applicator 116 are performed by the medical operator.

In the embodiment shown in FIG. 26 the applicator 116 uses a parabolic reflector 21 that sends acoustic pseudo-planar pressure waves 260 outside the applicator/coupling membrane 14 and inside the targeted lung tissue 112 from the patient body 119 (human or animal). The parabolic reflector 21 has only a central point/focus point F (parabolic focal point 23) where radial acoustic pressure waves 210 are generated (via the high voltage discharge between first electrode 15A and second electrode 15B in the liquid present inside the reflector cavity 13). The acoustic radial pressure waves 210 propagate and reflect on the parabolic reflector 21 at different time points, which creates secondary pressure wave fronts (not shown on FIG. 26 to keep clarity), especially at the edge/aperture of the parabolic reflector 21. The combination of direct acoustic radial pressure waves 210 with the secondary pressure wave fronts creates acoustic pseudo-planar pressure waves 260 outside the applicator/coupling membrane 14. In this case presented in FIG. 26, the parabolic focal point 23 is present inside the parabolic reflector 21 of the applicator 116 and this is why acoustic pseudo-planar pressure waves 260 are produced outside the applicator/coupling membrane 14 and inside the targeted lung tissue 112. This is different from the embodiments presented in FIGS. 16-20, where the parabolic focal point 23 is outside the parabolic reflector 21 of the applicator 116 and this is why focused acoustic pressure shockwaves 10 are produced outside the applicator/coupling membrane 14 and inside the targeted lung tissue 112.

By their nature, the acoustic pseudo-planar pressure waves 260 (exiting through the aperture of the parabolic reflector 21 and the applicator/coupling membrane 14) are unfocused and thus they move inside the targeted lung tissue 112 away from their point of origin F (parabolic focal point 23) without being able to be concentrated in a certain focal region, as seen before for the acoustic pressure shockwaves 10 that are focused (shown in FIGS. 16-20). Along their way inside the targeted lung tissue 112, the acoustic pseudo-planar pressure waves 260 deposit their energy into the infected/affected lung tissue, until all of their energy is consumed. In other words, the acoustic pseudo-planar pressure waves 260 have their maximum energy superficially near the skin 111, at the entrance into the patient body 119 (human or animal), and become weaker as they travel further inside the targeted lung tissue 112. This means that is preferable to use this embodiment presented in FIG. 26 to treat the targeted lung tissue 112 that is more superficial or immediate under the ribs. The penetrations of the acoustic pseudo-planar acoustic pressure waves 260 are controlled by the input energy, delivered via the cable 140 from the power supply 141 that is controlled by the control console/unit 142, in the form of high voltage setting for electrohydraulic and piezoelectric devices and electrical current setting for electromagnetic devices. To get the acoustic pseudo-planar acoustic pressure waves 260 inside the targeted lung tissue 112, the intervertebral space 115 is used to prevent any attenuation and loss of energy of the pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the acoustic pseudo-planar acoustic pressure waves 260 needs to overlap with the targeted lung tissue 112. To cover properly all the affected lung volume/area that requires medical treatment, the translation (T) and swivel (S) motions of the applicator 116 are performed by the medical operator.

In FIG. 27 an embodiment is presented that is capable to easily generate acoustic planar pressure waves 270 by using flat piezo crystals/piezo ceramics 15E. These piezoelectric devices or medical systems can be used to generate acoustic planar pressure waves 270, and direct them inside the patient body 119 (human/animal) to treat the targeted lung tissue 112. In such embodiment, the applicator 116 is preferably moved in any direction around/along the targeted lung tissue 112, via translation (T), swivel (S) and longitudinal (L) motions. It is preferably that the movement of the applicator 116 be done in such way “to paint” all the targeted area affected by disease or infection inside the lung. In this case the internal generation of a mechanical strain resulting from an applied electrical field to the piezo crystals/piezo ceramics 15E uniformly placed on the supporting column 271 (can be cylindrical, square, hexagonal, octagonal or decagonal, etc.) are producing acoustic planar pressure waves 270 inside the fluid-filled applicator/coupling membrane 14. The electrical field for the piezo crystals/piezo ceramics is provided via high voltage cable 140 by the power supply 141, which is included in control console/unit 142. To get the acoustic planar pressure waves 270 inside the targeted lung tissue 112, the intervertebral space 115 is used to prevent any attenuation and loss of energy of the pressure waves. For this embodiment, individual or multiple piezo crystals/piezo ceramics 15E can be activated concomitantly or sequentially, which can tailor the delivery of the acoustic planar pressure waves 270 on specific locations and different tissue penetration, based on explicit needs of the treatment.

The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the acoustic planar pressure waves 270 needs to overlap with the targeted lung tissue 112.

For FIGS. 11-15 the acoustic pressure shockwaves produced are reflected/focused by the semi-ellipsoidal reflector 12 or inclined semi-ellipsoidal reflector 110 towards the second focal point F₂ (focal point 17) of the ellipsoid via the contact of the applicator 116 with patient's skin 111. Only a half of an ellipsoid is used (semi-ellipsoidal reflector 12 or the inclined semi-ellipsoidal reflector 110) for focusing, to allow the transmission of the acoustic pressure shockwaves 10 (shown in FIG. 14) inside the targeted lung tissue 112 and patient body 119 (human/animal), where the second focal point F₂ (focal point 17) and the focal volume 18 are found. In this way the applicator 116 can be placed against the skin 111 of the patient body 119 (human/animal), via applicator/coupling membrane 14 and ultrasound gel (not specifically showed in any of the figures from this patent). In FIG. 22 a special full-ellipsoidal applicator 220 was presented, where by using the combination of a lower shell 221 and an upper shell 222, almost a full ellipsoidal reflector is created, which has the advantage of concentrating and focusing more energy (more potent acoustic pressure shockwave 10) in the targeted lung tissue 112, via the intervertebral space 115. For FIG. 18 the acoustic pressure shockwaves 10 are focused towards the targeted area by the acoustic lens 180 (it has the shape of a portion of a parabolic surface) plus an additional parabolic reflector 21 and for FIGS. 16, 17, 19, and 20 the focusing is realized by the parabolic reflector 21 itself. Due to the fact that different pressures fronts (direct or reflected) reach the second focal point F₂ (focal point 17 for ellipsoidal geometry of the semi-ellipsoidal reflector 12) or focus point F (parabolic focal point 23 for parabolic geometry of the reflector 21) with certain small-time differences, the focused acoustic pressure shockwaves 10 are in reality concentrated or focused on a three-dimensional space around second focal point F₂/focus point F, which is called focal volume 18. Inside the focal volume 18 are found the highest-pressure values for each focused acoustic pressure shockwave 10, which means that is preferable to position the targeted lung tissue 112 for the treatment in such way to intersect the focal volume 18 and if possible centered on the second focal point F₂ (focal point 17 for ellipsoidal geometries) or on the focus point F (parabolic focal point 23 for parabolic geometries). In order to be effective in the treatment of targeted lung tissue 112, the applicator 116 and its components are designed in such way to ensure that the focal volume 18 (where acoustic pressure shockwaves 10 are focused) is positioned correctly to allow its overlap with targeted lung tissue 112 volume, as presented in FIGS. 11-20, and 22. Conversely, if for FIGS. 11-15 a parabolic reflector 21 is used instead of a semi-ellipsoidal reflector 12 and for FIGS. 16-20 a semi-ellipsoidal reflector 12 is used instead of a parabolic reflector 21, unfocused pressure waves are generated, which create outside of the applicator/coupling membrane 14 of the applicator 116 a pressure field that overlaps with the targeted lung tissue 112 volume to produce the desired treatment of lung affliction/disease or infection. Based on the needs for the treatment of each specific lung a certain solution can be used to produce more or less energy in the targeted lung tissue 112, which offers a lot of flexibility for the medical personnel performing the treatment.

For FIG. 21 and FIGS. 23-27 the acoustic pressure waves are not focused but rather acoustic radial pressure waves 210 or acoustic pseudo-planar pressure waves 260 or acoustic planar pressure waves 270. Compared to the focused acoustic pressure shockwave 10 from FIGS. 11-15, the acoustic radial pressure waves 210 or acoustic pseudo-planar pressure waves 260 or acoustic planar pressure waves 270 might have limited penetration based on the fact that they start losing their energy immediately after getting into the lung tissue. This is why the embodiments presented in FIG. 21 and FIGS. 23-27 can be used mainly for superficial treatments of the targeted lung tissue 112 or for treatments that require lower energy.

In the embodiment from FIG. 28 a detailed three-dimensional representation of the segmented applicator 280 is presented. In this case, instead of using the full parabolic reflector 21, only a segment is used. This is done to better fit the output of the segmented applicator 280 through the intervertebral space 115 for transmitting the acoustic pseudo-planar pressure waves 260 to the targeted lung tissue 112. Also, using the specific rectangular aperture of the segmented applicator 280 is helping the user to intuitively match easier the intervertebral space 115. Thus, the segmented parabolic reflector 281 sits inside the segmented applicator body 282. A high voltage discharge produced in between first electrode 15A and the second electrode 15B (electrohydraulic principle using spark-gap 11 high voltage discharges) in the focal point F of the segmented parabolic reflector 281 and in a liquid, present inside the segmented reflector cavity 285, generates acoustic pseudo-planar pressure waves 260. In the focal point F, the acoustic radial pressure wave 210 are generated that propagate and reflect on the segmented parabolic reflector 281 surface at different time points, which creates secondary pressure wave fronts (not shown on FIG. 28 to keep clarity), especially at the edge/aperture of the segmented parabolic reflector 281. The combination of direct acoustic radial pressure wave 210 with the secondary pressure wave fronts creates acoustic pseudo-planar pressure waves 260 outside the segmented applicator membrane 284. The segmented applicator 280 has its segmented parabolic reflector 281 residing inside the segmented applicator body 282. The segmented applicator membrane 284 has a rectangular shape and sits at the aperture/opening of the segmented parabolic reflector 281 and thus creating a segmented reflector cavity 285, which is filled with a liquid. By their nature, the acoustic pseudo-planar pressure waves 260 (exiting through the aperture of the segmented parabolic reflector 281) are unfocused and thus they move towards the targeted lung tissue 112 away from their point of origin F without being able to be concentrated in a certain focal volume, as seen before for the acoustic pressure shockwaves 10 that are focused (see FIGS. 11-20 and 22). The acoustic pseudo-planar pressure waves 260 have their maximum energy at the entrance in the targeted lung tissue 112 and become weaker as they travel further inside the lung. The acoustic pseudo-planar pressure waves 260 energy is controlled by the input energy delivered by the power supply 141, in the form of high voltage setting for electrohydraulic devices. The segmented applicators 280 have their electrical connection to the control console/unit 142, through the segmented applicator leg 283. The electrical connection to the control console/unit 142 is in the form of cable 140.

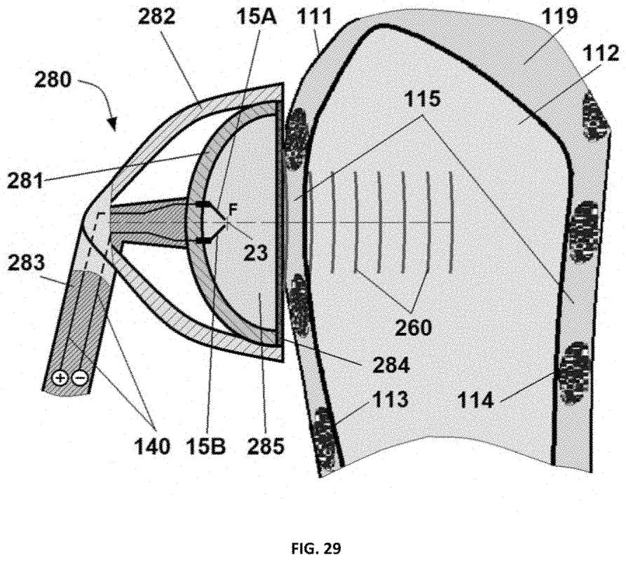

Cross-sectional views of the segmented applicators 280 are presented in FIGS. 29-32, for different principles of producing the acoustic pseudo-planar pressure waves 260.

In FIG. 29 the acoustic pseudo-planar pressure waves 260 are generated via electrohydraulic spark-gap high voltage discharges principle inside the segmented applicator 280, which has a segmented parabolic reflector 281 that resides inside the segmented applicator body 282. A segmented applicator membrane 284 sits at the aperture/opening of the segmented parabolic reflector 282 and thus creating a segmented reflector cavity 285, which is filled with a liquid. The acoustic pseudo-planar pressure waves 260 are produced via high voltage discharge produced in between first electrode 15A and the second electrode 15B at the paraboloidal focal point F in a liquid present inside the segmented reflector cavity 285. The high voltage for the first electrode 15A and the second electrode 15B is provided by the power supply 141 (not shown in FIG. 29) via electrical cable 140. The segmented applicators 280 have their electrical cable 140 to the power supply 141 via segmented applicator leg 283. The two electrodes 15A and 15B are positioned in the parabolic focal point 23 (F) of the segmented parabolic reflector 281 and during their discharge they produce a plasma bubble in the liquid from segmented reflector cavity 285 that expands and collapses transforming the heat into kinetic energy first in the form of acoustic radial pressure waves 210 (shown in FIG. 28) and outside the segmented parabolic reflector 281 in the form of acoustic pseudo-planar pressure waves 260. This represents the electrohydraulic principle to produce acoustic pseudo-planar pressure waves 260, which are transmitted through the intervertebral space 115 inside the patient's body 119 to overlap with the targeted lung tissue 112 for the respective medical treatment.

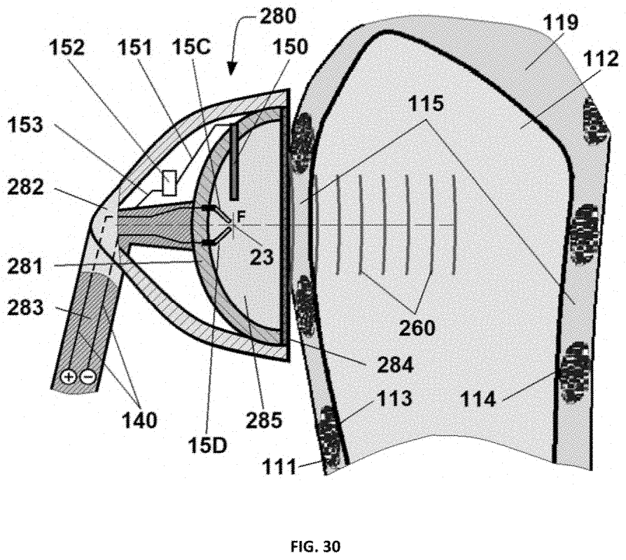

In FIG. 30 the acoustic pseudo-planar pressure waves 260 are generated via one or multiple laser sources (electrohydraulic principle using one or multiple lasers sources). The acoustic pseudo-planar pressure waves 260 are generated inside the segmented applicator 280, which has a segmented parabolic reflector 281 that resides inside the segmented applicator body 282. A segmented applicator membrane 284 sits at the aperture/opening of the segmented parabolic reflector 281 and thus creating a segmented reflector cavity 285, which is filled with a liquid. The laser beams produced by first incased laser 15C and the second incased laser 15D are positioned in such way to intersect their beams in the parabolic focal point 23 (F) of the segmented parabolic reflector 281 to produce a plasma bubble in the liquid from segmented reflector cavity 285 that expands and collapses transforming the heat into kinetic energy in the form of acoustic radial pressure waves 210 (shown in FIG. 28) and outside the segmented parabolic reflector 281 in the form of acoustic pseudo-planar pressure waves 260. The high voltage for the first incased laser 15C and the second incased laser 15D is provided by the power supply 141 (not shown in FIG. 30) via electrical cable 140. The two laser sources from FIG. 30 include means of monitoring the system performance by measuring the reaction temperature of the plasma bubble collapse using a method of optical fiber thermometry. An optical fiber tube assembly 150 extends into the parabolic focal point 23 (F) region of the segmented parabolic reflector 281. The optical fiber tube assembly 150 transmits (via optical fiber 151) specific spectral frequencies created from the sonoluminescence of the plasma reaction in the liquid present inside the segmented reflector cavity 285 to the spectral analyzer 152. The loop is closed via feedback cable 153 that connects the spectral analyzer 152 with the power supply 141 (not shown in FIG. 30) through the segmented applicator leg 283. Basically, the spectral analysis provided by the spectral analyzer 152 is used to adjust accordingly the power generated by the power supply 141 to ensure a proper laser discharge for the incased lasers 15C and 15D. The acoustic pseudo-planar pressure waves 260 produced by the laser discharge for the incased lasers 15C and 15D are transmitted through the intervertebral space 115 inside the patient's body 119 to overlap with the targeted lung tissue 112 for the respective medical treatment.

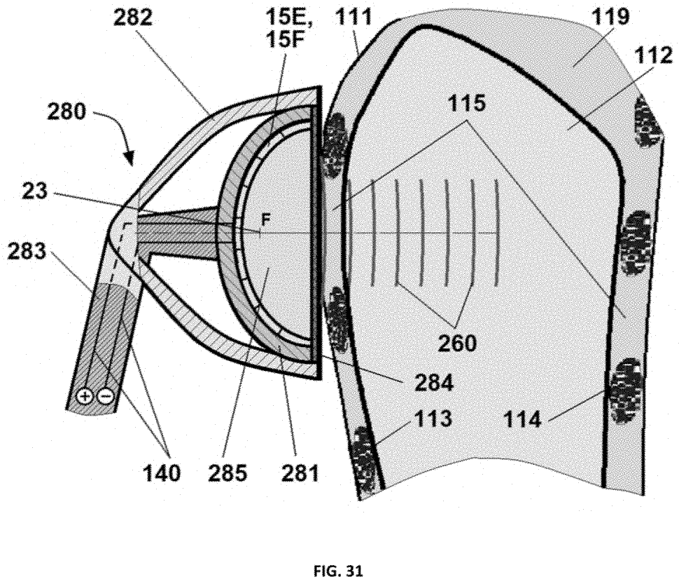

In FIG. 31 the acoustic pseudo-planar pressure waves 260 are generated via piezo crystals/piezo ceramics 15E or piezo fibers 15F (piezoelectric principle using piezo crystals/piezo ceramics or piezo fibers). In this case a mechanical strain resulting from an applied electrical field to the piezo crystals/piezo ceramics 15E or piezo fibers 15F, which are uniformly placed on the segmented parabolic reflector 281, generate in a fluid present inside the segmented reflector cavity 285 the acoustic pseudo-planar pressure waves 260. The electrical field for the piezo crystals/piezo ceramics 15E or piezo fibers 15F is provided by the power supply 141 (not shown in FIG. 31) via electrical cable 140. The segmented applicators 280 are supported and have their electrical cable 140 to the power supply 141 via segmented applicator leg 283. The segmented applicator 280 has a segmented parabolic reflector 281 that resides inside the segmented applicator body 282. A segmented applicator membrane 284 sits at the aperture/opening of the segmented parabolic reflector 281 and thus creating a segmented reflector cavity 285, which is filled with a liquid. The acoustic pseudo-planar pressure waves 260 produced by the vibration of the piezo crystals/piezo ceramics 15E or piezo fibers 15F are transmitted through the intervertebral space 115 inside the patient's body 119 to overlap with the targeted lung tissue 112 for the respective medical treatment.

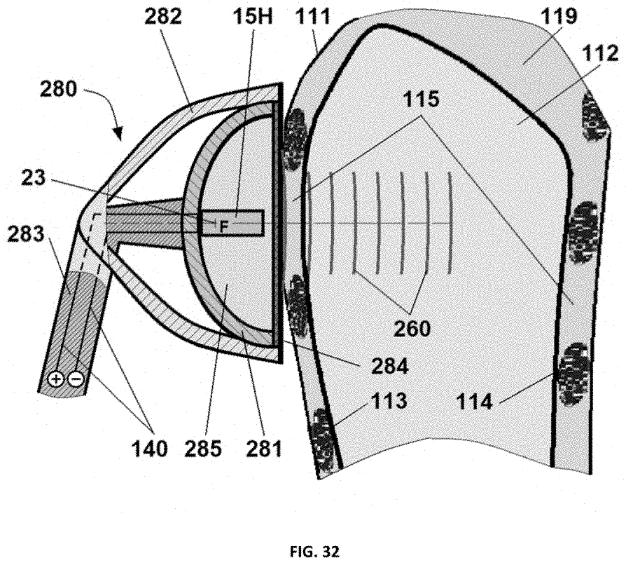

In FIG. 32 the acoustic pseudo-planar pressure waves 260 are generated via electromagnetic cylindrical coil and tube plate assembly 1511 (electromagnetic principle using a cylindrical coil). In this case, an electromagnetic cylindrical coil is excited by a short electrical pulse provided by the power supply 141 (not shown in FIG. 32) via electrical cable 140, and the plate is in the shape of a tube (thus creating an electromagnetic cylindrical coil and tube plate assembly 1511), which results in a cylindrical wave (not shown in FIG. 32) that can be focused by a segmented parabolic reflector 281 towards the targeted area via the fluid-filled segmented reflector cavity 285 of the segmented applicators 280. The segmented applicators 280 are supported and have their electrical cable 140 to the power supply 141 (not shown in FIG. 29) via segmented applicator leg 283. Similarly, to what was presented before, the segmented applicator 280 has its segmented parabolic reflector 281 residing inside the segmented applicator body 282. A segmented applicator membrane 284 sits at the aperture/opening of the segmented parabolic reflector 281 and thus creating a segmented reflector cavity 285, which is filled with a liquid. The acoustic pseudo-planar pressure waves 260 produced by the vibration of the electromagnetic cylindrical coil and tube plate assembly 1511 are transmitted through the intervertebral space 115 inside the patient's body 119 to overlap with the targeted lung tissue 112 for the respective medical treatment.

For all embodiments from FIGS. 28-32, to get the acoustic pseudo-planar acoustic pressure waves 260 inside the targeted lung tissue 112, the intervertebral space 115 is used to prevent any attenuation and loss of energy of the pressure waves. The intervertebral space 115 used for treatment is an opening in between frontal portion of vertebras/ribs 113 or posterior portion of the vertebras/ribs 114, depending on the direction of applying the treatment, either from the front or from the back of the patient body 119. To be able to perform properly the respective treatment of lung affliction, the pressure field produced outside the applicator/coupling membrane 14 by the acoustic pseudo-planar acoustic pressure waves 260 needs to overlap with the targeted lung tissue 112. To cover properly all the affected lung volume/area that requires medical treatment, the translation (T), swivel (S), and longitudinal (L) motions of the applicator 116 are performed by the medical operator (not specifically showed in FIGS. 28-32).

FIG. 33 has a geometric representation of the classic semi-ellipsoidal reflector 330, which is characterized by the major elliptical semiaxis “c”, minor elliptical semiaxis “b”, and their ratio, which dictates how shallow or deep the reflector can be. If the ration c/b is higher than 1.9 (c/b>1.9), then the reflector is considered to be deep. With a ratio of 1.1≤c/b≤1.3 the semi-ellipsoidal reflector 330 is considered shallow and for 1.3≤c/b≤1.9 is considered normal. The semi-ellipsoidal reflector 330 has also two focal points. The first focal point F₁ is where the shockwaves are generated via spark-gap 11 for the electrohydraulic principle that uses electrodes for high voltage discharge in a fluid. The shockwave focusing 16 is done by the internal surface of the classic semi-ellipsoidal reflector 330 towards the focal point 17 (second focal point F₂) and the surrounded focal volume 18, as presented in FIG. 1 too. With the construction of the classic semi-ellipsoidal reflector 330, the focal volume 18 is sectioned by the lung tissue plane in transversal direction and the focal volume 18 is projected longitudinally inside the targeted lung tissue 112, which makes it prone for deeper treatments. A different approach is to increase the treatment area in one applicator position by having the focal volume 18 sectioned by the lung tissue plane in longitudinal direction. Furthermore, in this approach the focal volume 18 is projected transversally by set it relatively parallel to the skin 111 inside the targeted lung tissue 112 along the intervertebral space 115, which makes this design more prone for superficial treatments. To accomplish that the reflector geometry is “reversed” as presented in FIGS. 34 and 35. As seen from FIG. 34, the opening/aperture of the reversed semi-ellipsoidal reflector 340 is done along the long longitudinal axis (2c) of the ellipsoid, which is different from the classic semi-ellipsoidal reflector 330 that has its aperture along the small transversal axis (2b).

For the reversed applicator 350 that contains a reversed semi-ellipsoidal reflector 340, as presented in FIGS. 34 and 35, an acoustic radial pressure wave 210 is generated/emanating from F₁ where the high voltage discharge in between electrodes 15A and 15B is produced, due to the fact that the acoustic radial pressure wave 210 that propagates below the reversed semi-ellipsoidal reflector 340 do not have any surface to bounce back towards the focal point 17 (second focal point F₂). Thus, the acoustic radial pressure waves 210 are free to propagate into the patient body 119 and inside the targeted lung tissue 112. Focused pressure shockwaves are also present, since they are produced by the acoustic radial pressure wave 210 generated in F₁ that is reflected by the upper portion of the reversed semi-ellipsoidal reflector 340 and via shockwave focusing 16 is directed towards the focal point 17 (second focal point F₂ of the ellipsoidal geometry) and the focal volume 18. In this way, the treatment area/targeted lung tissue 112 is slicing longitudinally the focal volume 18 and not transversally as it happening with the classic semi-ellipsoidal reflector 330 design, which can translate in increased efficiency of the treatment in one fixed position. The increased efficiency is given by the fact that the reversed semi-ellipsoidal reflector 340 is creating both acoustic radial pressure wave 210 and focused pressure shockwaves 10 (not showed in FIGS. 34 and 35 for clarity of the figures) acting in sequential manner on the targeted lung tissue 112 using only one reflector. The “double punch” pressure waves and shockwaves can clearly increase the efficiency of the treatment.

More specifics for the construction of the reversed applicator 350 are given in FIGS. 35 and 36. The geometry of the reversed semi-ellipsoidal reflector 340 is created by slicing the geometrical ellipsoid 351 longitudinally along the longitudinal axis of symmetry for the ellipsoid 352 and not transversally as it was done with the classic semi-ellipsoidal reflector 330 approach for a reflector geometry. In reality, to not damage the narrow coupling membrane 361 (see FIG. 36) during the high voltage discharge from the electrodes 15A and 15B and to properly expose the focal volume 18 and place it inside the targeted lung tissue 112, the actual slicing of the geometrical ellipsoid 351 is done along the contact plane with patient 353, which is not including the longitudinal axis of symmetry for the ellipsoid 352. Note that the contact with the skin 111 (not specifically showed in FIGS. 35 and 36) is done via narrow coupling membrane 361, which is covering the aperture 360 of the reversed semi-ellipsoidal reflector 340. As seen in FIG. 36, the aperture 360 and the dimension of the narrow coupling membrane 361 should match the distance in between ribs, showed as intervertebral space 115 For embodiments of the invention, to produce an efficient treatment of the targeted lung tissue 112 using the reversed applicator 350.

When shockwaves or pressure waves are produced using the electrohydraulic principle (both spark-gap or laser approaches) the applicators 116 or the segmented applicators 280 can be designed to move the spark-gap or the lasers along the longitudinal axis of the reflector in between the two focal points F₁ and F₂ of an ellipsoid. Referring to FIG. 37, the desirable shockwave focusing 16 is realized with the normal discharge in spark-gap 11 that coincides with the normal first geometric focal point F₁ of the ellipsoid, which after focusing towards the focal point 17 (second focal point F₂ of the ellipsoid) is generating inside the focal volume 18 the pressure distribution in focal volume 373. Relatively to the small axis of the ellipsoid, the focal points F₁ and F₂ are found each at the distance “c” from the semi-ellipsoidal reflector 12 edge/aperture. Simulation analysis of shockwaves or pressure waves using commercially available acoustic software packages showed that a discharge in F₁′ (pseudo spark-gap 370) away from the normal spark-gap 11 (first focal point F₁ of the ellipsoid) creates a symmetric focal point F₂′ (pseudo focal point 371) relatively to the small semiaxis of the semi-ellipsoidal reflector at reh symmetric distance “d”. Since the distance “d” is smaller than the distance “c” (d<c), the pseudo focal point 371 (F₂′) is situated before F₂ (normal geometric second focal point 17 of the ellipsoidal geometry), as seen in FIG. 37. Consequently, a pseudo focal volume 372 is formed around the pseudo focal point 371 (F₂′), where the pressure distribution in pseudo focal volume 374 shows that the pressure values are in general lower when compared to the pressure values generated when the discharge takes place in the normal spark-gap 11 (first focal point F₁ of the ellipsoidal geometry). However, the pressures included in the pseudo focal volume 372 are still higher than the surrounding ones, which makes this pseudo focal volume 372 a clearly defined entity. The normal focal volume 18 is still produced even when the high voltage discharge is done in the pseudo spark-gap 370 (F₁′). Overall, there is an overlap in the upper part in between the pseudo focal volume 372 and the normal focal volume 18, which creates an extended focal volume in longitudinal direction.

Acoustic simulation shows that the amount of energy (corelated with the pressure values) seems to be lower in the pseudo focal volume 372 centered in F₂′ when compared to the energy in the focal volume 18 centered in F₂ generated by the normal discharge in spark-gap 11 (in F₁). A defocused discharge in pseudo spark-gap 370 (F₁′) reduces the amount of energy in the corresponding pseudo focal point 371 (F₂′). If a discharge of 20 kV is used in the pseudo spark-gap 370 (F₁′) the amount of energy in the focal point 17 (F₂) and the pseudo focal point 371 (F₂′) may be similar to a 16 kV discharge. Also, sometimes it is very difficult to provide a proper electrohydraulic discharge of voltages lower than 16 kV. The variation from shock to shock can be significant or misfires can occur. By using the shifted pseudo spark-gap 370 (F₁′), the discharge can be produced at 20 kV (that is consistent for each high voltage discharge), and the energy in the corresponding pseudo focal point 371 (F₂′) can be equivalent to a 16 kV discharge. This can open consistency for the treatments that require lower energies in the targeted lung tissue 112. In this way, a broader range of energy can be delivered during treatment in the targeted lung tissue 112 using a narrow high voltage discharge range for the power supply 141. In other words, with a range of 20-28 kV for the power supply 141, it can be delivered in F₂ and F₂′ energies equivalent to 5-22 kV, when shifting from F₁ to F₁′. The high voltage discharge from 18 kV to 30 kV is used in the existing electrohydraulic devices/systems. With the shifted discharge, the range of energy delivered in the targeted lung tissue 112 treatments can be open to a full range of energies (energy flux densities (EFD) of 0.02 to 1.00 mJ/mm²) corresponding to a high voltage input from few kilovolts up to 30 kV, using one of the existing/commercially available high voltage power supply 141 incorporated in the existing commercially available control console/unit 142.

The change in focal volume longitudinal dimension (by combining the focal volume 18 with the pseudo focal volume 372) and the enlargement of the bandwidth of energies that can be delivered using a single high voltage source and one reflector geometry with shifting/movable spark-gap will be very beneficial for using focused acoustic pressure shockwaves 10 or specifically modulated pressure waves (radial 210, pseudo-planar 260, planar 270, or unfocused waves from FIG. 5) for the treatment of the targeted lung tissue 112. The longer overall focal volume (combination of the focal volume 18 with the pseudo focal volume 372) will allow a deeper penetration towards the central part of the lung, for both situations when intervertebral space 115 is used or if the treatment is performed through the ribs with associated attenuation, due to the reflection on the rib bone (mentioned before for FIG. 5). To cover properly all the affected lung volume/area, either from the front or from the back of the patient body 119, that requires the translation (T), swivel (S), and longitudinal (L) motions of the applicator 116 to be performed by the medical operator (not specifically showed in FIG. 37).

The above-mentioned approach can also be applied to the reversed semi-ellipsoidal reflector 340, as presented in FIG. 38. In general, as presented before in FIG. 35 the increased efficiency is given by the fact that the reversed semi-ellipsoidal reflector 340 is creating both acoustic radial pressure wave 210 and focused pressure shockwaves 10 (not showed in FIGS. 34 and 35 for clarity of the figures) acting in sequential manner on the targeted lung tissue 112 using only one reflector. The “double punch” pressure waves and shockwaves can clearly increase the efficiency of the treatment. With the embodiment presented in FIG. 38, another improvement in efficiency is given by the addition of the pseudo focal volume 372 that creates a longer overall focal volume due to the partial overlap with the focal volume 18. The longer overall focal volume of the reversed semi-ellipsoidal reflector 340 is sectioned longitudinally and not transversally by the targeted lung tissue 112, which translates in high pressures and energies present in the tissue at a certain level of penetration and on a larger area. The area treated by one shockwave produced by the reversed applicator 350 in one fixed position is much larger, due to the fact that the targeted lung tissue 112 is intersecting the overall focal volume longitudinally (FIG. 38) instead of transversely (FIG. 37). Adding the acoustic radial pressure wave 210 emanating from normal spark-gap 11 (F₁) or from the pseudo spark-gap 370 (F₃), this embodiment puts more energy through the intervertebral space 115 into a larger area of the targeted lung tissue 112 in one fixed positioned. Specifically, in this embodiment the electrodes 15A and 15B from spark-gap 11 (F₁) are shifted to selectively generate shockwaves at pseudo spark-gap 370 (F₃), which produces shockwave focusing from F₃ 381 and creates the overlap of the normal focal volume 18 and the pseudo focal volume 372 that intersect the body contact plane 353, which is different from the longitudinal axis of symmetry for the ellipsoid 352 of the geometrical ellipsoid 351. The variable position of the electrodes from the 15A and 15B position (F₁) to the 15A′ and 15B′ position (F₃), creates for the user a continuum of energy changes/adjustments in between a minimum and a maximum value that can be adapted/used for each specific treatment requirements for the targeted lung tissue 112. In another embodiment, the reversed applicator 350 is using only the electrodes 15A′ and 15B′ from pseudo spark-gap 370 (F₃), which will generate shockwave focusing from F₃ 382 only, which offers only one range of energies and combination of focal volumes 18 and 372. Yet in another embodiment, both pairs of electrodes (15A and 15B form the spark-gap 11 from F₁ and the 15A′ and 15B′ from F₃) are used inside the reversed applicator 350. The advantage of this embodiment is that the user can switch in between normal focusing and defocusing by just activating different pairs of electrodes, which can come handy for the health care professional. To still be able to create the continuum of energies, one of the electrode pairs can still be made shifting-possible. Fixed position for a pair or two pairs of electrodes has the advantage of eliminating movable parts, which can sometimes reduce the reliability of the reversed applicator 350 or applicator 116. Any of these embodiments for the pair of electrodes firing in F₃, or shifting pair of electrodes in between F₁ and F₃, or two pairs of electrodes positioned one in F₁ and one in F₃ and possible shifting for one of them, creates a multitude of energy options/regimens that can be applied with the reversed applicator 350 using the intervertebral space 115 or over the ribs when performing a medical treatment for the targeted lung tissue 112. This improved efficiency translates in reduced number of movements per treatment session that are necessary to cover a large treatment area. Still, to cover properly all the affected lung volume/area, either from the front or from the back of the patient body 119, that requires the translation (T), swivel (S), and longitudinal (L) motions of the reversed applicator 350 to be performed by the medical operator (not specifically showed in FIG. 38).

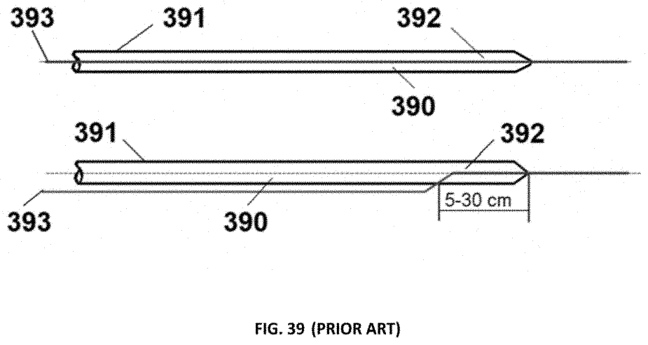

Using the natural conduits provided by the tracheal trunk and lung airways, an intracorporeal approach is possible, by using the intracorporeal catheters 390, from FIG. 39. The over-the-wire solution has the guidewire 393 going through the whole length of the intracorporeal catheter 390 (from the proximal end 391 to the distal end 392 of the catheter), which helps with a good guidance of the intracorporeal catheter 390 inside the human or animal body cavities and natural conduits/lumens. The drawback is that the relative long length (between 50 to 100 cm) of the intracorporeal catheter 390 can make its guidewire 393 exchange (retrieval from the guidewire 393 and replace with another intracorporeal catheter 390, if needed) cumbersome. Also, the guidewire 393 length must be twice the length of the intracorporeal catheter 340, which make the guidewire 393 more difficult to manipulate and more expensive too.

For rapid-exchange solution the guidewire 393 goes inside the intracorporeal catheter 390 only for 3-7.5 cm of the distal end 392 of the intracorporeal catheter 390 and for the rest of the length runs along the intracorporeal catheter 390 inside the tracheal trunk and lung airways. This reduces the length of the guidewire 393 that should be a little bit longer than the intracorporeal catheter 340 length and thus the exchange of intracorporeal catheter 390 is done much faster. This rapid-exchange solution complicates the distal end 392 design for the intracorporeal catheter 390 and not all the time is able to provide a good centering of the intracorporeal catheter 390 inside the tracheal trunk and lung airways.

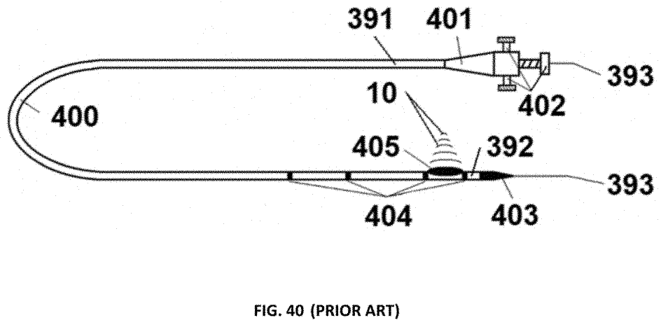

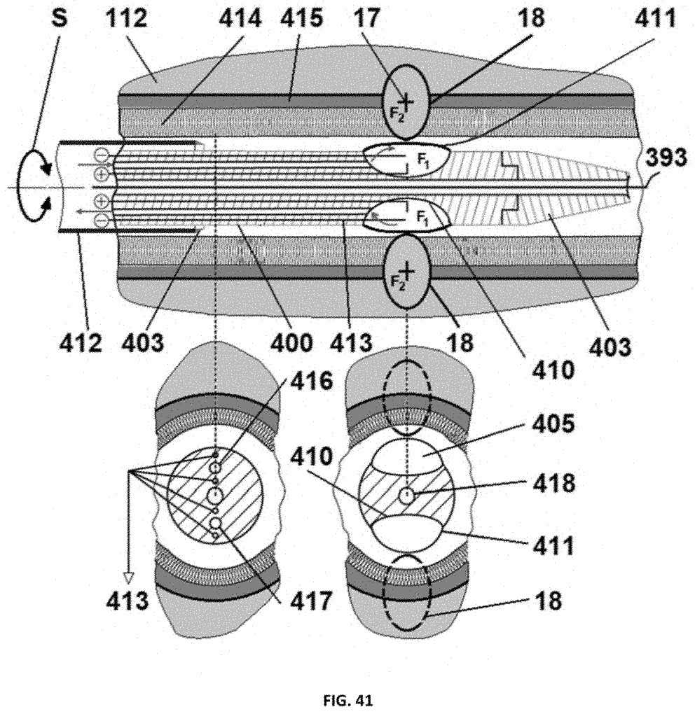

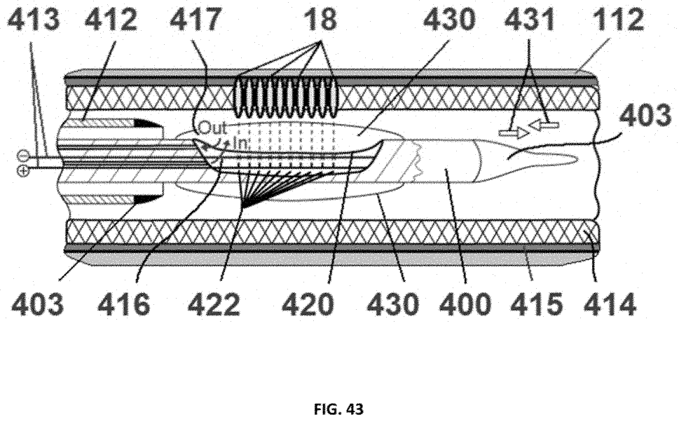

The use of intracorporeal approach to treat lung tissue, will use the natural tracheal trunk and lung airways to advance in the desired area of the lung, where the treatment of the targeted lung tissue 112 is needed. The intracorporeal approach is using special designed intracorporeal shockwave/pressure wave catheters 400, as presented in FIG. 40. The intracorporeal approach can allow the lateral exposure of the intracorporeal ellipsoidal reflector(s) 410 to access the lung conduit 415 wall the targeted lung tissue 112 from behind the lung conduit 415 and to detach mucus 414 and bacterial biofilms formed in the lung conduit 415 or in the targeted lung tissue 112, as presented in FIGS. 41 and 43. For performing the treatment correct, the intracorporeal shockwave applicators 405 of the intracorporeal shockwave/pressure wave catheters 400 must be in close contact with the lung conduit 415 and/or mucus 414, which allow the proper transmission of the shockwaves or pressure waves inside the targeted lung tissue 112.

In FIG. 40 is presented an intracorporeal shockwave/pressure wave catheters 400 functioning on the electrohydraulic principle. The intracorporeal shockwave/pressure wave catheters 400 has at the proximal end 391, access ports 402 to allow the introduction of the guidewire 393 (for “over-the-wire” construction) and for introduction/extraction of fluid from the intracorporeal shockwave applicator 405, fluid that resides inside the intracorporeal reflector cavity 419 of the intracorporeal ellipsoidal reflector 410 (see FIG. 41). The strain relief 401 is used to allow the transition from the access ports 402 to the body of the intracorporeal shockwave/pressure wave catheters 400 without kinking. The distal end 392 of the intracorporeal shockwave/pressure wave catheters 400 has the intracorporeal shockwave applicator 405, where the shockwaves or pressure waves are produced for dislodging mucus 414 or treat the targeted lung tissue 112. Also, for visualization and correct positioning in the treatment area, the distal end 392 of the intracorporeal shockwave/pressure wave catheter 400 has radio-opaque markers 404 and a radio-opaque tip 403.

The shape of the intracorporeal ellipsoidal reflector 410 of the intracorporeal shockwave applicator 405 must be shallow due to dimensional constraints and can be in the form of an ellipsoid, sphere, paraboloid, or planar. Any combination of two or more shapes can also be used. The aperture of the intracorporeal ellipsoidal reflector 410 can be circular or elongated and its dimensions (diametric) should be in the order of 2.5-10 mm, preferable 2.5-5 mm (miniature reflector) to accommodate the lung conduit 415 dimensions. The position of the intracorporeal ellipsoidal reflector 410 should be in proximity of the catheter distal end 392 (see FIG. 40).

The intracorporeal shockwave/pressure wave catheters 400 needs to be stirrable (can be rotated around its central axis of symmetry). This can be achieved by having over-the-wire construction or a rapid exchange solution for the guidewire 393, as presented in FIG. 39. The methods used to produce shockwaves or pressure waves for the intracorporeal shockwave/pressure wave catheters 400 can be electrohydraulic, electromagnetic, piezoelectric, laser discharge, explosive, hydraulic, mechanical, etc. Focused pressure shockwaves and unfocused or radial or planar or pseudo-planar pressure waves can be used. For electromagnetic principle used to produce pressure shockwaves an activator is needed, which can increase the size of the intracorporeal shockwave/pressure wave catheters 400. If piezoelectric principle is used to generate shockwaves or pressure waves, the bulkiness is reduced and thus the intracorporeal shockwave/pressure wave catheters 400 are smaller, which allows them to penetrate deeper into natural tracheal trunk and lung airways. The electrohydraulic activation principle of the shockwaves or pressure waves can be used for any of the intracorporeal application mentioned above due to their smallest diametric dimension when compared with the electromagnetic or piezoelectric principle used to generate shockwaves or pressure waves using the intracorporeal shockwave/pressure wave catheters 400.

The energy settings should be in the low energy scale (flux densities of less than 0.100 mJ/mm²), due to the dimensions of the intracorporeal ellipsoidal reflector(s) 410. The range of actuating voltages should be in between few millivolts to hundreds of volts and better in the order of volts. Also, using frequencies for shockwaves/pressure waves higher than 2 Hz and more than 1500 shockwaves or pressure waves cycles per each treatment session must be delivered to achieve the appropriate results

The intracorporeal shockwave/pressure wave catheters 400 can be deployed inside the natural tracheal trunk and lung airways and used independently or in conjunction with drug boluses (mixture of medications) to treat the affliction/disease or infection of the targeted lung tissue 112. The lung treatment can be done with the intracorporeal shockwave/pressure wave catheters 400 as an independent treatment or in conjunctions with drugs and/or extracorporeal focused acoustic pressure shockwaves 10 or specifically modulated pressure waves (radial 210, pseudo-planar 260, planar 270, or unfocused waves from FIG. 5).

When the electrohydraulic principle (as presented in FIGS. 41 and 43) is used for the intracorporeal shockwave/pressure wave catheters 400, the electrical discharge can be produced directly in any fluid present in between the intracorporeal ellipsoidal reflector 410 and intracorporeal reflector membrane 411 in the space known as intracorporeal reflector cavity 419. To have consistency of the high voltage discharge, the discharge in water or saline solution is preferred. This is why the intracorporeal shockwave/pressure wave catheters 400 has to be pre-filled or can be filled with water or saline solution at “the point of care” using the two water or saline lumens (“IN” lumen 416 and “OUT” lumen 417), as can be seen in FIGS. 41 and 43. The filling at “the point of care” is preferred, to reduce the size of the intracorporeal shockwave/pressure wave catheters 400 during advancement through natural tracheal trunk and lung airways.

For the embodiment presented in FIG. 41 the intracorporeal shockwave/pressure wave catheters 400 has multiple intracorporeal shockwave applicators 405 and associated intracorporeal ellipsoidal reflectors 410 and intracorporeal reflector membranes 411. The two associated intracorporeal ellipsoidal reflectors 410 are in communication to allow the water or saline solution to move “in” and “out” for both reflectors in the same time. The electrical discharge in the water or saline solution is done in between distal ends of the electric wires/electrodes 413 from the inside of the intracorporeal ellipsoidal reflectors 410, electric wires/electrodes 413 precisely positioned in the first focal point F₁ of the intracorporeal ellipsoidal reflectors 410. The focused acoustic pressure shockwaves 10 (not specifically shown in FIGS. 41 and 43) are sent towards the focal points 17 and associated focal volumes 18.

Multiple reflectors can increase efficiency due to the fact that the treatment is given simultaneously in multiple points of the targeted lung tissue 112, where the focal volumes 18 intersect and overlap with the mucus 414, lung conduit 415, and/or targeted lung tissue 112. The two independent intracorporeal ellipsoidal reflectors 410 showed in FIG. 41 are disposed at 180° (opposite). If the real estate permits the intracorporeal ellipsoidal reflectors 410 can be at 120° angle separation and even better at 90° angle separation. The number of intracorporeal ellipsoidal reflectors 410 can be one, two, three or more since the multiple circumferential reflectors can be duplicated or triplicated also in the longitudinal direction of the intracorporeal shockwave/pressure wave catheters 400, if the real estate permits.

The intracorporeal shockwave/pressure wave catheters 400 can be introduced by itself inside the natural tracheal trunk and lung airways under fluoroscopic guidance. However, the use of a guidewire 393 is indicated, especially to navigate more torturous lung airways. The guidewire 393 uses the dedicated guidewire lumen 418 of the intracorporeal shockwave/pressure wave catheters 400 to help with its sliding inside more torturous lung airways. Another solution is to use also guide catheters 412, which besides guiding action can be also used to suck the dislodged mucus 414 from inside the lung conduits 415. They also can be sued to introduce boluses of medication that can be activated and/or pushed inside the targeted lung tissue 112 by the shockwaves or pressure waves produce by the intracorporeal shockwave/pressure wave catheters 400. To proper navigate the natural tracheal trunk and lung airways under fluoroscopic guidance, the guide catheters 412 and the intracorporeal shockwave/pressure wave catheters 400 have radio-opaque tip 403 at their distal end or sometimes additional radio-opaque markers 404, at specific locations on their length. The guidewires 393 are visible under fluoroscopy, since they have metal components incorporated in them. To easily navigate the torturous lung airways, the intracorporeal shockwave/pressure wave catheters 400 and guide catheters 412 must be stirrable, by using the swivel (S), transversal (T), and longitudinal (L) motions.

Furthermore, in another embodiment the intracorporeal shockwave/pressure wave catheters 400 can use a reflective geometry as seen in FIG. 42. The pipe reflector 420 is made of a hypotube (thin metal tube) shaped in the form of a pipe with elliptical cross-section 423 or a parabolic cross-section 424. The elliptical cross-section 423 for the pipe reflector 420 can focus away pressure shockwaves generated by the discharge points 422 (F₁, F₂, F₃, F₄, and F₅) towards the focal volumes 18 that intersect and overlap with the mucus 414, lung conduit 415, and/or targeted lung tissue 112, as shown in FIG. 43. When the parabolic cross-section 424 is used for the pipe reflector 420, the discharge points 422 (F₁, F₂, F₃, F₄, and F₅) represent the parabolic focal points 23 (seen before in FIGS. 26 and 28-32) and pseudo-planar pressure waves will be generated outside the pipe reflector 420 inside the mucus 414, lung conduit 415, and/or targeted lung tissue 112. The discharge points 422 of the pipe reflector 420 can be all activated simultaneously or subsequently, and in other cases, only selective discharge points or individual points can be activated to match the specific requirements of the treatment and location of the mucus 414 and/or targeted lung tissue 112. Due to the sophistication necessary for the selective activation of the discharge points 422, the electric power and the activation commands are provided by the controller 421, as seen in FIG. 42.

For the embodiment from FIG. 43, the pipe reflector 420 is placed inside the non-occlusion balloon 430 to facilitate the presence of water or saline solution for all the discharge points 422 and also to produce a smooth and soft contact with the lung conduit 415. The non-occlusion balloon 430 is inflated and deflated with water or saline solution via the “IN” lumen 416 and “OUT” lumen 417. The material of the non-occlusion balloon 430 should be able to sustain high pressures (for example nylon or other materials used for angioplasty balloons). The water or saline solution can be filled at “the point of care” using the two lumens (“IN” lumen 416 and “OUT” lumen 417). The filling at “the point of care” is preferred, to reduce the size of the intracorporeal shockwave/pressure wave catheters 400 during advancement through natural tracheal trunk and lung airways. The normal air flow 431 from inside the lung conduit 415 does not provide a good conductive medium for the shockwaves/pressure waves developed in a fluid (water or saline solution) due to the acoustic impedance that produces the loss of energy at the fluid/air interface. Knowing that, the difference between the shaft of the intracorporeal shockwave/pressure wave catheter 400 and the outer diameter of the non-occlusion balloon 430 could be minimal (difference of 2-5 mm radial), but sufficient to assure a complete contact with the mucus 414 or the lung conduit 415, to assure a good transmission of the shockwaves (for the pipe reflector 420 with elliptical cross-section 423) or pseudo-planar pressure waves (for the pipe reflector 420 with parabolic cross-section 424). That is a valid point for both intracorporeal embodiments presents in FIGS. 41 and 43. Anyway, there should be a way to center the intracorporeal shockwave/pressure wave catheter 400 inside the lung conduit 415 to allow the treatment of the targeted lung tissue 112, which is done via inflation/deflation of the non-occlusion balloon 430. In other words, it is wanted that the focal volumes 18 or the pressure field of pseudo-planar waves to intersect the mucus 414 or the lung conduit 415 or targeted lung tissue 112 to provide a good treatment. In some situations, the non-occlusion balloon 430 can be inflated to be in contact all around with the lung conduit 415. To produce shockwaves (for the pipe reflector 420 with elliptical cross-section 423) or pseudo-planar pressure waves (for the pipe reflector 420 with parabolic cross-section 424), the electrical discharge in the water or saline solution is done in discharge points 422 (F₁, F₂, F₃, F₄, and F₅) that are formed by the electric wires/electrodes 413 inside of the pipe reflector 420.

Note that the solution presented in FIG. 43 does not have a guide wire lumen 418 as was seen in FIG. 41. The guiding of the intracorporeal shockwave/pressure wave catheter 400 into the treatment region is made by the guide catheter 412. Initially a guidewire 393 was used to allow the correct positioning and advancement of guide catheter 412 inside the lung conduit 415 by gliding over the guidewire 393. After this step, the guidewire 393 is retrieved and finally the intracorporeal shockwave/pressure wave catheter 400 is set in place by sliding it inside the guide catheter 412. Besides guiding purpose, the guide catheter 412 can be also used to suck out the dislodged mucus 414 from the lung conduit 415.

To cover the whole targeted lung tissue 112 the intracorporeal shockwave/pressure wave catheter 400 must be moved axially and rotate 360° using the swivel (S), transversal (T), and longitudinal (L) motions, as presented in FIG. 41. These motions inside the lung conduits 415 is done under fluoroscopic guidance using the radio-opaque tip 403 of the intracorporeal shockwave/pressure wave catheter 400 and the catheters' radio-opaque markers 404 (not shown in FIG. 43).

It is important to note that the same type of pipe reflectors 420 can be also used for the extracorporeal embodiments presented before. In this case the non-occlusion balloons 430 are replaced by the applicator/coupling membranes 14, which will set on top of the pipe reflectors 420. The elongated geometry of the pipe reflectors 420 and their aperture dimension are good to fit the intervertebral space 115. As was presented for the intracorporeal situation, for the extracorporeal solution using the pipe reflectors 420, the discharge points 422 of the pipe reflector 420 can be all activated simultaneously or subsequently, and in other cases, only selective discharge points or individual points can be activated to match the specific requirements of the treatment and location of the targeted lung tissue 112. Due to the sophistication necessary for the selective activation of the discharge points 422, the electric power and the activation commands are provided by the controller 421, as seen in FIG. 42.

For all intracorporeal solutions presented in FIGS. 40-43, the multiple points of origin for the shockwaves (multiple intracorporeal ellipsoidal reflector 410 or multiple discharge points 422, in one intracorporeal shockwave/pressure wave catheter 400) can be controlled via special software, which can allow the firing simultaneously of each point of origin or sequentially in a predetermined pattern by the controller 421, as presented in FIG. 42. The same assumption is valid when the pipe reflectors 420 are used for extracorporeal applicators 116 for the treatment of targeted lung tissue 112.

For all intracorporeal solutions presented in FIGS. 40-43, radio-opaque markers 404 (as showed in FIG. 40) on the catheter proximal end 391 of the intracorporeal shockwave/pressure wave catheter 400, in the form of a line or a marker point, will allow the user to know the proper alignment of the intracorporeal shockwave/pressure wave catheter 400 against the lung conduit 415.

For the embodiments from FIGS. 11-32, 35-36, 38, and 41-43 the penetration inside the patient body 119 (human/animal) and the geometry of the focal volume 18 (for FIGS. 11-20, and 22 only) are dictated by energy setting for acoustic pressure shockwaves 10 or specifically modulated pressure waves (radial 210, pseudo-planar 260, planar 270, or unfocused waves from FIG. 5), which is also known as “input energy”. The penetration inside the patient body 119 (human/animal) and the geometry of the focal volume 18 are also dictated by applicator/coupling membrane 14 geometry and by dimensional characteristics of the ellipsoidal reflector 12, which is dictated by the ratio of the large semi-axis and small semi-axis of the ellipsoid (see FIGS. 33 and 34), and by reflector's aperture defined as the dimension of the opening of the reflector (ellipsoidal reflector 12, or parabolic reflector 21, or inclined semi-ellipsoidal reflector 110, or combination semi-spherical and cylindrical reflector 211, or combination semi-spherical and conical reflector 230, or reversed applicator 350, or pipe reflector 420). A relatively deep reflector allows a deeper penetration in the targeted lung tissue 112. In general, to accomplish that, the ratio of the large semi-axis and small semi-axis (b/c) of the ellipsoid (their dimension is given by their intersection with the ellipsoid, and their semi-axis value being defined as half of their respective full dimensions as seen in FIG. 33) preferably have values in between 1.1 and 1.9. For the parabolic reflector 21 when used to produce focused acoustic pressure shockwaves 10 (presented in FIGS. 16-20) its geometry should be chosen in such way that the focus point of the parabola F is positioned deep enough to allow its overlap with the targeted lung tissue 112. That means that the focal length (defined as distance between the bottom of the reflector 21 where the parabola is most sharply curved and its focus point F of the parabola) for the parabolic reflector 21 should be at least 5 cm, depending on the position of the targeted lung tissue 112 inside the patient body 119 (human/animal).

For embodiments from FIGS. 21, and 23-32 the penetration inside the patient body 119 (human/animal) is dictated only by the energy setting for specifically modulated pressure waves (radial 210, pseudo-planar 260, planar 270, or unfocused waves from FIG. 5), also known as input energy. Note that the shape of the applicator/coupling membrane 14 and the unfocused nature of the acoustic radial pressure waves 210 or acoustic pseudo-planar pressure waves 260 or acoustic planar pressure waves 270, they allow a large diametral (equal to the reflector's aperture) in the targeted lung tissue 112.

For embodiments of the invention, the fluid present inside the reflectors or inside the applicator/coupling membrane 14 of the applicators 116 presented in FIGS. 11-32, 35-36, 38, and 41-43 is preferably a mixture of degassed water with proprietary substances or particles or catalysts that promote a better discharge and recombination of free radicals back to water form, as presented in U.S. Pat. Nos. 6,080,119 and 9,198,825. Other fluids may also be employed, which it will be appreciated by those of ordinary skill in the art, to provide suitable acoustic properties for producing and conducting focused acoustic pressure shockwaves 10 or acoustic radial pressure waves 210 or acoustic pseudo-planar pressure waves 260 or acoustic planar pressure waves 270 or unfocused acoustic pressure waves as presented in FIG. 5. Furthermore, for all embodiments presented in FIGS. 11-32, 35-36, 38, and 41-43, the acoustic properties of the fluid are preferably similar to the acoustic properties of human and animal bodies, which allow transmission of the focused acoustic pressure shockwaves 10 or acoustic radial pressure waves 210 or acoustic pseudo-planar pressure waves 260 or acoustic planar pressure waves 270 or unfocused waves from FIG. 5, seamlessly in between the applicator 116 and the patient body 119 (human/animal), via ultrasound coupling gel (not specifically shown in any of these figures).

For embodiments of the invention, in order to transmit acoustic pressure shockwaves inside the body, in between the applicator/coupling membrane 14 of the applicator 116/reversed applicator 350 and the skin 111 of the patient, an acoustic coupling gel (ultrasound gel) must be used, which is not specifically shown in any of the figures. The gel has the same acoustic properties as animal/human soft tissue or skin 111 and matches the acoustic impedance of the fluid enclosed inside the reflector cavity 13 of the applicator 116. In this way the transmission of the focused acoustic pressure shockwaves 10 or acoustic radial pressure waves 210 or acoustic pseudo-planar pressure waves 260 or acoustic planar pressure waves 270 or unfocused waves from FIG. 5 is done without any losses through the intervertebral space 115 into the targeted lung tissue 112. Caution must be taken, to not have air bubbles trapped inside the acoustic coupling gel, based on the fact that air can significantly interfere with the propagation and potency/energy of the focused acoustic pressure shockwaves 10 or acoustic radial pressure waves 210 or acoustic pseudo-planar pressure waves 260 or acoustic planar pressure waves 270 or unfocused waves from FIG. 5, due to significant acoustic mismatch.

For embodiments of the invention, the treatment is applied first from the frontal approach, when the focused acoustic pressure shockwaves 10 or acoustic radial pressure waves 210 or acoustic pseudo-planar pressure waves 260 or acoustic planar pressure waves 270 or unfocused waves from FIG. 5 are delivered through the intervertebral space 115 formed in between frontal portion of vertebra/rib 113. Similarly, to cover the entire volume of the targeted lung tissue 112, the treatment is also applied from the dorsal approach, when the focused acoustic pressure shockwaves 10 or specifically modulated pressure waves (radial 210, pseudo-planar 260, planar 270, or unfocused waves from FIG. 5) are delivered through the intervertebral space 115 formed in between posterior portion of vertebra/rib 114. The operator is manipulating the applicator 116 by keeping the hands on the applicator enclosure/housing 117 and using a swiveling motion S or a translation motion T or a longitudinal motion L, as needed and possible, to perform the complete treatment of the targeted lung tissue 112.

The quantity of energy deposited inside the targeted lung tissue 112 during one treatment session by the focused acoustic pressure shockwaves 10 or acoustic radial pressure waves 210 or acoustic pseudo-planar pressure waves 260 or acoustic planar pressure waves 270 or unfocused waves from FIG. 5 is dependent on the following elements:
Input energy delivered via high voltage cable 140 by the power supply 141 or the first power supply 141A or the second power supply 141B or the third power supply 141C, incorporated in the control console/unit 142 (applies to all embodiments and are specifically showed in FIGS. 14-21 and 23-28).
Output energy inside the targeted lung tissue 112 of each focused acoustic pressure shockwaves 10 or acoustic radial pressure waves 210 or acoustic pseudo-planar pressure waves 260 or acoustic planar pressure waves 270 or unfocused waves from FIG. 5, known as energy flux density or instantaneous intensity at a particular point inside the tissue condition 19
Frequency of repetition for acoustic pressure shockwaves or pressure waves, defined as number of acoustic pressure shockwaves/pressure waves per each second
Total amount of focused acoustic pressure shockwaves 10 or acoustic radial pressure waves 210 or acoustic pseudo-planar pressure waves 260 or acoustic planar pressure waves 270 or unfocused waves from FIG. 5 delivered in one treatment, known also as dosage

The amount of energy deposited into the treatment zone needs to be sufficient to allow the therapy of the targeted lung tissue 112. For embodiments of the invention, the voltage provided by the power supply 141 via high voltage cable 140 should be in the range of 1 to 30 kV for all embodiment presented in this patent.

In the embodiments from this patent, due to the fact that hard materials as bone of the ribs have the tendency to reflect focused acoustic pressure shockwaves 10 or acoustic radial pressure waves 210 or acoustic pseudo-planar pressure waves 260 or acoustic planar pressure waves 270 or unfocused waves from FIG. 5, there will be reflections at the bone/soft tissue. This occurs due to different acoustic properties of soft tissue and bone, and due to the energy incorporated in the reflected components of the shockwaves/pressure waves, a part of the initial energy is lost for the transmitted components that finally reach the targeted lung tissue 112. In cases where the applicators 116 are used not only over the intervertebral space 115, but also over the ribs, to overcome energy loses, the focused acoustic pressure shockwaves 10 or acoustic radial pressure waves 210 or acoustic pseudo-planar pressure waves 260 or acoustic planar pressure waves 270 or unfocused waves from FIG. 5 will need to be strong enough to allow the transmitted component of the acoustic pressure shockwaves/pressure waves at these interfaces to have sufficient output energy in the targeted lung tissue 112. Due to that, For embodiments of the invention, the energy flux density of each acoustic pressure shockwave or pressure waves is preferably to be in the range of 0.02 to 1.00 mJ/mm². However, depending on the characteristics of each device, the energy flux density is carefully chosen for each specific application in such way to not produce any damage to the targeted lung tissue 112.

For embodiments of the invention, the repetition rate or frequency is recommended to be in the range of 1 to 30 Hz (preferable 1-10 Hz for non-pneumatic devices and 2 to 20 Hz for pneumatic devices) for treating the targeted lung tissue 112 using focused acoustic pressure shockwaves 10 or acoustic radial pressure waves 210 or acoustic pseudo-planar pressure waves 260 or acoustic planar pressure waves 270 or unfocused waves from FIG. 5.

For embodiments of the invention, the total amount of focused acoustic pressure shockwaves 10 or acoustic radial pressure waves 210 or acoustic pseudo-planar pressure waves 260 or acoustic planar pressure waves 270 or unfocused waves from FIG. 5 is dependent on the type of affliction/disease or infection of the targeted lung tissue 112. In order to effectively treat targeted lung tissue 112, generally the initial/fixed total number of focused acoustic pressure shockwaves 10 or acoustic radial pressure waves 210 or acoustic pseudo-planar pressure waves 260 or acoustic planar pressure waves 270 or unfocused waves from FIG. 5 (not customized/personalized based on patient's comorbidities and style of life and also on the severity of the tissue condition, as presented in U.S. Pat. No. 10,888,715 for wound care) is in between about 500 and about 4,000, depending on the area/volume of the targeted lung tissue 112. If the large amount of focused acoustic pressure shockwaves 10 or acoustic radial pressure waves 210 or acoustic pseudo-planar pressure waves 260 or acoustic planar pressure waves 270 or unfocused waves from FIG. 5 is not feasible to be accomplished in a single session/treatment, then multiple sessions may be applied and spread over a certain period of time, such as twice a day or every day or every other day or two times per week, or one time per week, etc. In general, the initial/fixed number of treatments (not customized/personalized based on patient's comorbidities and style of life and also on the severity of the tissue condition) of four (4) to ten (10) sessions are preferable to be performed, followed by a resting period of few days to few weeks. If the targeted lung tissue 112 affliction/disease or infection is not completely eradicated with the first round of sessions, after recommended resting period, the focused acoustic pressure shockwaves 10 or acoustic radial pressure waves 210 or acoustic pseudo-planar pressure waves 260 or acoustic planar pressure waves 270 or unfocused waves from FIG. 5 can be administered again, without producing any side effects.

The focused acoustic pressure shockwaves 10 or acoustic radial pressure waves 210 or acoustic pseudo-planar pressure waves 260 or acoustic planar pressure waves 270 or unfocused waves from FIG. 5 can be transmitted in any angle possible relatively to the targeted lung tissue 112 without any heat loss along the pathway (regardless of the distance travelled to the targeted area), can penetrate any type of tissue (hard, semi-hard, soft). Also, the shockwaves or pressure waves can treat superficial or profound seated tissue conditions, using an extracorporeal/non-invasive approach as presented in embodiments from FIGS. 11-38 or using the intracorporeal approach as presented in FIGS. 39-43.

From the energy point of view, the shockwaves or specifically modulated pressure waves (planar, pseudo-planar, radial, or unfocused waves) applied through the intervertebral space 115 to the lungs preferably is capable to generate a flux density from 0.020 to 0.400 mJ/mm² per each pulse and the cumulative total energy delivered in one session in the targeted lung tissue 112 of the applicator preferably is equal or less than 1.6 Joule per each treatment session. When the shockwaves or specifically modulated pressure waves (planar, pseudo-planar, radial, or unfocused waves) are delivered through the ribs and not necessarily through the intervertebral space 115 the flux density ranges from 0.400 to 1.00 mJ/mm² per each pulse and the cumulative total energy delivered in one session in the targeted lung tissue 112 of the applicator preferably is equal or less than 4 Joule per each treatment session. Regarding the number of shockwave pulses or pressure wave pulses per treatment session should not exceed 4000 pulses and pulse frequency of 1-10 Hz for non-pneumatic devices and 2 to 20 Hz for pneumatic devices.

In conclusion, extracorporeal and possible intracorporeal focused acoustic pressure shockwaves 10 or specifically modulated pressure waves (radial 210, pseudo-planar 260, planar 270, or unfocused waves from FIG. 5) can be applied to the targeted lung tissue 112. For the extracorporeally solutions the shockwaves/pressure waves can be applied through the intervertebral space 115 and even through the ribs with associated attenuation, due to the reflection on the rib bone (mentioned before for FIG. 5). To cover properly all the affected lung volume/area, either from the front or from the back of the patient body 119, that requires the translation (T), swivel (S), and longitudinal (L) motions. The intracorporeal solution is applied via the natural tracheal trunk and lung airways, when the intracorporeal shockwave applicators 405 from the distal end 392 of the intracorporeal shockwave/pressure wave catheter 400 are put in direct contact with the mucus 414 or the lung conduit 415, to dislodge the mucus 414 and treat the lung conduit 415 or targeted lung tissue 112. The amount of energy deposited in the targeted lung tissue 112 can be varied in wide range based on input energy setting, construction of the reflectors, focus or defocused generation of the focused acoustic pressure shockwaves 10 or specifically modulated pressure waves (radial 210, pseudo-planar 260, planar 270, or unfocused waves from FIG. 5) and their way of transmission inside the patient body 119 (human/animal). The diverse approaches on treating the afflictions/diseases or infections for the lungs with the focused acoustic pressure shockwaves 10 or specifically modulated pressure waves (radial 210, pseudo-planar 260, planar 270, or unfocused waves from FIG. 5), offers viable solutions to treat dangerous viral infections as Severe Acute Respiratory Syndrome (SARS-CoV), porcine flu, Middle East Respiratory Syndrome (MERS), and Corona Virus Disease (COVID-19), to attenuate the “viral cytokine storm” produced by a viral infection, to produce tissue regeneration, to increase revascularization of lung tissue via angiogenesis, to prevent scar tissue formation inside the lungs, and to treat bronchitis, pneumonia, chronic obstructive pulmonary disease (COPD), idiopathic pulmonary fibrosis (IPF), and cystic fibrosis, to mention some of the most important ones.

Various embodiments of the invention have been described. It will, however, be evident that various modifications and changes may be made thereto, and additional embodiments may be implemented, without departing from the broader scope of the invention as set forth by the claims. This specification is to be regarded in an illustrative rather than a restrictive sense.

What is claimed is:

1. A method for treating a human or animal body comprising applying shockwaves or modulated pressure waves to targeted lung tissue, wherein said shockwaves or pressure waves produce compressive positive pressure during a compressive phase followed by a collapse of cavitation bubbles that produce micro jets during a tensile phase, and wherein if modulated pressure waves are applied said modulated pressure waves are selected from the group consisting of planar, pseudo-planar, radial, and unfocused pressure waves.

2. The method of claim 1, wherein said applying step comprises focusing shockwaves to said lung tissue.

3. The method of claim 2, said focusing of shockwaves originates from outside a human or animal body.

4. The method of claim 2, wherein said focusing of shockwaves originates from inside a human or animal body.

5. The method of claim 1, wherein the applying of said shockwaves or modulated waves originates from outside a human or animal body.

6. The method of claim 1, wherein the applying of said shockwaves or modulated pressure waves originates from inside a human or animal body.

7. The method of claim 6, wherein said shockwaves or pressure waves are unfocused.

8. The method of claim 5, wherein said shockwaves or pressure waves are unfocused.

9. The method of claim 2, wherein said shockwaves are applied at an angle between 20°-70° relative to the surface of the lung.

10. The method of claim 9, wherein said shockwaves are applied while continuously adjusting said angle relative to the surface of the lung between 20°-70°.

11. The method of claim 10, wherein said shockwave are applied from an applicator coupled to a hinge.

12. The method of claim 1, wherein said shockwaves or modulated pressure waves are applied with semi-ellipsoidal reflector.

13. The method of claim 1, wherein said shockwaves or modulated pressure waves are applied with a reversed semi-ellipsoidal reflector.

14. The method of claim 1, wherein said shockwaves or modulated pressure waves are applied with reflector including a combination of different geometries.

15. The method of claim 1, wherein said shockwaves or modulated pressure waves are applied with a parabolic.

16. The method of claim 1, wherein said shockwaves or modulated pressure waves deliver approximately 3 MPa of pressure to said lung tissue during a treatment.

17. The method of claim 1, further comprising treating said lung tissue with at least one of drug, stem cell, ultrasound and mechanical therapy in combination with said applying of shock waves or modulated pressure waves.

18. The method of claim 1, wherein the targeted lung tissue is affected by at least one of SARS-CoV, porcine flu, MERS, COVID-19, coronavirus, a viral cytokine storm produced by a viral infection, bronchitis, pneumonia, chronic obstructive pulmonary disease, idiopathic pulmonary fibrosis, and cystic fibrosis.

19. The method of claim 1, wherein the targeted lung tissue includes at least one of a viral infection and bacterial infection.

20. The method of claim 1, further comprising applying a sufficient energy from shockwaves or modulated pressure waves for the targeted lung tissue to least one of undergo tissue regeneration, undergo revascularization via angiogenesis, undergo scar reduction and have scar formation prevented.
